(19) United States
(12) Patent Application Publication
(10) Pub. No.: US 2025/0059855 A1
(43) Pub. Date: Feb. 20, 2025

(54) ZERO EMISSIONS ENGINE PROCESSOR, ELECTRIC PIPELINE AND ALGAE RELATED TECHNOLOGIES, ZEEP-EPART CONCEPT

(71) Applicant: Joe Travis Moore, Catoosa, OK (US)

(72) Inventor: Joe Travis Moore, Catoosa, OK (US)

(21) Appl. No.: 18/806,391

(22) Filed: Aug. 15, 2024

Related U.S. Application Data
(60) Provisional application No. 63/533,021, filed on Aug. 16, 2023.

(51) Int. Cl. E21B 41/00; A01H 13/00; B01D 53/14; B01D 53/18; C10K 1/00

(52) U.S. Cl. CPC E21B 41/0064; A01H 13/00; B01D 53/1406; B01D 53/1475; B01D 53/185; C10K 1/005

(57) ABSTRACT

An integrated process that combines Atmospheric Carbon Dioxide removal and sequestration using algae related technologies with the production and distribution of electrical power through conventional or Zero Emission methods and an underground Electric Pipeline along with the production, transportation and processing of oil, natural gas, renewable methane, renewable diesel, renewable gasoline, sustainable aviation fuel, salt water, fresh water, carbon dioxide and related coproducts and services. The process can eliminate all greenhouse gas emissions, while creating Emissions Free Energy, EFE, coproducts such as fresh water, ammonia fertilizers, plastics, chemicals, renewable energy resources, electricity, electrical energy storage, and an electrical driven transportation system.

### RELATED CASE

This application claims the benefit of U.S. Provisional Patent Application No. 63/533,021 filed Aug. 16, 2023 and incorporates said provisional application herein by reference in its entirety as if fully set forth at this point.

### FIELD OF THE INVENTION

An integrated process that combines Atmospheric Carbon Dioxide removal and sequestration using algae related technologies with the production and distribution of electrical power through conventional or Zero Emission methods and an underground Electric Pipeline along with the production, transportation and processing of oil, natural gas, renewable methane, renewable diesel, renewable gasoline, sustainable aviation fuel, salt water, fresh water, carbon dioxide and related coproducts and services. The process can eliminate all greenhouse gas emissions, while creating Emissions Free Energy, EFE, coproducts such as fresh water, ammonia fertilizers, plastics, chemicals, renewable energy resources, electricity, electrical energy storage, and an electrical driven transportation system.

### BACKGROUND OF THE INVENTION

The world is facing some severe challenges as we enter the second decade of the 21^{st} century. These challenges include shortages of food, fresh water and clean energy to provide for the growing global population. At the same time, research indicates that the global climate is changing as temperatures rise as a result of greenhouse gas emissions. While the fix for climate change seems to be the elimination of fossil fuels, this action would have a devastating effect on economies around the globe, leading to loss of jobs and reduction of food supplies. The question is, can we continue to use these plentiful fossil fuel resources in a way that does not lead to climate change, while increasing supplies of food, fresh water and clean energy, even reducing greenhouse gas levels in the atmosphere? The Zero Emissions Engine Processor, Electric Pipeline and Algae Related Technologies, ZEEP-EPART, concept does this and much more.

### SUMMARY OF THE INVENTION

The Zero Emissions Engine Processor, Electrical Pipeline and Algae Related Technologies, ZEEP-EPART, concept relates to new innovative ways of producing Emissions Free Energy, EFE, electrical power, storing it and distributing it to the surrounding areas and great distances, through the use of an innovative underground conductor and hydroelectric pump back waterway, along with the processing of saltwater, natural gas and various coproducts, all while eliminating harmful emissions and increasing operational efficiencies. By adding the Algae Related Technologies, which include methods for growing, processing, transportation and sequestration for over 100-years, atmospheric levels of CO2 can be reduced by Billions of Tons per year. All inputs for algae production and sequestration such as fresh water, electrical power and fertilizer can be supplied emissions free by the concept. The concept will be sustainable when the sequestered alga begins to produce sufficient methane to replace the natural gas used by the process. The methane along with other hydrocarbon production occurs naturally as heat and pressure increase in the formation. Methane from algae can also be produced by anerobic digestion or other methods such as torrification or heating in a pressurized vessel as described in patents filed by Dr. Phillip Savage.

Another process collects food waste from homes, restaurants, supermarkets, growing, storage and processing facilities, then converts it to methane by anerobic digestion or other method. Methane can either be used in the place of natural gas to produce electricity and heat or to produce renewable diesel, renewable gasoline, sustainable aviation fuel and other renewable hydrocarbons using Fischer-Tropsch or other processes.

Another process directly converts the captured CO2 into CO, which is combined with hydrogen to produce renewable diesel, renewable gasoline, sustainable aviation fuel and other renewable hydrocarbons using Fischer-Tropsch or other processes.

When applied to the Oil, Gas, Transportation and Electrical Production industries and their infrastructures, the ZEEP-EPART can virtually eliminate all greenhouse emissions from these industries while creating EFE coproducts such as fresh water, ammonia fertilizers, hydrogen, electrical energy storage, and an electrical driven transportation system. The oil and gas industry consumes a great deal of electrical energy for the production, processing and distribution of oil, gas and natural gas liquids. Typically, this electrical power is produced far from the oil and gas fields, and a great deal of energy loss is experienced. The gas is sometimes piped hundreds of miles from the source, used to produce electrical power, and then the electricity is run hundreds of miles back to produce and distribute the very same natural gas. Along with the oil and gas industry, the transportation infrastructure can be enhanced by the Electric Pipeline as it follows highways, railroads and waterways providing power for direct and indirect electrically induced mechanical energy to cars, trucks, railroad cars & locomotives and barges or other vessels. It also provides waterways with an electrical connection for operating hydroelectric pumps and generators for storage of and release of electrical power and movement of water from place to place.

The Zero Emissions concept comes from a previously disclosed invention called the Zero Emission Engine, ZEE, which was part of U.S. Pat. No. 10,465,491. It produces mechanical energy without creating harmful environmental emissions. The concept uses natural gas or other energy sources to produce hydrogen and combusts it with pure oxygen, driving turbines or turbo expanders to produce electricity and then recovers heat from the exhaust, producing pure water. The concept also works with natural gas or other fuel directly, by combusting it with pure oxygen, driving turbines or turbo expanders to produce electricity and then recovering heat from the exhaust, producing pure water and carbon dioxide, then recovering the carbon dioxide for use as an enhanced oil recovery, EOR, method, direct mineralization feedstock or by injecting it in deep underground saline formations. One aspect of using the CO2 as an EOR method is the possibility to produce shale oil and gas without fracking. This can be done by first opening the formation by drilling a series of horizontal holes using a novel electric or drilling fluid powered drill head attached to flexible tubing housed in a rigid coil tube from a tube coil rig. Then injecting a high-pressure continuous flow of CO2, saltwater and proppant mix from a centralized injection well toward the horizontal production wells that form a perimeter at a certain distance from the injection well.

A unique engine concept can be used to produce electricity from natural gas and oxygen with the products of combustion being directly compressed for water removal prior to injection underground. The concept reduces the cost of electricity by lowering the capital investment in engine, generator and carbon dioxide recovery components, while maintaining a zero emissions operation. This Emissions Free Energy, EFE, electricity is used to drive the local processes, and the rest can be exported to distant high value customers through a conventional or new innovative Electric Pipeline distribution system.

Another method of producing Hydrogen from Natural Gas is by Pyrolysis using a carbon plasma arc or high temperature to split the hydrogen and carbon. The carbon, which is in solid form, is used to make carbon electrodes, activated carbon or any other carbon product that does not oxidize the carbon. In this case, no CO2 is produced, and the Hydrogen is emissions free when used in electrical or coproduct production. A use for the activated carbon is as an absorbent for hydrogen, providing a method for vehicle use. It is estimated that an activated carbon block the size of a 1 cubic foot and weight of 20 pounds and could store enough hydrogen to power a vehicle for 50 miles.

The Electric Pipeline distribution method involves the use of innovative Calcium Underground Conductor, (CUC), or conventional above or below ground conductors that follow the pipeline network, highways, railroads, rivers and navigation channels. This Electric Pipeline concept allows for the increase in efficiency of Oil & Gas pipeline transportation, by increasing the quantity of pumps or compressors along the way, using conventional or innovative underground electrical driven equipment. It also supplies energy in areas where there is little or no current electrical distribution, such as the oil and gas production field. It decreases oil and gas exploration and production cost by replacing the diesel driven generation equipment. Compared to natural gas engine driven equipment, operating at 30% efficiency, the ZEE electrical power is produced at greater than 50% efficiency. This Electric Pipeline can also be used to supply other consumers in areas where there is little or no current electrical distribution. It can also be used to move this EFE power into a grid system for distribution around the country. The Electric Pipeline method also involves following highways, railroads and waterways, creating Electric Highways, Electric Railways and Electric Waterways. These will allow for the powering of electrical vehicles, (EVs), electric locomotives, (ELs), and electric marine vessels, (EMVs) through a direct wire transfer or by methods of power transfer presented in this patent application. Conventional highway vehicles can also be powered through a specially designed wheel or tire that is driven directly from the highway via an embedded magnetic drive system. Similarly, railroad locomotives or railcars can be powered through a specially designed wheel that is driven directly from the rails via an added magnetic drive system. The Electric Waterways can be used to store electrical energy in the form of hydroelectric pump back for upstream flow and release through generation with downstream flow. A novel hydroelectric pump/generator and lock design presented in this patent application would greatly reduce the cost of hydroelectric projects, allowing new waterways to move water upstream for energy storage or provide flood relief to downstream areas. One proposed project moves water from the Mississippi River over the continental divide to areas of the desert southwest. The Southwest Passage would provide water for the algae related technologies discussed later and the means for transfer of electrical power from remote solar and wind electrical production facilities to areas around the country.

Another feature of using the ZEE and ZEEP-EPART is the production and consumption of byproducts from these processes. For example, the nitrogen produced as a byproduct of oxygen production can be used to augment or replace the refrigerant system of a conventional natural gas processing plant. Liquid nitrogen can be used as the refrigerant for a modified Cryogenic Gas Plant. The resulting nitrogen refrigerant vapor can then be used in other processes or further heated and used to drive electrical production turbines or turboexpanders, further increasing electrical production. Finally, the nitrogen vapor can then be used for other processes such as the production of ammonia or use as an inert blanket gas for processes such as calcium metal production and calcium electrical conductor manufacture.

When using natural gas to produce hydrogen, pure water steam with high temperatures and pressures resulting from the ZEE, improve the efficiency and reduce the cost of hydrogen production. This allows for increased use of hydrogen as a feed stock for production of products such as ammonia fertilizers, hydrochloric acid and methanol. This makes it possible to have carbon neutral products to replace those that contribute to greenhouse gas emissions. The sequestration of carbon dioxide, CO2, is a vital part of the ZEE process. No other method is known to be more effective or advantageous than using it as an enhanced oil recovery, EOR, method. The CO2 can be piped the short distance to the oil production wells and injected deep underground permanently trapping it in the former oil-bearing formation. Any CO2 that finds its way back to the surface with the oil will be captured and returned to the formation until all oil is recovered and the wells are permanently cemented shut. This makes the ZEEP-EPART process truly a zero-emission method of producing mechanical energy, electricity, hydrogen, ammonia fertilizer, hydrochloric acid, methanol, calcium electrical conductors, cement, chlorine, polyethylene electrical conductor insulation, polyethylene pipe and various other products. If the CO2 EOR reaches one metric ton per barrel of oil produced, the oil produced could be considered CO2 emissions free, because the amount of CO2 emitted during processing and utilization will have already been sequestered in advance.

This concept is not limited to use in oil and gas production areas. The only requirement is a source of fuel such as natural gas or bio-methane and deep saline formations for water source and CO2 sequestration or saline water source when using direct mineralization. In the case of deep saline formations, the water removed for processing will create up to 4-times the pore space required for the CO2 sequestration. Direct mineralization requires elements such as sodium, calcium or magnesium to form the carbonates that will permanently sequester the CO2. Algae production can also be used to sequester the CO2. Depleted oil & gas wells can be used to sequester the algae produced, keeping the CO2 captured from the atmosphere from returning to the atmosphere for over 100-years. In this case, the methane or fuel production would be considered renewable when the algae are used to produce the methane or liquid fuel. One method for the conversion of algae to methane and liquid fuel is to deposit algae in vacated petroleum formations or underground caverns and allow time, heat and pressure to break down the algae into methane and other hydrocarbons. This renewable hydrocarbon process also preserves the nutrients and water for reuse in future algae growth. Saline formations can also be used to sequester the algae, while providing a saltwater source for conversion into freshwater and coproducts. This sequestered alga will eventually be transformed underground by heat and pressure into renewable oil and natural gas for future consumption as needed.

Algae production can be enhanced by using a novel Algae Production Panel, APP. The APP allows for any exterior flat or inclined surface to be used to produce algae. The APP is designed to allow for algae bearing water to be circulated in an enclosed space, using a metal or poly trough with a clear glass or poly film to allow sunlight to penetrate the shallow water promoting algae growth. Algae and water, along with nutrients are introduced at the feed end of a long panel, while oxygen and remaining CO2 is removed. CO2 is introduced to the outlet end of the APP, while the concentrated algae solution is removed. The oxygen and unused carbon dioxide can then be directed to the intake of an internal or open combustion engine, combined with fuel, and combusted to produce mechanical energy for driving electrical generators or other uses. The engine exhaust, rich in carbon dioxide, is then directed to the outlet end of the APP, resulting in a zero emissions process for electrical or mechanical energy production. Depending on the fuel type, the oxygen requirement for the engine and the carbon dioxide requirement for the algae production may or may not be in balance. Oxygen or carbon dioxide adjustments can be made from other sources. In the case of Natural Gas, when the usage is split between 50% electrical production and 50% hydrogen production, the oxygen production is in balance with the CO2 requirement of the Algae. Building roofs would be a great place to use the APP except during heavy rain or snow fall. In this case, the panel would be drained to ensure that the roof would not be overloaded. In the roof mounted APP, the panel could be made of poly film. The APP can also be used to remove CO2 from the atmosphere. Algae and water, along with emissions free nutrients are introduced at the feed end of a long panel, while nitrogen, oxygen and trace CO2 and other gases are returned to the atmosphere. Air is introduced to the outlet end of the APP, while the concentrated algae solution is removed for processing and storage. Since the oxygen concentration is higher coming out of the APP than it was when it entered, this gas stream could be used as a source of oxygen for combustion or processing.

Another Algae production method uses a lined pit to provide a body of water with flat surface for APP to float on. In this case the APP is a long tube partially filled with water made of clear flexible film. The tubes can be as long as necessary to reach across one dimension of the pit. The tubes can interconnect to form a continuous seal across the other dimension of the pit. This film barrier will prevent evaporation and provide an air space for the transmission of CO2. The algae would circulate through the tubes until optimum concentration is reached, then the algae could be released into the water below the film. Depending on the depth of the pit, the algae could settle to the bottom and build up over a period of years, then the algae could be removed for transport to an injection well or permanently impounded in place.

Another option for the APP is to have thin film solar cells affixed to the surface of the APP. This would convert some of the sunlight to electrical power, while still allowing sufficient solar energy to drive the algae growing process. This extra electrical power could drive some of the processes associated with the algae production, processing or sequestration, or stored to later power lighting to drive the algae production process during nighttime hours. Round the clock algae production would reduce the required growing area by up to 60%, or increase the algae produced in a given area by up to 60%.

Another option for the APP is to use concentrated solar energy from a trough lined with reflective material focused on the bottom portion of a clear or opaque pipe covered with high efficiency solar cells. The water containing algae, nutrients and CO2 circulates in the pipe keeping the solar cells cool to maintain efficiency. The clear pipes utilize the direct and reflected solar energy to maximize algae production and methods for tilting and rotating the trough to maximize overall solar intensity.

Another feature provides methods for the production of oil and natural gas from tight shale formations without fracking by using a novel drilling method to increase the opening in the formation then injecting a mixture of Saltwater, CO2 and some proppant to continuously push oil and natural gas from tight shale formations toward production wells. The steady pressure lifts the formation, allowing the mixture to reach the trapped hydrocarbons and freeing them. Once all hydrocarbons have been removed, CO2 is injected providing permanent sequestration.

### BRIEF DESCRIPTION OF THE DRAWINGS

Some embodiments are described with respect to the following figures:

FIG. 1a is a block flow diagram of an embodiment, the Zero Emissions Engine Processor, Electrical Pipeline and Algae Related Technologies, (ZEEP-EPART), that combines the production of electricity using various fuels and the treatment of saltwater along with the production of various coproducts all while capturing and sequestering the CO2 produced during combustion and directly from the atmosphere using Algae and other methods. It also provides methods for the movement of products in and out of the processor, including the distribution of electricity throughout the system and other markets using an innovative calcium underground conductor, CUC. Coproducts include Fresh Water, Nitrogen, Sodium Chloride, Calcium Chloride, Chlorine, Sodium Hydroxide (NAOH), Hydrochloric Acid, Calcium, Calcium Oxide and Carbonates of Calcium, Sodium, Magnesium and Other Elements.

FIG. 1b is a schematic of the algae processing opportunities within the ZEEP-EPART.

FIG. 2a is a block flow diagram of an embodiment, the Zero Emissions Engine Processor, Electrical Pipeline and Algae Related Technologies, (ZEEP-EPART), given in FIG. 1 adding Hydrogen production by Steam Reformer & Water Shift along with Ammonia production for internal and external use.

FIG. 2b is a block flow diagram of an embodiment, the Zero Emissions Engine Processor, Electrical Pipeline and Algae Related Technologies, (ZEEP-EPART), given in FIG. 1 adding Hydrogen production by High Temperature or Plasma Arc Pyrolysis along with Ammonia production for internal and external use.

FIG. 3a is a block flow diagram of an embodiment, the Zero Emissions Engine Processor, Electrical Pipeline and Algae Related Technologies, (ZEEP-EPART), given in FIG. 1 adding Liquid Oxygen and Nitrogen, Hydrogen production by Steam Reformer & Water Shift and Ammonia production along with Wet Natural Gas Processing. This arrangement is suited for location in the Oil and Gas Production areas. Additional coproducts such as Ethylene, High Density Polyethylene, Vinyl Chloride and Polyvinylchloride along with other products related to a cryogenic Natural Gas Plant. This co-location with a gas plant allows for use of nearly pure methane as fuel for the zero emissions engine and feedstock for Hydrogen production, allowing higher value ethane to be processed into ethylene or piped to other markets. This could improve the profitability of Electrical, Hydrogen and Ammonia production.

FIG. 3b is a block flow diagram of an embodiment, the Zero Emissions Engine Processor, Electrical Pipeline and Algae Related Technologies, (ZEEP-EPART), given in FIG. 3a changing to Hydrogen production by High Temperature or Plasma Arc Pyrolysis.

FIG. 4 is a block flow diagram of an embodiment, the Zero Emissions Engine Processor, Electrical Pipeline and Algae Related Technologies, (ZEEP-EPART), given in FIG. 3 replacing the Methane fuel with Hydrogen fuel. The co-location with a gas plant allows for use of nearly pure methane as a feedstock for hydrogen production, allowing higher value ethane to be processed into ethylene or piped to other markets. This could improve the profitability of hydrogen production. The use of hydrogen as fuel for heat and electrical power production allows the capture of CO2 to take place in one area and the pure steam exhaust from the zero emissions engine to be used as a process feedstock for other processes. Hydrogen can be produced from methane by either Steam Reformer & Water Shift or High Temperature Pyrolysis.

FIG. 5 is a block flow diagram of an embodiment, the Zero Emissions Engine Processor, Electrical Pipeline and Algae Related Technologies, (ZEEP-EPART), given in FIG. 1 showing a retrofit to an existing Gas Turbine or Reciprocating Engine power production installation. The existing components are red and outlined by dotted line.

FIG. 6 is a block flow diagram of an embodiment, the Zero Emissions Engine Processor, Electrical Pipeline and Algae Related Technologies, (ZEEP-EPART), given in FIG. 1 showing a retrofit to an existing Combined Cycle Gas Turbine Electrical Power production installation. The existing components are red and outlined by dotted line.

FIG. 7 is a block flow diagram of an embodiment, the Zero Emissions Engine Processor, Electrical Pipeline and Algae Related Technologies, (ZEEP-EPART), given in FIG. 1 showing a retrofit to an existing Coal Fired Electrical Power Plant. The existing components are red and outlined by dotted line.

FIG. 8a is a detailed schematic depiction of the Zero Emissions Engine Processor, ZEEP, described in the block flow diagram given in FIG. 1.

FIG. 8b is a detailed schematic depiction of the Zero Emissions Engine Processor, ZEEP, described in the block flow diagram given in FIG. 1 with the addition of hydrogen production using methane pyrolysis.

FIG. 9a is a detailed schematic depiction of the Zero Emissions Engine Processor, ZEEP, described in the block flow diagram given in FIG. 2a.

FIG. 9b is a detailed schematic depiction of the Zero Emissions Engine Processor, ZEEP, given in block flow diagram FIG. 2b.

FIG. 10 is a detailed schematic depiction of the Zero Emissions Engine Processor, ZEEP, given in block flow diagram FIG. 3a.

FIG. 11 is a detailed schematic depiction of the Zero Emissions Engine Processor, ZEEP, given in block flow diagram FIG. 3b.

FIG. 12 is a graphic depiction of an expansion of the system depicted in FIGS. 8-11 with the focus being a unique Zero Emissions Engine concept, using pressurized air, oxygen or liquid oxygen combined with natural gas or other fuel that produces electrical power directly by the movement of a magnetic piston through magnetic fields associated with an internal combustion engine. It also incorporates exhaust compression along with nitrogen and water separation functions to provide pressurized Carbon Dioxide for EOR or other sequestration method. Two designs are presented, Option 3A a single double acting piston in a single cylinder and Option 3B using two single or double acting pistons in two cylinders interconnected by a connecting rod, operating in either two or four stroke configurations. Option 3A is the simplest design, but efficiencies are lower due to heating of magnets and coils. Option 3B removes the magnets and coils from the heat effected zone and allows for increased numbers of magnets and coils.

FIG. 13 is a schematic depiction of the graphic system depicted in FIG. 12, with allowances for CO2 separation from nitrogen when air is used rather than oxygen.

FIG. 14 is a graphic depiction of an expansion of the system depicted in FIG. 11 with the focus a unique Zero Emissions Engine concept, using oxygen or air combined with hydrogen as fuel in a high-pressure vessel with process connections to produce hydrogen.

FIG. 15 is a graphic depiction of an expansion of the system depicted in FIG. 11 with the focus a unique Zero Emissions Engine concept, using oxygen or air combined with hydrogen as fuel in a high-pressure vessel with Heat Exchanger for Pyrolysis production of hydrogen and carbon from Natural Gas along with process connections. It also includes a method to store hydrogen for mobile use in activated carbon, which provides a use for the carbon that is by the pyrolysis of methane.

FIG. 16 is a schematic depiction of the graphic system depicted in FIG. 14.

FIG. 17 is a schematic depiction of the embodiment depicted in FIG. 10 with the focus on the integration of the gas processing system using liquid nitrogen as a refrigerant.

FIG. 18 is a schematic depiction of the embodiment depicted in FIG. 17 with the focus on the integration of the ethane production using liquid nitrogen as a refrigerant for the further production of ethylene, High Density Polyethylene, (HDPE), and polyvinylchloride, (PCV), to be used in sheathing of the calcium conductors and for manufacturing of pipe used in oil and gas gathering and other industries.

FIG. 19 is a schematic depiction of the embodiment depicted in FIG. 18 with the focus on the integration of the ethylene production using CO2 as a soft catalyst or electrochemical process using a catalyst.

FIG. 20 gives a cross section the corridors that move products to and from the well pads along with corridors that move products to and from pump and compressor stations. It also describes the well layout of a 1,600 square mile production zone with CO2 Injection in the center pushing oil and natural gas outward and moving the injection points outward as wells are depleted. The layout can be used to inject algae for CO2 sequestration which would convert the depleted formation back into an eventual renewable oil and gas production zone. This can be accomplished by forcing concentrated algae into the center most injection well after the CO2 injection has been switched to an outer ring of injection wells that were converted from producing wells after CO2 flow through reaches a set level.

FIG. 21 is a graphic depiction of an expansion of the system depicted in FIG. 20 with the focus on the movement of products, including oil, wet gas, dry gas, produced water, fresh water, proppant, NGLs, CO2, Algae, LNG and electrical power into and out of the oil and gas production zone.

FIG. 22 is a block diagram of the embodiment depicted in FIG. 20 with the focus on the step-by-step process from drilling initial well to the transformation of the well to a renewable oil and gas producer.

FIG. 23 is a schematic depiction of an Oil and natural gas production zone that uses the position of the wells to provide enhance hydrocarbon production by injecting CO2 or mixture of CO2 and water to provide continuous washing of the hydrocarbons from the tight or loose formation toward the producing wells. This maximizes the hydrocarbon production and eventually fills the original pore space with CO2 for permanent sequestration. This method could be used to produce tight shale formations without fracking. The layout can also be used to inject algae for CO2 sequestration which would convert the depleted formation back into an eventual renewable oil and gas production zone.

FIG. 24 shows a concept for a frackless Coil Tub Rig with Micro-Drilling operation for tight oil and gas formations using horizontal drilling. This method requires a standard horizontal well be drilled. Then a coil rig 480 would be brought in with a Novel Flexible Tube, NFT, 481 that can allow for tighter turns, but performs similar to a standard drilling pipe. Also included are the Micro Drilling Head, MDH, 482 and an Indexing Micro Drill Guide, IMDG, 483, that positions the MDH, 482. First the IMDG, 483, is positioned in the predetermined position in the horizontal section of the well bore. This IMDG, 483 can be an electrically powered device that pulls itself into position when attached to the NFT, 481, pr a passive device attached to a standard coil tube. The IMDG, 483 can rotate and index to different angles of the 360 degree well bore, allowing for multiple small bores to be drilled along the horizontal bore allowing for more formation exposure as well as pore space and flow channels. The Micro Drilling Head, MDH, 482, is then sent down the well bore to IMDG, 483, and the self-powered mud or electric device begins drilling through the casing to a predetermined length and angle. The NFT, 481, can have assisted propulsion both forward and backward through electric tether and NFT, 481, to wall interface. Once the final distance has been achieved, the NFT, 481, and MDH, 482 are retracted past the IMDG, 483, and the IMDG, 483, is indexed to the next position. When all holes are drilled at the IMDG, 483, position, the IMDG, 483, is moved to the next position along the horizontal bore.

FIG. 25 shows an enlarged view of the horizontal Micro Drill concept.

FIG. 26 shows a concept for a frackless Coil Tub Rig with Micro-Drilling operation for tight oil and gas formations using a Vertical Well. This method requires a standard verticle well be drilled. Then a coil rig 480 would be brought in with a Novel Flexible Tube, NFT, 481 that can allow for tighter turns, but performs similar to a standard drilling pipe. Also included are the Micro Drilling Head, MDH, 482 and an Indexing Micro Drill Guide, IMDG, 483, that positions the MDH, 482. First the IMDG, 483, is positioned in the predetermined position in the vertical section of the well bore. This IMDG, 483 can be an electrically powered device that pulls itself into position when attached to the NFT, 481, pr a passive device attached to a standard coil tube. The IMDG, 483 can rotate and index to different angles of the 360 degree well bore, allowing for multiple small bores to be drilled along the vertical bore allowing for more formation exposure as well as pore space and flow channels. The Micro Drilling Head, MDH, 482, is then sent down the well bore to IMDG, 483, and the self-powered mud or electric device begins drilling through the casing to a predetermined length and angle. The NFT, 481, can have assisted propulsion both forward and backward through electric tether and NFT, 481, to wall interface. Once the final distance has been achieved, the NFT, 481, and MDH, 482 are retracted past the IMDG, 483, and the IMDG, 483, is indexed to the next position. When all holes are drilled at the IMDG, 483, position, the IMDG, 483, is moved to the next position along the vertical bore.

FIG. 27 shows an enlarged view of the vertical Micro Drill concept.

FIG. 28 is a schematic depiction of the embodiment depicted in FIG. 3 focusing on a process for producing calcium metal and its utilization in the manufacture of a new underground electrical conductor. This calcium conductor is surface treated with nitrogen or aluminum, then sealed inside a polymer casing such as High-Density Polyethylene, (HDPE), which will provide a low-cost alternative to copper and aluminum wire.

FIG. 29 is a schematic depiction of the embodiment depicted in FIG. 19 with the addition of a process for the interconnection of calcium conductors, making a continuous conductor over long distance.

FIG. 30 is a block flow diagram of an embodiment, the Electric Pipeline, EP, that details the distribution of electricity throughout the grid system and other markets including Electric Highways, Railroads and Waterways using the innovative calcium underground conductor, CUC, or conventional above and below ground conductors.

FIG. 31 is a schematic depiction of a proposed method of transmission of electrical power depicted in FIG. 30 using two DC high voltage conductors with medium voltage differential between conductors plus a third conductor to act as a neutral. An electronic inverter will create medium AC voltage or pulse current anywhere along the corridor providing power for conventional motor or advanced linear motor driven equipment.

FIG. 32 is a schematic depiction of an expansion of the system depicted in FIG. 21 with the focus on a method for movement of electricity along highways while providing methods for moving vehicles along with the focus on the transfer of energy to non-electric powered vehicles through modified wheel to road interface.

FIG. 33 is a schematic depiction of an expansion of the system depicted in FIG. 29 the transfer of electrical power to electric vehicles when stationary or moving through a flywheel arrangement located in the wheel area of the vehicle.

FIG. 34 is a schematic depiction of an expansion of the system depicted in FIG. 29 with the focus on a vehicle with an addition of skates that interface with the road through coil mechanisms.

FIG. 35 shows additional details of the road to vehicle interface and a method of supplying DC power to an electric vehicle to charge or power the it as it moves down the road or is parked.

FIG. 36 is a schematic depiction of an expansion of the system depicted in FIG. 21 with the focus on a method for movement of electricity along railroads while providing methods for transfer of electrical power to electric powered railroad locomotives or railcars to provide power for the movement of the train. It also shows a schematic depiction of an expansion of the system depicted with the focus on the transfer of energy to railcars through modified shoe to third rail interface. The system also allows the ability to provide motive force direct to railcars providing power for the movement of each individual railcar.

FIG. 37 is a schematic depiction of a system that provides electrical power to an electric locomotive that matches the precise voltage and frequency to match the speed, load, grade and resistance of the train through a 5G wireless connection. This eliminates the equipment on the locomotive like inverters, transformer and other control devices. Power can re-enter the grid when the train applies dynamic braking.

FIG. 38 is a schematic depiction of an expansion of the system depicted in FIG. 21 with the focus on a method for movement of electricity along waterways while providing methods for storage of electrical power for use during on-peak times using a hydroelectric pump back and generation system.

FIG. 39 is a schematic depiction of an expansion of the system depicted in FIG. 35 with the focus on the lock and channel design.

FIG. 40 is a schematic depiction of an expansion of the system depicted in FIG. 36 with the focus on the lock and channel details.

FIG. 41 is a schematic depiction of an expansion of the system depicted in FIG. 35 with the focus on the dam and channel design.

FIG. 42 is a schematic depiction of an expansion of the system depicted in FIG. 35 with the focus on an alternative dam interior.

FIG. 43 is a schematic depiction of an expansion of the waterway system depicted in FIG. 38 with the focus on the transfer of electrical power to marine vessels providing power for the movement of the vessel along the waterway either through propeller, cable draw or linear water propulsion system.

FIG. 44 is a schematic depiction of the embodiment depicted in FIG. 21 with the focus on pumping of oil, NGLs, water and CO2 and compression of natural gas. Electricity following the pipelines makes the position of pumps and compressors very versatile and can lower installation and operation costs. It also includes the interconnection with equipment such as pumps and compressors without penetrating the surface of the ground. Various pipelines delivering the products to market, combining the electrical transmission with the pipelines and the compressor and pump locations being optimized for terrain, pipe size and wall thickness, providing for a low environmental impact and lowest installation and operation cost. It also presents a variable speed function for motors driving pumps and compressors.

FIG. 45 is a schematic depiction of the embodiment depicted in FIG. 21 with a new concept pump and compressor concept allowing for components to stay underground and utilize electrical energy from the electric pipeline.

FIG. 46 depicts a system for growing algae in channels with thin film on water to reduce evaporation along with methods for supplying air, water, fertilizer, CO2 and algae remains in channel.

FIG. 47 is a depiction of the algae of algae growing panels on slopped or flat roofs or on the ground.

FIG. 48 is a depiction of the algae growing panels on land.

FIG. 49 is a depiction of the algae growing panels on lakes, seas or oceans.

FIG. 50 is a depiction of the algae panel on channels with solar cells to generate electrical power while shading algae from intense sunlight. Options for LED lights for nighttime growing.

FIG. 51 is a depiction of an algae panel that uses a trough to focus sunlight along a thin concentrated solar strip on the bottom of a clear glass or poly tube. Clear tube allows algae to grow using direct and scattered sunlight while cooling the concentrated solar cells.

FIG. 52 is a depiction of an algae sequestration method for algae growing system given in FIG. 46.

FIG. 53 is a depiction of the algae sequestration in deep saline formations or depleted oil-bearing formation with algae being slowly converted to methane and other hydrocarbons resulting in renewable oil and natural gas.

FIG. 54 is a depiction of the algae sequestration in depleted oil and gas formations, Saline Formation or Salt Cavern.

### DETAILED DESCRIPTION OF THE PREFERRED EMBODIMENTS

An integrated process that reduces carbon dioxide, CO2, and other harmful emissions in energy and chemical processes and reduces atmospheric concentration of CO2, all while increasing revenues and reducing costs. This will resolve energy, food and climate issues without causing negative impacts such as increased costs, food shortages or transportation disruptions. The Zero Emissions Engine Processor, Electric Pipeline and Algae Related Technologies, ZEEP-EPART, modifies the methods by which energy is produced, transported and utilized, resulting in truly Emission Free Energy, EFE, crude oil, natural gas, renewable diesel, renewable gasoline, sustainable aviation fuel, mechanical energy, electricity, hydrogen, ammonia fertilizer, hydrochloric acid, methanol, calcium electrical conductors, cement, chlorine, ethylene, polyethylene, propylene, polypropylene, polyvinylchloride, and various other products, permanently locking the resulting carbon dioxide and other harmful emissions deep underground. It includes provisions for the production of oil and natural gas from tight shale formations without fracking, the production of Renewable methane from algae, food waste, agricultural waste through anerobic digestion or other methods, replacement of coal with algae biochar or carbon from methane pyrolysis, improvement of
electrical energy production, transmission and utilization through Novel Zero Emissions Engine, Novel Underground Calcium Conductors and Electric Pipeline, Vehicles Powered by Novel Electric Road Interface, Trains Powered by Novel Electric Rail Interface, Marine Vessels Powered by Novel Electric Waterway Interface, Novel Algae Production, Novel Algae Long Term Sequestration Creating Renewable Oil and Natural Gas, Novel Algae Based Energy Production, Novel Fresh Water Production and Novel Fresh Water Conservation and Massive Electrical Energy Storage System through a Novel Electric Waterway Project (The Southwest Passage).

The Zero Emissions Engine Processor and Electrical Pipeline, ZEEP-EPART, concept is an integrated process that combines the conventional or Zero Emission production and distribution of electrical power with the production, transportation and processing of oil, natural gas, water, carbon dioxide and related products and services.

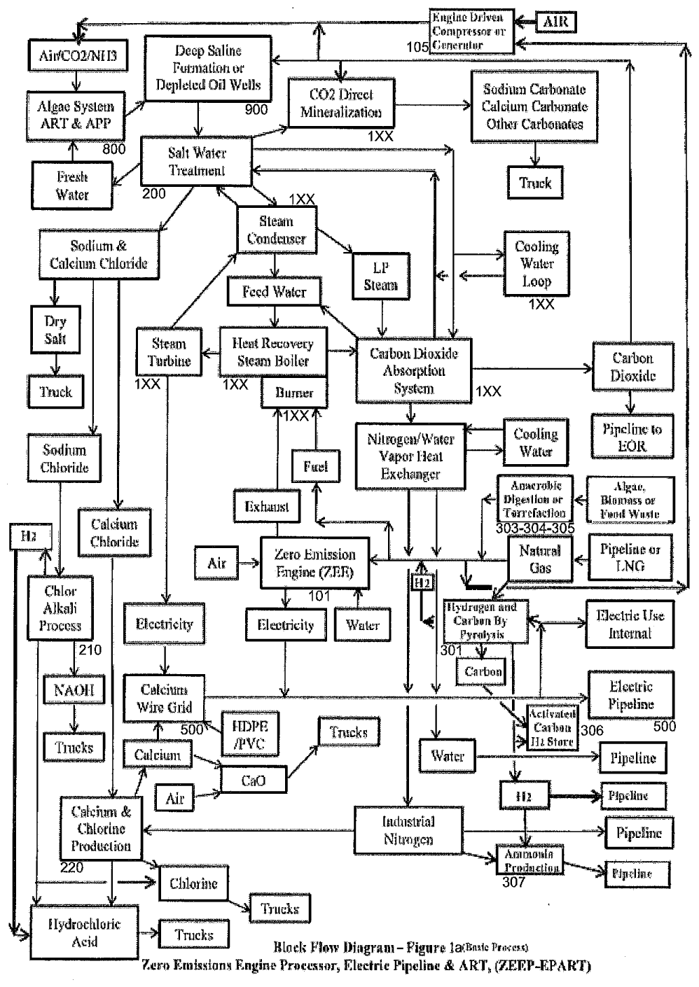

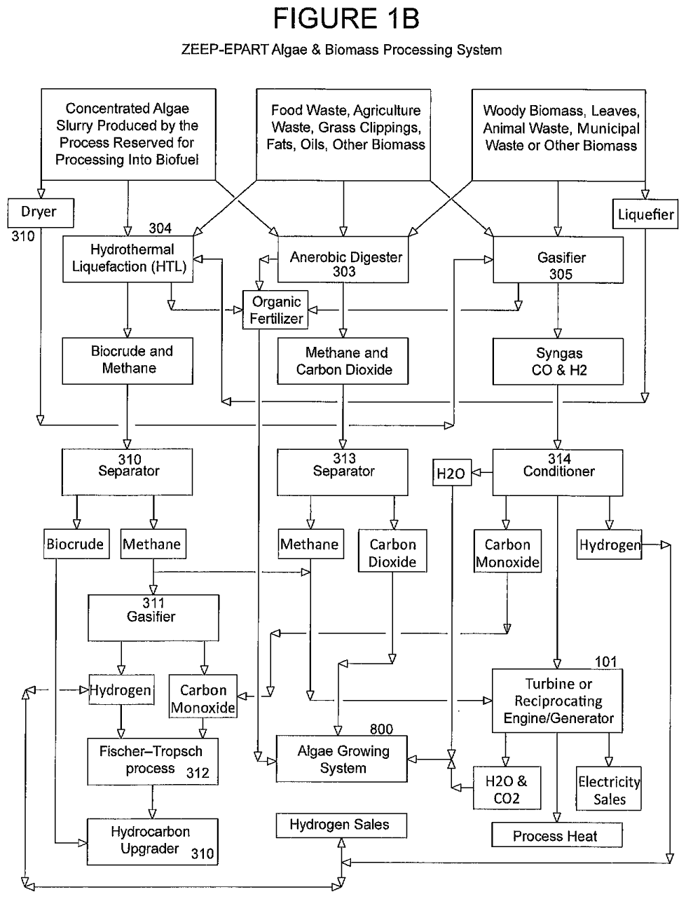

The Block Flow Diagram in FIG. 1a shows the interaction of the various components of the Zero Emissions Engine Processor, Electrical Pipeline and Algae Related Technologies, (ZEEP-EPART). Natural gas, biomethane or other fuel gas is combined with air, producing heat and converting it to mechanical energy in an internal combustion, gas turbine or other type engine. The engine output drives an electrical generator or other equipment such as compressor or pump. In the case of the gas turbine engine, the oxygen rich exhaust is directed to a reheat boiler, where additional fuel is added to consume the excess oxygen. The injection of water after combustion can reduce the amount of excess air required, reducing the excess oxygen in the exhaust stream. The reheat boiler boils water or other liquid to produce high pressure steam, which drives a steam turbine and additional electric generator or another device. CO2 rich exhaust is then directed to a carbon dioxide recover system, which separates the CO2 from Nitrogen and other vapors. The CO2 is then compressed, cooled and directed to a pipeline to be used as an enhanced oil recovery, EOR, solvent or to a saline well for sequestration. Once all recoverable oil is produced in the EOR formation, the formation is packed with additional CO2 until the maximum permanent storage pressure is reached. The carbon dioxide recover system is operated using cooling water from the saltwater treatment system, along with the heat contained in the exhaust, heat from low pressure steam and electricity. The nitrogen and water vapor leaves the carbon dioxide recover system and enters a heat exchanger where water is removed for internal or external use and the nitrogen is compressed and piped to internal or external processes. The Saltwater Treatment System receives saltwater from a Deep Saline Well or other source. It uses waste heat from the ZEEP to evaporate the water through an evaporative cooler that provides cooling water for the entire process. The water is then further evaporated in multi effect evaporators, raising the concentration to a high level. A flash evaporator utilizing the heat recovery boiler exhaust evaporates the remaining water, leaving dry metals, salts and compounds. These compounds, mainly sodium and calcium chlorides are further processed into valuable commodities or transported for use by others. Some carbonates are formed when the concentrated saltwater is exposed to the CO2 in the exhaust. The Sodium Chloride can be converted in a Chlor Alkali Process into Sodium Hydroxide, (NAOH), Hydrogen and Chlorine. Hydrogen and Chlorine can be combined to produce Hydrochloric acid. The Calcium Chloride can be processed into Calcium and Chlorine in a process described later. The Chlorine can be transported off site or converted to other products. The Calcium metal can be used to produce electrical conductors or reacted with air or oxygen to produce calcium oxide (CaO) or other products. The electrical conductors can be surface treated with nitrogen or aluminum to reduce risk of corrosion before being jacketed with High Density Polyethylene, HDPE or Poly Vinyl Chloride, PVC. This Calcium Conductor could be a low-cost replacement for the aluminum and copper conductors currently used allowing larger conductors to be placed underground running along highways, railroads and waterways, forming the backbone of an innovative Electric Pipeline. This electric pipeline will have features to provide direct interface with highway vehicles, train locomotives and other transportation vehicles along with the movement and storage of water and electricity through a canal system and hydroelectric pumps/generators. More details of Electric Pipeline, EP, will be provided in later drawings. The fresh water produced in the saltwater treatment system can be used to grow algae in an Algae Production Panel, APP, discussed later. The algae are also fed CO2 and Ammonia based fertilizer NH3. The algae can be produced at an annual rate of up to 100 metric tons per acre. These 100 tons of algae could capture up to 147 metric tons of CO2 from either the atmosphere or that captured by the process. These algae can be used as a food or energy source or sequestered in the deep saline wells or depleted oil wells. The algae could also be sequestered by impoundment in a long-term storage pit. In either case, the algae will slowly break down to form renewable hydrocarbons such as methane and heavier chains and can be utilized as a renewable fuel source as needed. The current oil and gas production in the Permian Basin would allow for the growing and sequestering of algae with ample sunlight along with low land and transportation costs. The advantage of using the Algae Related Technologies, ART, presented in this document is that dewatering of the algae is not required. A slurry of water and algae can be transported by pipeline or other method the short distance to the deep saline or depleted oil well and forced with high pressure into the surrounding formation.

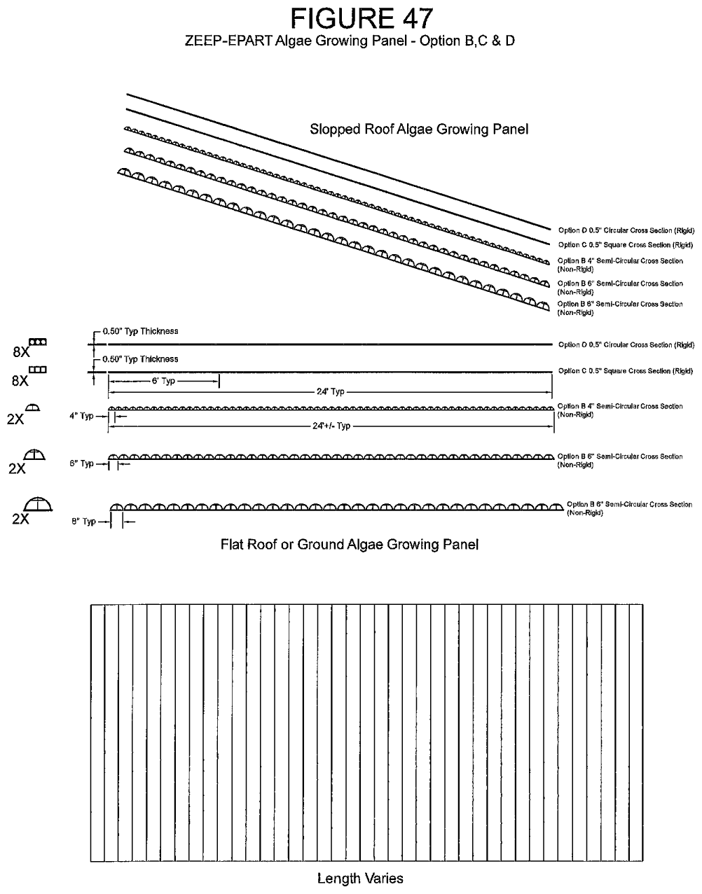

FIG. 1b is a schematic of the algae processing opportunities within the ZEEP-EPART. The algae processing has many possibilities to produce renewable fuels. Renewable methane is the most readily available in product from processing. It commands a good price and can produce cash flow for eventual shift to more valuable liquid fuels offsetting diesel gasoline in aviation view uses. The proposed system defined in FIG. 47 users three times value biomass. Algae food waste agricultural waste grass clippings fats and oils and other biomass which can be used in an aerobic anaerobic digester or hydrothermal liquification same goes with algae can be used in the hydrothermal liquification unit producing methane and biocrude in the anaerobic digester the biomass is converted into CO2 and methane finally the other biomass is woody biomass leaves animal waste municipal waste and other biomass which can be gasified producing a syngas. This sin gas can be a feedstock for the production of liquid fuels using the Fisher Tropes method of converting syngas to hydrocarbons. Syngas can also be used to power reciprocating engines producing electricity from low-cost feedstock.

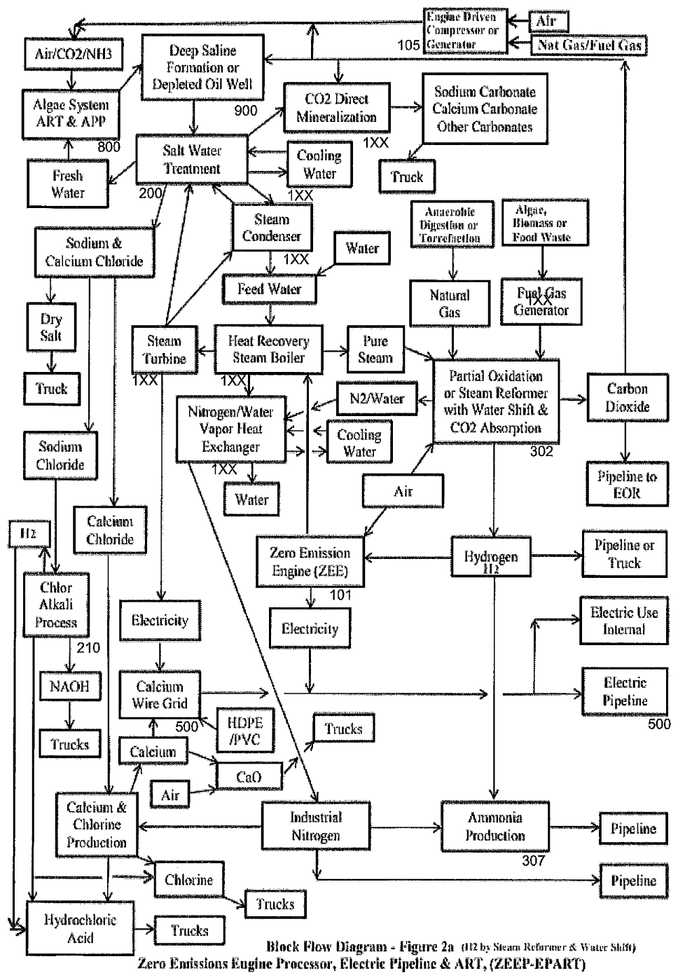

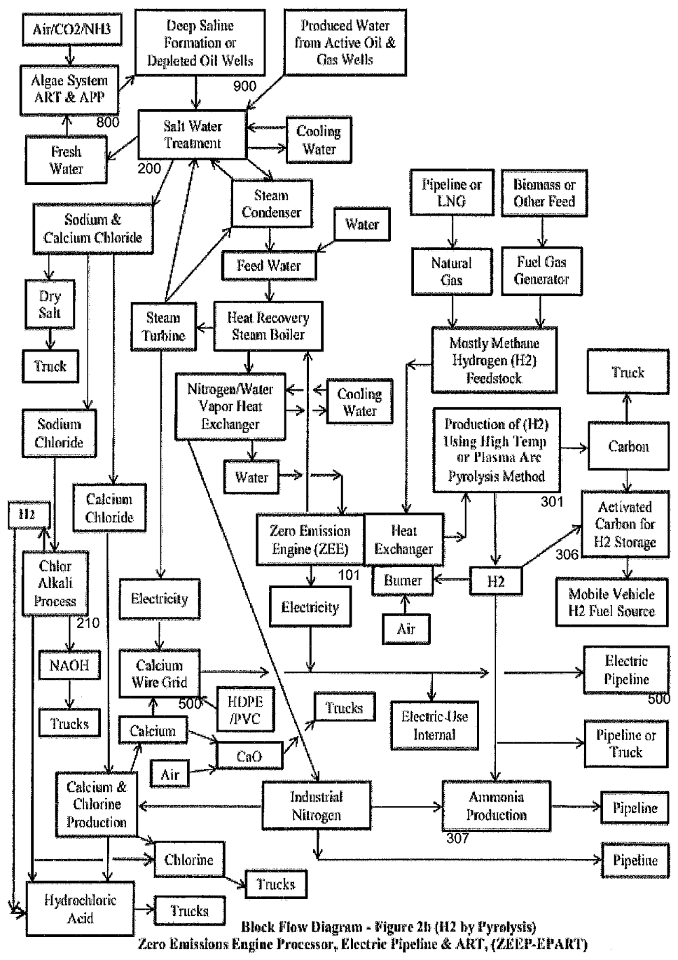

The Block Flow Diagram in FIG. 2a shows the same interaction of the various components of the Zero Emissions Engine Processor, Electrical Pipeline and Algae Related Technologies, (ZEEP-EPART) shown in FIG. 1 with the fuel to the engine being hydrogen and the Natural gas, biomethane or other fuel gas being directed to a Partial Oxidation or Steam Reformer with Water Shift and CO2 Absorption. In this case hydrogen is produced from the fuel gas under pressure allowing for the capture of CO2 to be performed more efficiently. The engine fueled by hydrogen has little or no CO2 in the exhaust stream, allowing for simplified water and nitrogen separation. Since excess hydrogen can be produced, additional components can be added such an ammonia production, which combines the Hydrogen with the already present Nitrogen. This becomes a feedstock for the Algae in the ART, and provides product revenue from emissions free Ammonia, NH3.

The Block Flow Diagram in FIG. 2b shows the same interaction of the various components of the Zero Emissions Engine Processor, Electrical Pipeline and Algae Related Technologies, (ZEEP-EPART) shown in FIG. 2A with the fuel to the engine being hydrogen and the Natural gas, biomethane or other fuel gas being directed to a Hydrogen High Temperature Pyrolysis Processor rather than the Partial Oxidation or Steam Reformer with Water Shift and CO2 Absorption.

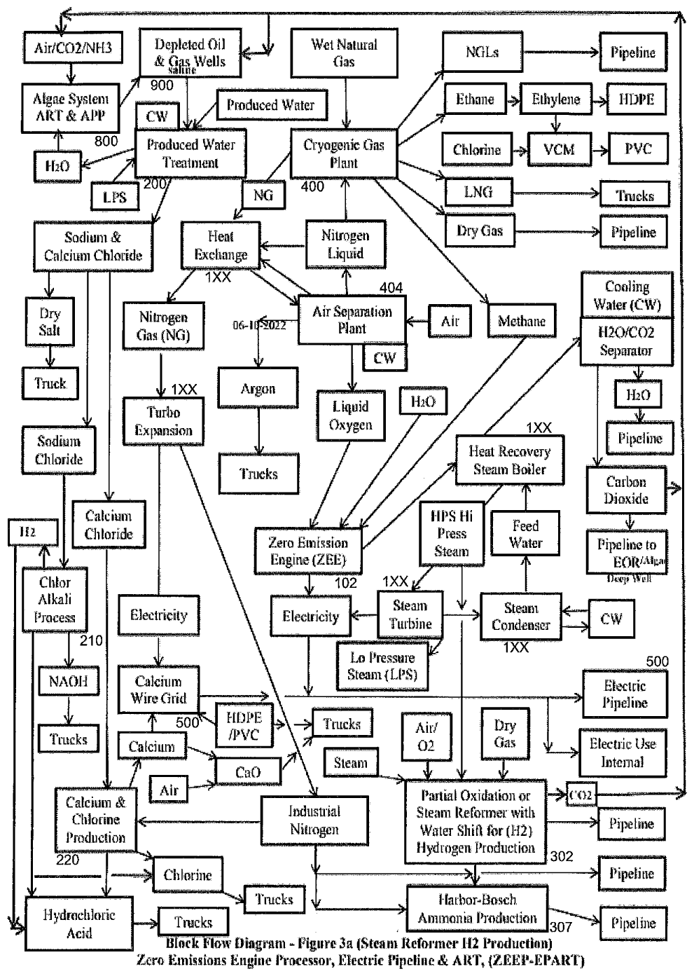

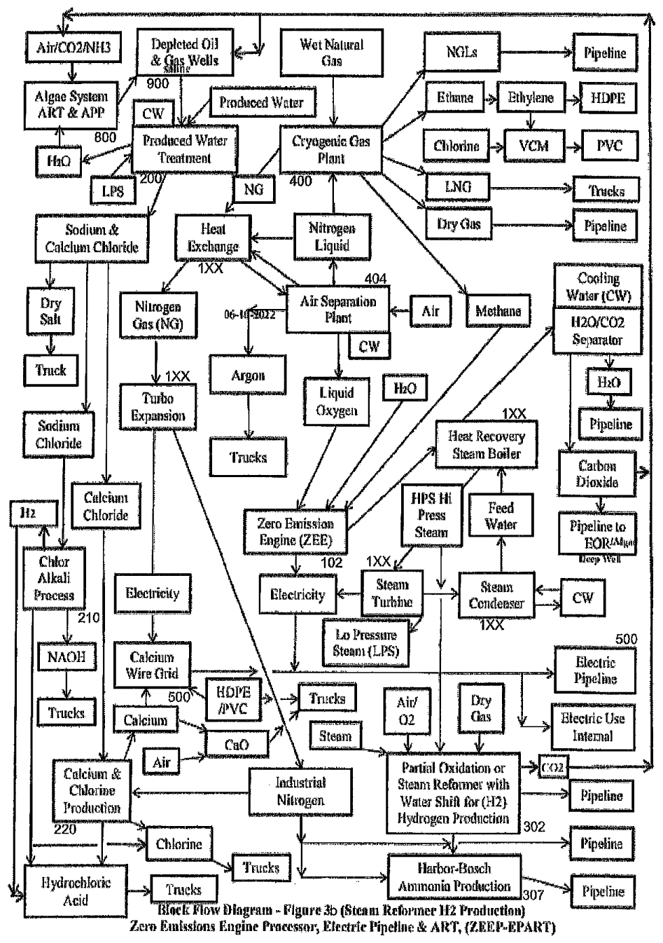

The Block Flow Diagram in FIG. 3a shows the same interaction of the various components of the Zero Emissions Engine Processor, Electrical Pipeline and Algae Related Technologies, (ZEEP-EPART) shown in FIG. 2 with the addition of a Cryogenic Gas Plant and an Air Separation Plant. The presents of the Gas and Air Separation Plants allow for combining the efficiency gains from the use of liquid Nitrogen as a refrigerant with the production of pure methane to be used for fuel for the engine and feedstock for Hydrogen production. Other feedstocks for the Calcium Conductors such as HDPE and PVC can be produced locally. The pure oxygen combined with Methane in the engine produces only water and CO2 in the exhaust, simplifying the exhaust treatment and eliminating the power required for air compression. Additional products such as Argon, Dry Natural Gas, Ethane, Ethylene, Polyethylene, High Density Polyethylene, (HDPE), Polyvinylchloride, PVC, Natural Gas Liquids NGL, Liquid Natural Gas, (LNG) and Pure Water can generate additional revenue. The liquid Nitrogen can be further heated after vaporization to higher temperatures, then expanded through turbine or turbo expander to produce additional electrical power.

The Block Flow Diagram in FIG. 3b shows the same interaction of the various components of the Zero Emissions Engine Processor, Electrical Pipeline and Algae Related Technologies, (ZEEP-EPART) shown in FIG. 3A with a Hydrogen High Temperature Pyrolysis Processor replacing the Partial Oxidation or Steam Reformer with Water Shift and CO2 Absorption.

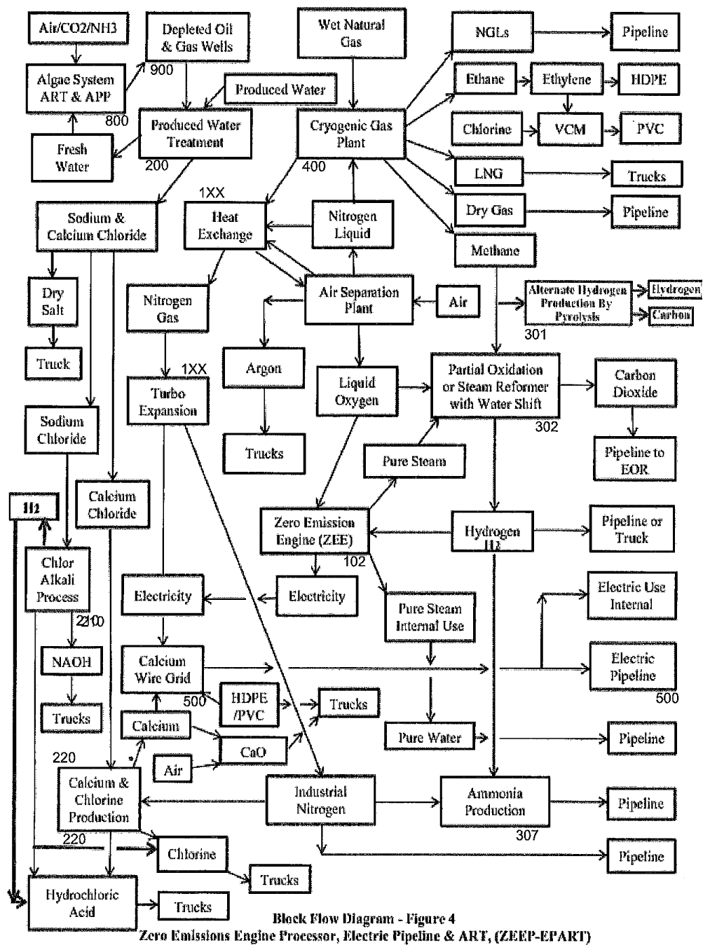

The Block Flow Diagram in FIG. 4 shows the same interaction of the various components of the Zero Emissions Engine Processor, Electrical Pipeline and Algae Related Technologies, (ZEEP-EPART) shown in FIG. 3 with some of the hydrogen being used as fueling the Zero Emissions Engine, ZEE. The ZEE could be operated under high pressure and temperatures, increasing efficiency and producing pure steam as an exhaust. The Hydrogen production could also be incorporated into the ZEE, taking advantage of the high pressure, temperature and high-pressure steam.

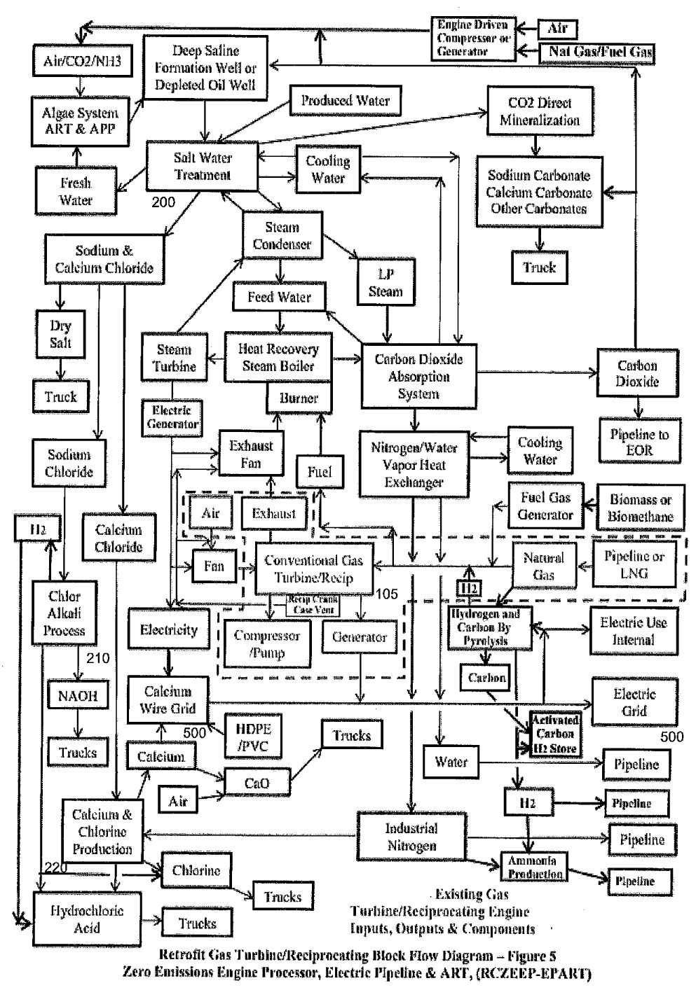

The Block Flow Diagram in FIG. 5 shows the same interaction of the various components of the Zero Emissions Engine Processor, Electrical Pipeline and Algae Related Technologies, (ZEEP-EPART) shown in FIG. 1 with the system retrofitting an existing Conventional Gas Turbine or Reciprocating Engine. The dotted line encompasses the existing equipment shown in red text. Additional components such as intake and exhaust fans will ensure that existing equipment continues to operate under optimum conditions. This retrofit converts the existing system to a Zero Emissions Engine Processor, ZEEP.

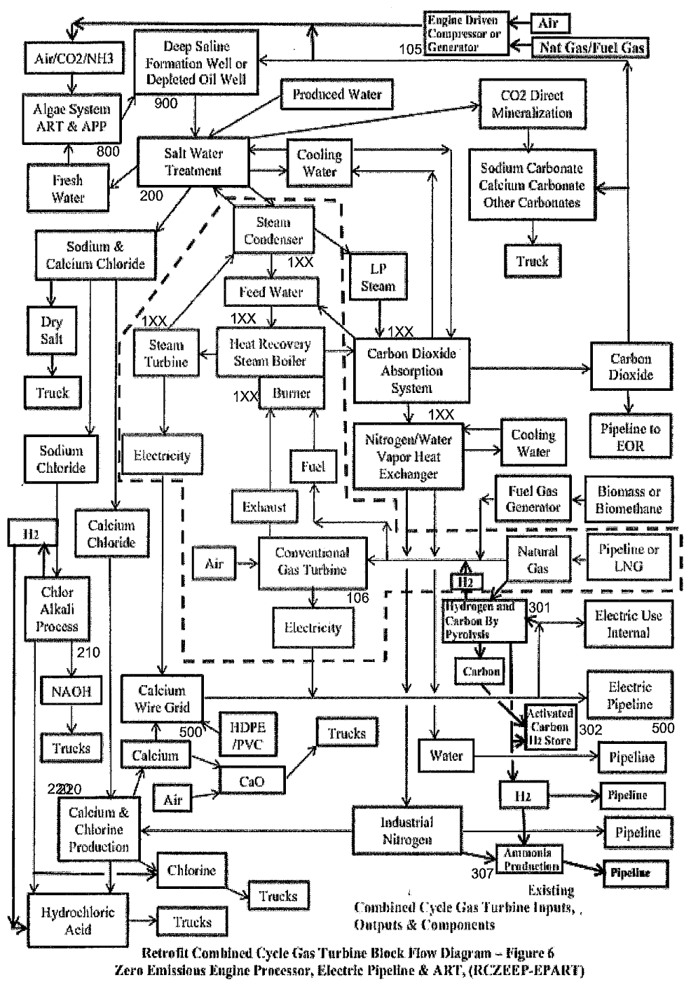

The Block Flow Diagram in FIG. 6 shows the same interaction of the various components of the Zero Emissions Engine Processor, Electrical Pipeline and Algae Related Technologies, (ZEEP-EPART) shown in FIG. 1 with the system retrofitting an existing Conventional Combined Cycle Gas Turbine. The dotted line encompasses the existing equipment shown in red text. Additional components such as intake and exhaust fans will ensure that existing equipment continues to operate under optimum conditions. This retrofit converts the existing system to a Zero Emissions Engine Processor, ZEEP.

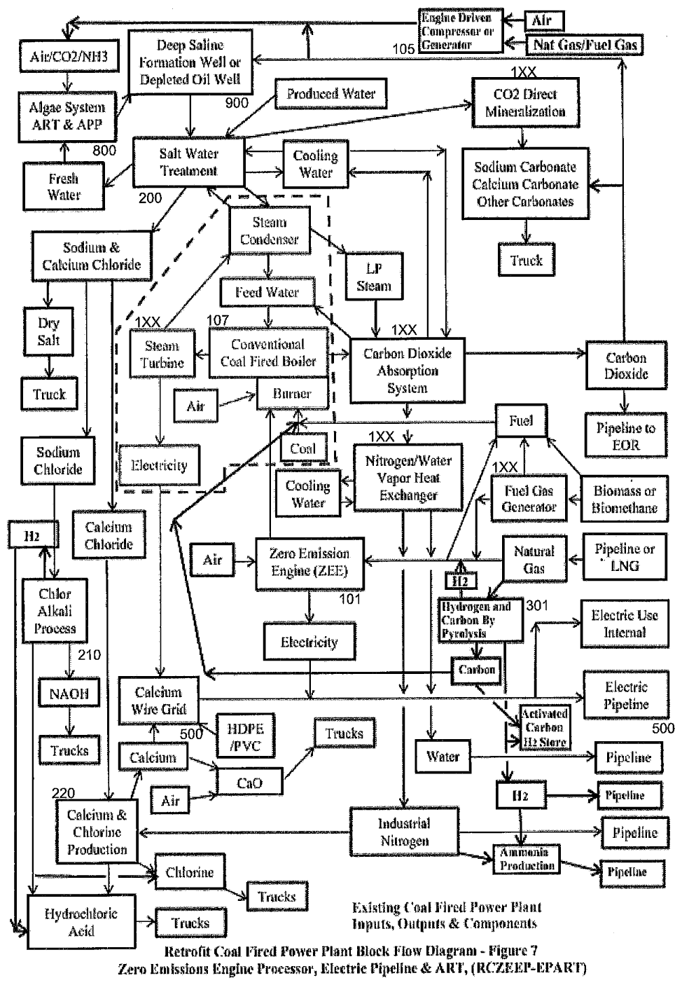

The Block Flow Diagram in FIG. 7 shows the same interaction of the various components of the Zero Emissions Engine Processor, Electrical Pipeline and Algae Related Technologies, (ZEEP-EPART) shown in FIG. 1 with the system retrofitting an existing Coal Fired Power Plant. The dotted line encompasses the existing equipment shown in red text. Additional components such as intake and exhaust fans will ensure that existing equipment continues to operate under optimum conditions. This retrofit converts the existing system to a Zero Emissions Engine Processor, ZEEP.

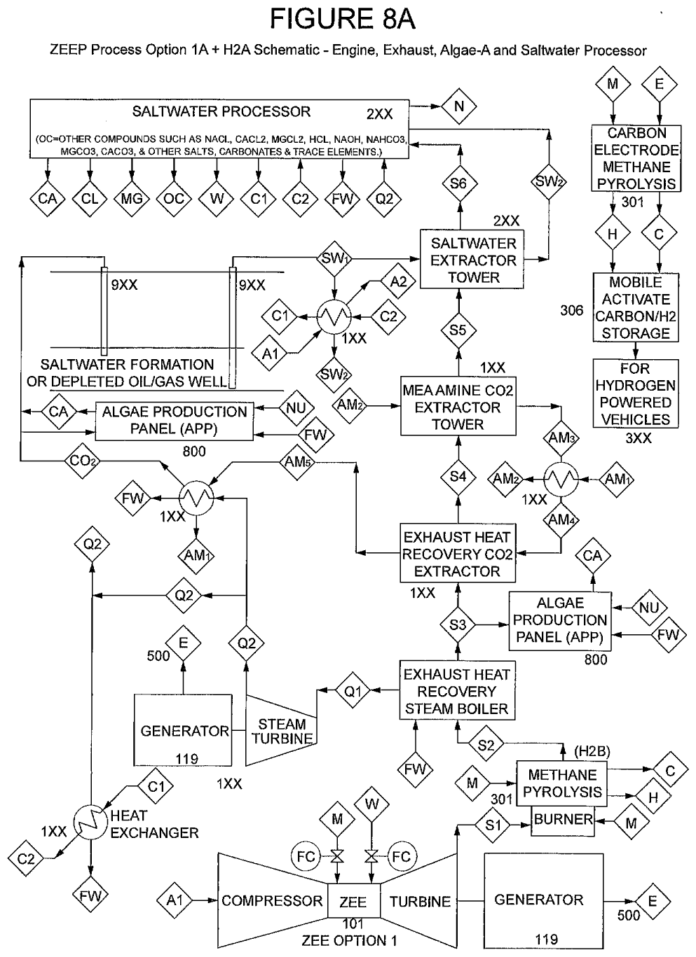

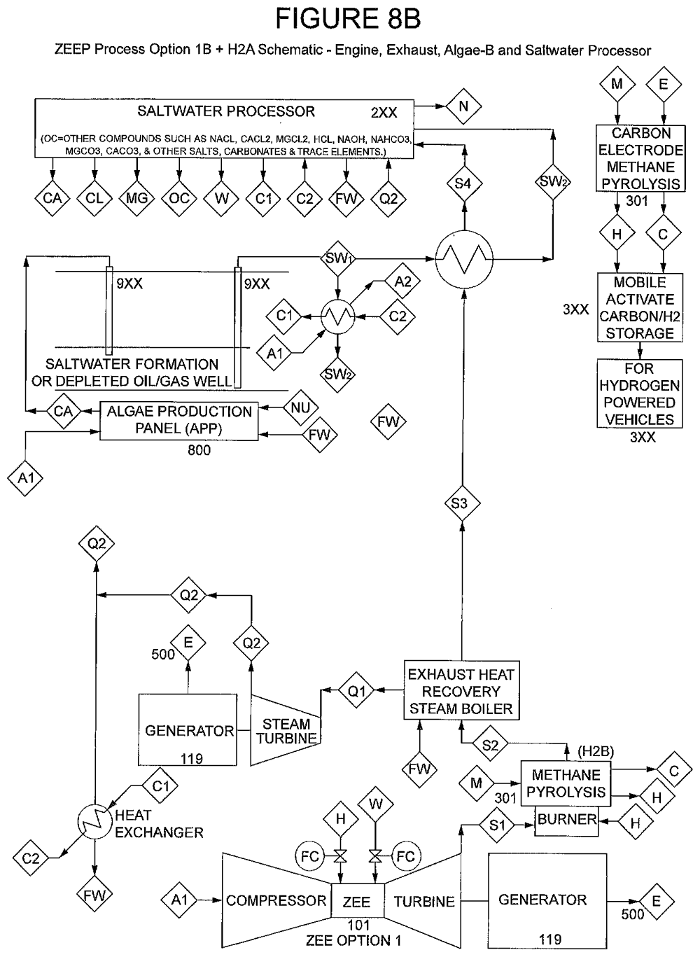

FIG. 8a is a detailed schematic depiction of the Zero Emissions Engine Processor, ZEEP, described in the block flow diagram given in FIG. 1. The schematic includes the Zero Emissions Engine, ZEE, Heat Recovery Steam Boiler, Steam Turbine, Steam Condenser, Carbon Dioxide Absorption System, Saltwater Processor, Cooling Water C1 & C2 and Deep Saline Wells. Air A1, enters the Compressor and is compressed to a set pressure then enters the ZEE, where Gaseous Fuel M is injected and ignited by spark. Water W is injected to control temperature. Combustion energy is converted into mechanical energy by the Turbine, which drives a Generator. Turbine exhaust S1 Enters the Exhaust Heat Recovery Steam Boiler where feed water FW is boiled to produce high pressure high temperature steam Q1. Additional fuel is added as necessary to consume excess oxygen in the exhaust S1. Steam Q1 enters the Steam Turbine, where it is converted into mechanical energy driving an additional Generator. Low Pressure Steam Q2 exits the steam turbine and is piped to a condenser where it is converted in back to feedwater using cooling water C1, to the Amine Regenerator where it is used to heat Amine AM5, releasing CO2 or to the Saltwater Processor where it is used to heat and boil saltwater and condensing back to Feed Water FW. ZEE exhaust S2 leaves the Exhaust Heat Recovery Steam Boiler and enters the Concentrated Saltwater Spray Dryer & Exhaust Cooler where it vaporizes the concentrated saltwater leaving Dry Salts and Compounds. Cooled exhaust S3 then enters The Exhaust Heat Recovery CO2 Extractor where it preheats Amine AM4 to AM5 prior to it entering the Amine Regenerator. Further cooled exhaust S4 then enters the Amine CO2 Extractor Tower where the CO2 is stripped from the exhaust by Amine A2. Further cooled exhaust S5 then enters the Saltwater CO2 Extractor Tower/Cooler where the remaining trace of CO2 is removed, and exhaust is cooled to S6 and Saltwater SW1 from the Formation is heated to SW2. Further cooled exhaust S6 then enters the Saltwater Processor along with Saltwater SW2, Steam Q2 and Cooling Water C. The Saltwater Processor takes Saltwater SW2 and produces Calcium, CA, Chlorine, CL, Magnesium, MG, Concentrated Saltwater SW3 and Other Compounds. Carbon Dioxide, CO2, is removed from the ZEE exhaust using Lean Amine AM1, which is cooled by Amine stream AM3 in the Amine Heat Exchanger, then enters the Amine CO2 Extractor Tower and is converted to Rich Amine AM3 then enters the Amine Heat Exchanger where it is heated to AM4 then to the Amine Exhaust Heat Recovery unit, then leaves as AM5 then enters the Amine Regenerator where the Rich Amine is converted to Lean Amine AM1. The CO2 is directed through a pipeline to a Saltwater Formation or other destination.

FIG. 8b shows the same interaction of the various components of the Zero Emissions Engine Processor, ZEEP, described in the detailed schematic given in FIG. 8a with the addition of Hydrogen production using methane pyrolysis to produce hydrogen and carbon with no CO2 being produced. The carbon will be used to make activated carbon, which will be packed into metal containers and infused with hydrogen. These containers will then be used to supply hydrogen to mobile vehicles. It is estimated that 5 pounds of hydrogen can be absorbed by 50 pounds of activated carbon. The hydrogen will be released by regulating the container or temperature or pressure.

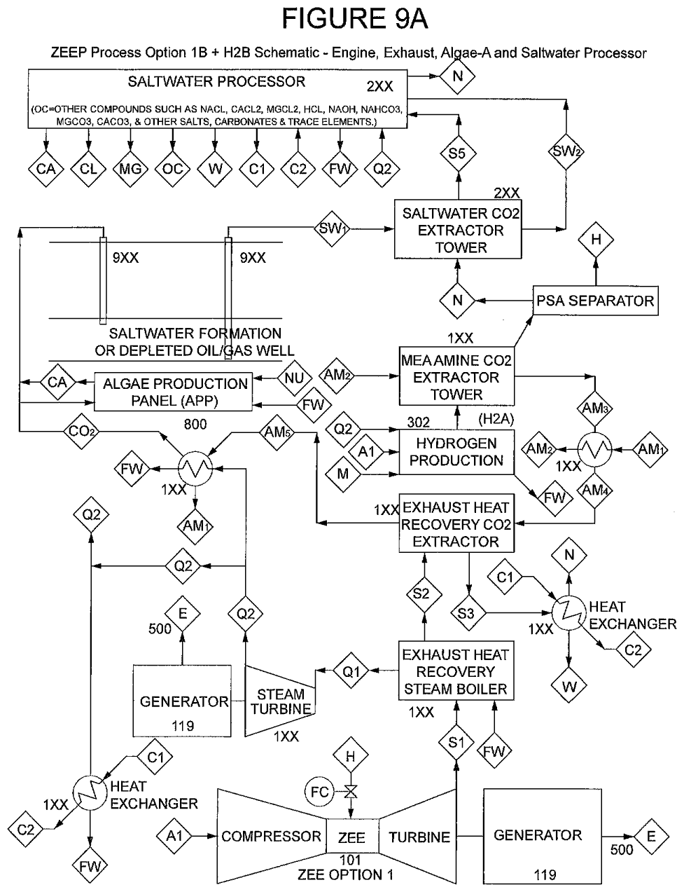

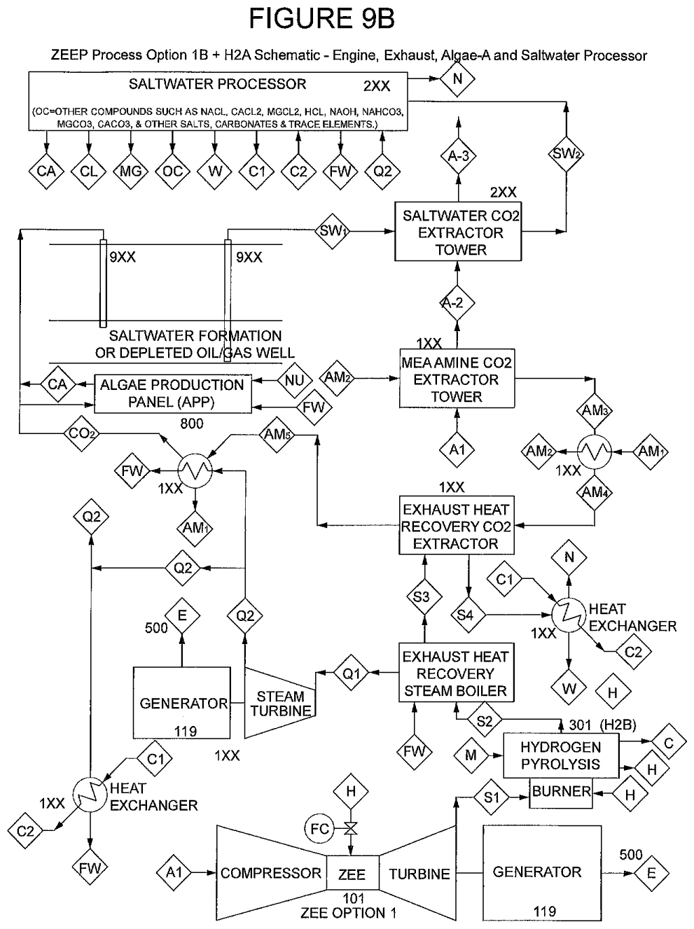

FIG. 9a shows the same interaction of the various components of the Zero Emissions Engine Processor, ZEEP, described in the detailed schematic given in FIG. 8a with the fuel being Hydrogen and CO2 being captured from the exhaust of the water shift Hydrogen Production Unit.

FIG. 9b shows the same interaction of the various components of the Zero Emissions Engine Processor, ZEEP, described in the detailed schematic given in FIG. 8b with the fuel being Hydrogen.

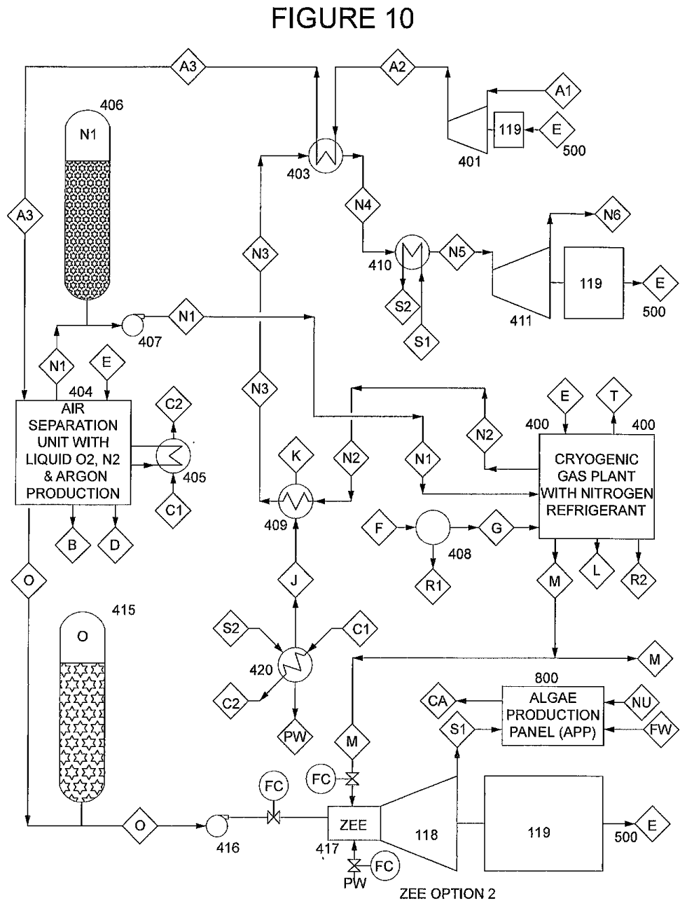

FIG. 10 is a detailed schematic depiction of the Zero Emissions Engine Processor, ZEEP, described in the block flow diagram given in FIG. 3, with the focus on the interaction of Air Separation Unit, Cryogenic Gas Plant and Zero Emissions Engine. Other components in Block Diagram 3 are given in detailed schematics given in FIGS. 8 and 9.

Air, (A1), enters compressor, (1), which is driven by electric motor (2) and is brought to sufficient pressure and is then cooled in heat exchanger (3) by nitrogen vapor N3. The pressurized cool air then enters the air separation unit (4), where liquid nitrogen (N1), liquid oxygen (O), liquid argon (B) and trace gases (D) are produced. Cooling Water (C1) is supplied to heat exchanger (5) to allow for heat transfer, producing warm water (C2). Liquid nitrogen (N1) leaves the air separation unit (4) and enters tank (6) for storage or pump (7) for distribution to other processes. In one process, pressurized liquid nitrogen (N1) enters Gas Plant (9), where it is vaporized by cooling various process streams inside the cryogenic gas plant. The cold vaporized nitrogen N2 leaves the gas plant (9) then enters heat exchanger (21) where it produces heated vaporized nitrogen N3 by cooling a mixture of CO2 and water vapor, condensing water, prior to entering heat exchanger (3) where it produces heated vaporized Nitrogen N4 by cooling pressurized Air A2 prior to entering heat exchanger (10) where it is further heated by exhaust steam S1 producing hot vaporized nitrogen N5. The hot pressurized nitrogen vapor N5 is then expanded in turbine (11), producing electricity E in generator (12). The warm low pressure nitrogen vapor N6 is then distributed to other processes.

Liquid oxygen (O) leaves the air separation unit (4) and enters tank (15) for storage or pump (16) for distribution to the Zero Emissions Engine, ZEE, burner (17) where it combines with dry natural gas or Methane M combining to produce a mixture of high-pressure steam and carbon dioxide vapor S1. This Vapor S1 drives turbine (18) and generator (19), producing electricity E and is then routed to heat exchanger (10), where it is used to heat nitrogen N4 to higher temperature N5. The cooler steam and carbon dioxide vapor S2 is routed to the heat exchanger 13, where it is used in flash water off Saltwater Stream SW3 leaving dry salts. The still cooler steam and carbon dioxide vapor S3 is routed to the heat exchanger 20, where cooling water C1 condenses water W out of the vapor S3. Nearly pure CO2 J is directed to heat exchanger 21, where it is further cooled by nitrogen N2 to form pure carbon dioxide K. Water W may contain trace carbon dioxide, so post treatment may be required before it is exported or fed back to the ZEE burner 17, The cryogenic gas plant (9), with the added nitrogen refrigerant, processes the wet natural gas G from pipeline gas F via Slug Catcher (8) into dry natural gas of Methane M, natural gas liquids R2, liquid natural gas L and trace gases (T). The excess electricity produced from the Zero Emissions Engine Processor, ZEEP, is free from environmental impacts due to nitrogen oxide, NOX, and carbon dioxide, CO2, emissions. The processor application is not limited to the oil field but can be applied to any location through the addition of minor modifications.

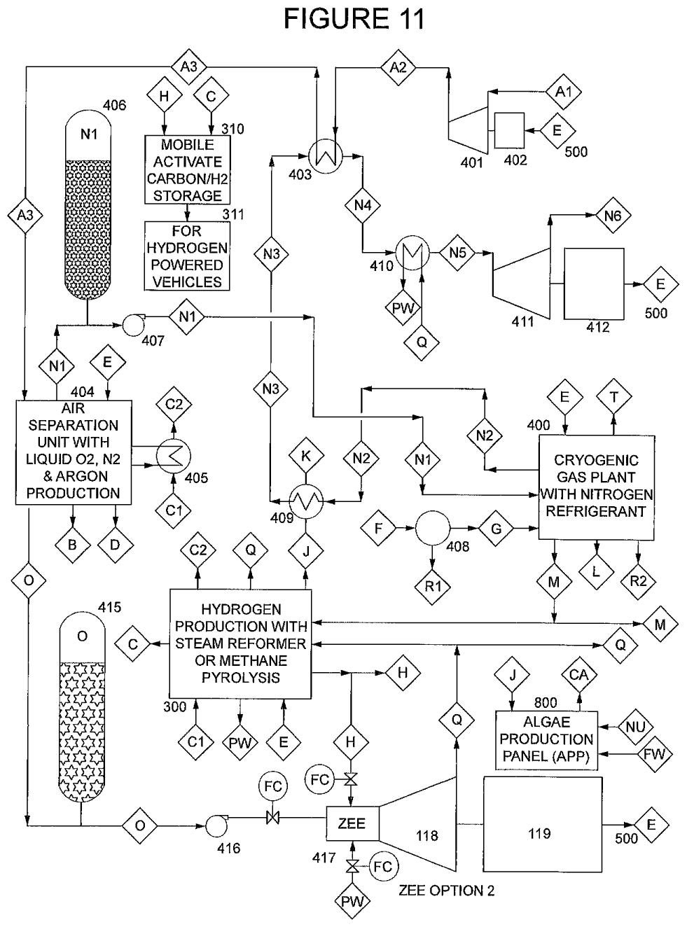

FIG. 11 is a modification FIG. 10 where hydrogen from the hydrogen production unit (14) and pure water P, are used to produce high pressure high temperature steam Q in the Zero Emissions Engine 17, which drives a turbine 18 and generator 19, producing electricity E. Low pressure Low temperature steam Q1 leaves turbine 18 and is then distributed to other processes including the hydrogen production unit (14), where it is used for the production of hydrogen H from dry natural gas or methane M along with carbon dioxide J, water W and steam Q2. The resulting hydrogen (H) is sent to the ZEE burner 17 and distributed to other processes. All other processes are the same as in FIG. 10.

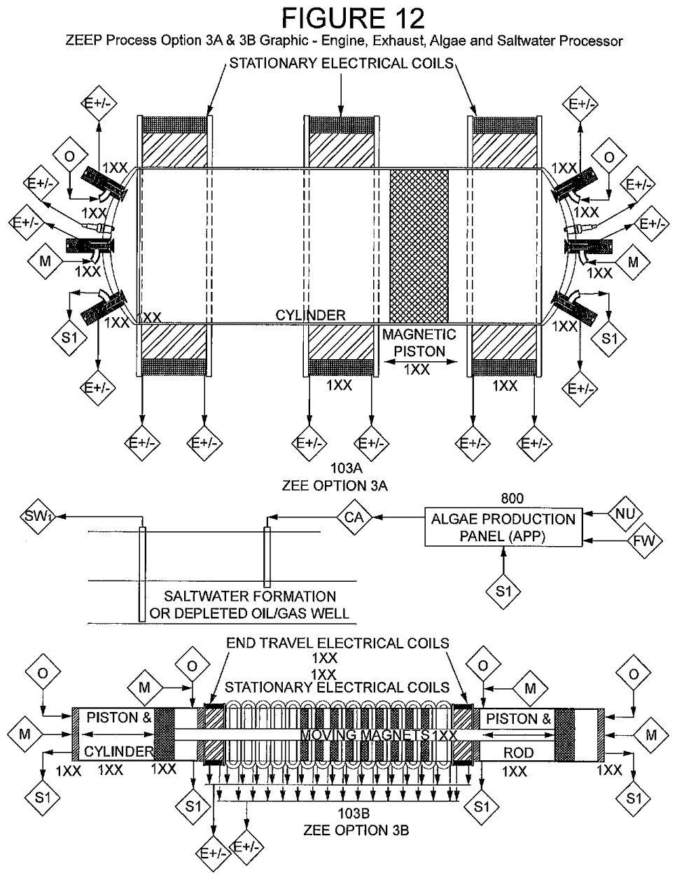

FIG. 12 contains graphic depictions of two alternate versions of the zero emissions engine, ZEE depicted in FIG. 10 with a unique Zero Emissions Piston Engine concept. In Option 3A, oxygen is combined with natural gas as fuel, the Zero Emissions Piston Engine, ZEPE, produces power by moving a double acting piston from one end of the cylinder to the other. Temperature is controlled by water injection after combustion. The piston contains a permanent magnet and coils are located on the outside of the thin wall nonferrous cylinder. The cycle begins when the piston is at one end of the cylinder and oxygen and fuel are injected, causing an explosion that moves the piston to the opposite end. As the magnetic field of the piston passes the coils, an electrical current is produced in the coils, which is processed through electronics and other devices to produce usable electrical power. As the piston moves to the other end of travel, exhaust gases ahead of the piston flow through an open valve to further processing. As the piston approaches the end of travel, the exhaust valve closes as the opposite end exhaust valve opens and oxygen and fuel are injected causing the reversing of the piston direction and the process is repeated. The exhaust products of combustion including CO2 and H2O are directed to a water condenser and finally the underground formation where the CO2 is sequestered by either EOR or Deep Saline Formation Injection. The equalization pressure is determined by the quantity of oxygen and natural gas and the desired or formation pressure. The fuel, oxygen, formation pressure, piston diameter, stroke and speed will be optimized to produce the maximum amount of electrical power, with efficiency ratings ranging from 30%-60%. Intermediate coils can be added as necessary, to maximize electrical production for the cylinder and piston lengths. The piston could be kept centered in the cylinder using electromagnetic force, allowing for close tolerances and non-contact surfaces. The low cost and few moving parts make this Zero Emissions Piston Engine, ZEPE, superior over conventional engine technologies. The ZEPE can operate on Air and Natural Gas or Hydrogen by operation in a four-stroke configuration. Intake, Compression, Power and Exhaust can be performed with the assistance of the coils driving the piston during some of the strokes.

Another design shown as option 3B uses two cylinders with two pistons connected by a shaft. This allows for the magnetic coils to be closer to the permanent magnets and away from the combustion temperatures. The same operational principles apply, with additional options for electrical production. The ZEPE can operate on Air and Natural Gas or Hydrogen when used in a four-stroke configuration. Intake, Compression, Power and Exhaust can be performed without the assistance of the coils because a power stroke occurs during each movement of the piston.

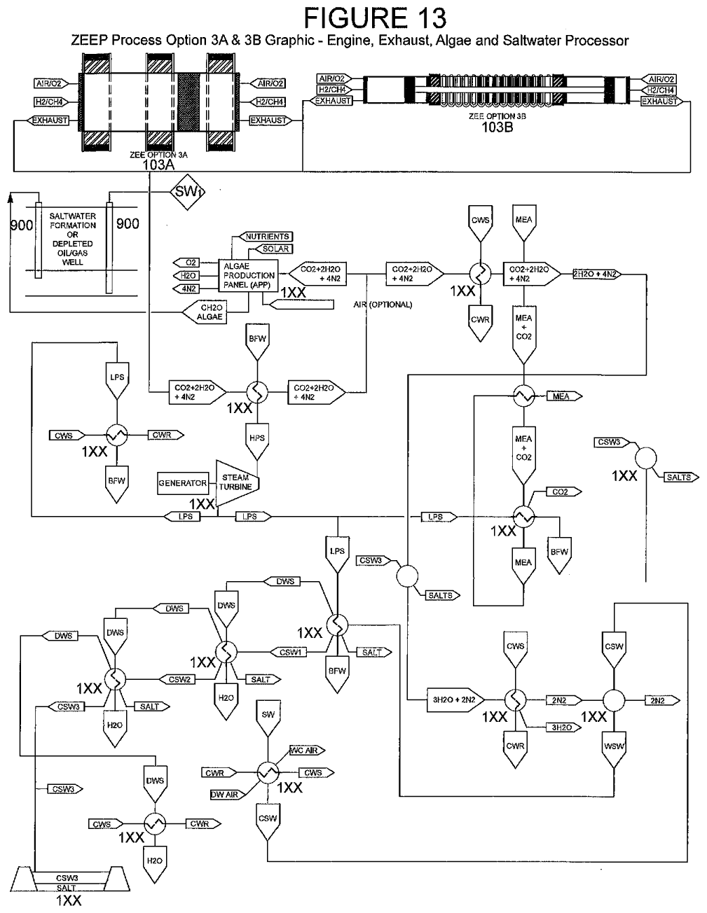

FIG. 13 contains schematic depictions of two alternate versions of the zero emissions piston engine, ZEPE depicted in FIG. 12 with their interaction with other systems including a heat recovery steam boiler, carbon dioxide recovery system and a saltwater processor. Fuel and Air or Oxygen enter the engine and Exhaust exits and goes to heat Recovery Boiler 13A where it boils Boiler Feed Water BFW to produce High Pressure Steam HPS, which Drives the Steam Turbine and Generator. The exhaust then passes through a Flash Dryer 13B where Concentrated Saltwater CSW3 is boiled off leaving dry Salts. The cooler Exhaust then Enters cooler 13C, where it is further cooled before entering the Amine Tower 13D. Lean Amine MEA enters the top of 13D and absorbs CO2 and exits the bottom. The clean Exhaust exits the top of 13D and enters Condenser 13E where water is removed by Cooling Water Supply CWS leaving as Cooling Water Return CWR. Remaining warm Nitrogen Exhaust then passes through final direct contact Cooler 13F, where it warms Concentrated Saltwater CSW to Warm Concentrated Saltwater WSW before exiting 13F. WSW exits 13F and enters the first of three evaporator effects 13G, where Low Pressure Steam LPS is used to boil off saltwater, producing Concentrated Saltwater CSW1, Distilled Water Steam DWS and Salt slurry. LPS condenses into Boiler Feed Water BFW. Stream CSW1 proceeds to the second effect evaporator, 13H, where DWS from Stage 1 is used to boil off saltwater producing CSW2, DWS and Salt slurry. Stage 1 DWS condenses into Water H2O. Stream CSW2 proceeds to the third effect evaporator, 13J, where Stage 2 DWS is used to boil off saltwater producing CSW3, DWS and Salt slurry. Stage 2 DWS condenses into H2O. DWS from 13J enters Condenser 13K and is condensed into H2O by CWS leaving as CWR. CSW3 leaves 13J and goes to either Evaporating Pond or Flash Dryer 13B. Saltwater from Deep Saline Well or Produced Water SW enters Evaporative Cooler 13L where Air is used to absorb water, cooling CWR down to CWS and making Concentrated Saltwater CSW, which then enters 13F. Amine and CO2 leaves 13D and cools MEA in Heat Exchanger 13M then proceeds to Amine Regenerator 13N where LPS heats the solution and drives off CO2, leaving Warm MEA. Warm MEA is cooled by 13M before entering 13D. LPS from the Steam Turbine enters 13G, 13N or Condenser 13P, where CWS is used to condense LPS to BFW, leaving as CWR.

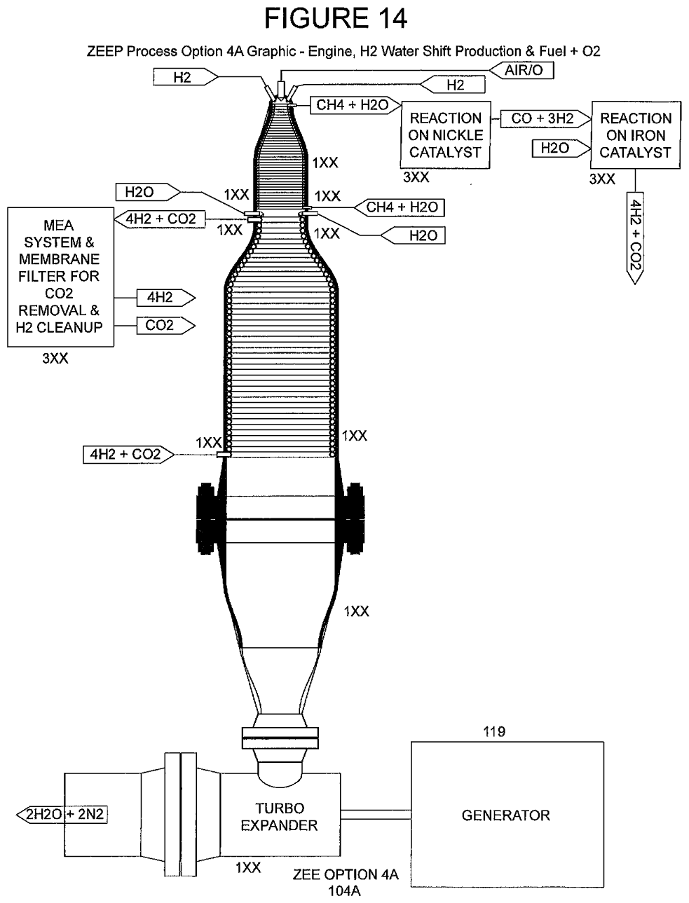

FIG. 14 contains graphic depictions of an alternate versions of the zero emissions engine, ZEE depicted in FIGS. 9 & 11. The design eliminates the compressor by supplying pressurized air or oxygen or liquid oxygen O and combines it with equally pressurized hydrogen H. Liquid water is injected after complete combustion to lower the temperature to match the limits of downstream components. The unit can be fabricated from rolled steel plate or pipe and fittings. The inside surface of the vessel is lined with ceramic insulation tiles or process tubing to protect the walls from high temperature and provide process heat for methane steam reforming or other process. Air or Oxygen O and Hydrogen H2 enter the Combustion Chamber at high pressure and are ignited for continuous combustion. Products of combustion expand and heat the Ultra High Temperature Coil. Water is injected after complete combustion has occurred just prior to the expansion section and is vaporized at high pressure, reducing the temperature of the exhaust. A high-Pressure Coil is provided for process connection or heat recovery from Steam Reformer Process. The Steam Chamber allows for complete water vaporization and stable flow through optional heat exchanger that can be inserted between flanges. The exhaust flow continues through a reducer section to increase velocity before it enters the Turbo Expander, where energy is extracted to drive a generator or other device. Low pressure exhaust is directed through the Exhaust Section to other processes.

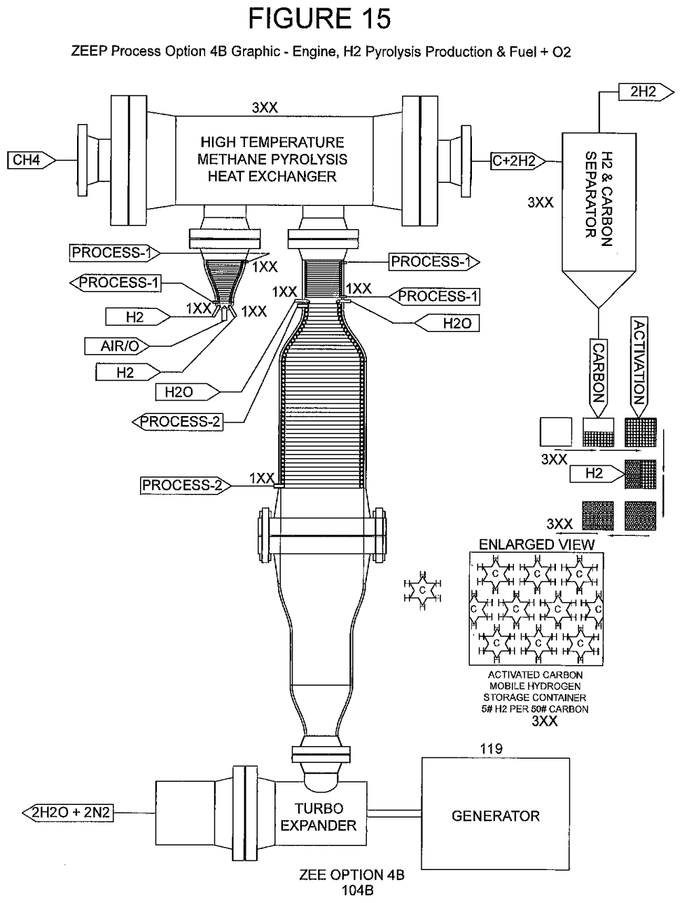

FIG. 15 contains graphic depictions of an alternate versions of the zero emissions engine, ZEE depicted in FIG. 14A with the addition of a Ultra High Temperature Heat Exchanger that allows more surface area than the internal coil and uses the 2000 to 3000 degree f combustion gasses to heat natural gas to Pyrolysis temperatures converting it to Carbon and Hydrogen. A catalyst coated assembly can be inserted in the tubes to promote the reaction and increase heat transfer. The Hydrogen produced can be split between the engine requirements and product export. The internal tube lining could be used for other processes. All other functions match the unit described in FIG. 14A. Hydrogen production is accomplished using methane pyrolysis to produce hydrogen and carbon with no CO2 being produced. The carbon can be used to make activated carbon, which can be packed into metal containers and infused with hydrogen. These containers will then be used to supply hydrogen to mobile vehicles. It is estimated that 5 pounds of hydrogen can be absorbed by 50 pounds of activated carbon. The hydrogen will be released by regulating the container or temperature or pressure.

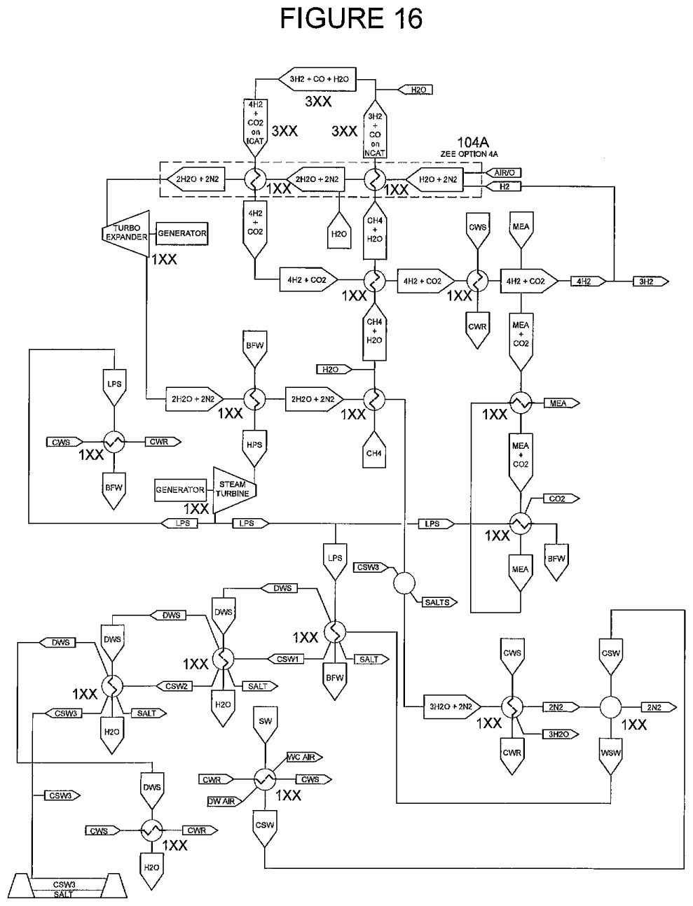

FIG. 16 contains schematic depictions of zero emissions engine, ZEE depicted in FIG. 14 and its interaction with other systems including a heat recovery steam boiler, carbon dioxide recovery system and saltwater processor. In this case, Hydrogen fuel and Air enter the engine and is ignited by a spark. Ultra-High Temperature Combustion Products expand and heat the internal coil 15A containing a mixture of water and methane as part of a methane Steam Reformer Process. The mixture then enters a reactor with Nickle Catalyst 15B where Carbon Monoxide and Hydrogen are formed. Water is added to the stream and the stream enters the second reactor with Iron Catalyst 15C, where Carbon Dioxide and additional Hydrogen is produced, then to coil 15D, where heat is recovered from the stream. The reheated nitrogen and water vapor then exits the expansion section through an optional heat exchanger to a Turbo Expander, where energy is extracted to drive an electrical generator or other device. The cooler exhaust then enters an optional Expansion Turbine where additional energy is extracted to drive an additional electrical generator or other device. The still cooler exhaust then enters a Heat Recovery Steam Boiler 15E where it boils Boiler Feed Water BFW to produce High Pressure Steam HPS, which Drives the Steam Turbine and Generator. The exhaust then passes through a Methane Preheater 15F then Flash Dryer 13G where Concentrated Saltwater CSW3 is boiled off leaving dry Salts. The cooler Exhaust then enters Condenser/Cooler 15H, where the nitrogen is cooled and the water vapor is condensed by Cooling Water Supply CWS leaving as Cooling Water Return, CWR. The remaining warm Nitrogen is cooled to wet bulb temperature by passing through an optional Spray Column 15J, where Nitric Oxide and other pollutants are removed by the Concentrated Saltwater spray, CSW from Evaporative Cooler 15K. The Nitrogen is vented to atmosphere or diverted to other processes, and the concentrated saltwater CSW is directed to the First Stage Evaporator, 15L. Saltwater from a Deep Saline Formation or other source enters Evaporative Cooler 15K and is partially evaporated by Dry Warm, DW Air and producing Wet Cool, WC Air and Cool Concentrated Saltwater CSW. The CSW cools the Cooling Water Return CWR through an internal heat exchanger in 15K producing Cooling Water Supply, CWS. CSW leaves 15K and enters Spray Column 15J or First Stage Evaporator, 15L if 15J is not installed. CSW is partially evaporated in the First Stage Evaporator, 15L by Low Pressure Steam, LPS from the Steam Turbine, producing Boiler Feed Water, BFW and Higher Concentrated Saltwater CSW1, Distilled Water Steam DWS and Crystalized Salts. Stream CSW1 and DWS proceed to the Second Stage Evaporator, 15M, where DWS from Stage 1 is used to boil off saltwater producing CSW2, DWS and Crystalized Salts. Stage 1 DWS condenses into Water H2O. Streams CSW2 and DWS proceed to the Third Stage Evaporator, 15N, where Stage 2 DWS is used to boil off saltwater producing CSW3, DWS and Crystalized Salts. Stage 2 DWS condenses into H2O. DWS from 15N enters Condenser 15P and is condensed into H2O by CWS leaving as CWR. CSW3 leaves 15N and goes to either Evaporation Pond or Flash Dryer 15G. Methane enters Preheater 15F and is heated prior to water addition in the form of High Temperature Steam. The methane and steam then enter heat Exchanger 15Q where it is further heated by the CO and Hydrogen stream leaving the Internal Heat Exchanger 15D. The heated steam and methane enter the internal heat exchanger 15A and the cooled CO2 and Hydrogen enters the cooler 15R where it is cooled by CWS then enters the Amine Contact Tower 15S, where the CO2 is captured by amine MEA, leaving hydrogen to pass to ZEE and other uses. Lean MEA enters the top of the tower and Rich MEA, with CO2 absorbed, exits the bottom, then enters heat exchanger 15T where it cools the regenerated Lean MEA before entering the Amine Regenerator 15U where low pressure steam LPS heats the Rich MEA releasing the CO2 then condensing into BFW. The CO2 is directed to further processing and then sequestration. The lean MEA is then cooled by 15U, then sent back to the top of 15S. LPS from the Steam Turbine enters 15U, 15L or Condenser 15R, where CWS is used to condense LPS to BFW, leaving as CWR.

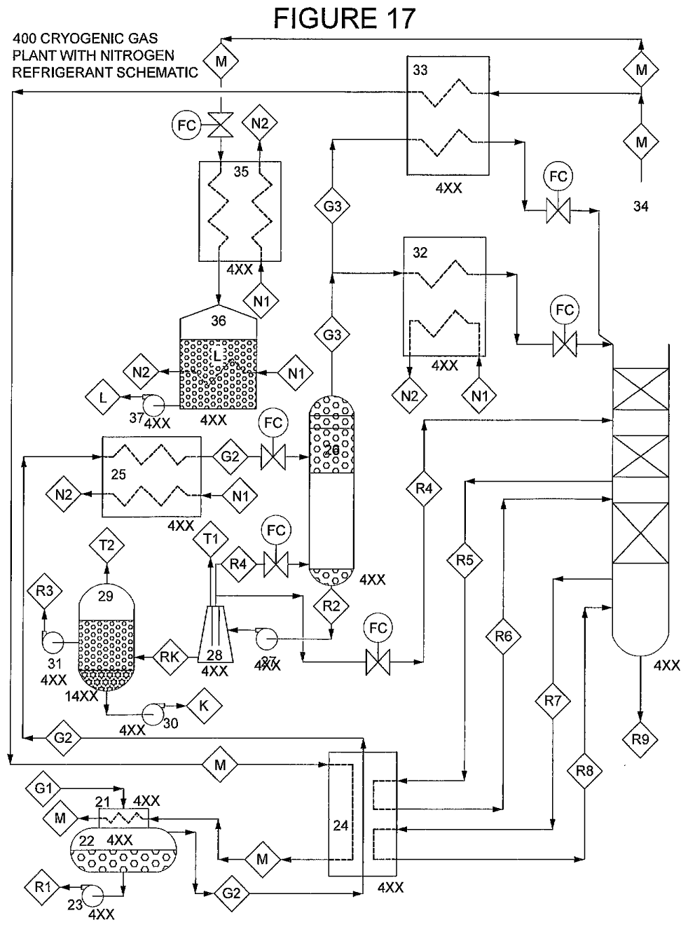

FIG. 17 is an enlargement of the gas processing plant 9 in FIGS. 10 & 11, with emphasis on the liquid nitrogen being used as a refrigerant. Wet gas G1 enters heat exchanger 21, where it is cooled by exiting Methane M condensing out NGLs and Water. The stream then enters vessel 22, where the liquids R1 are separated from the vapor G2. Liquids R1 are removed by pump 23 to storage or transport, and the wet gas G2 enters heat exchanger 24, where it is cooled by exiting Methane M and mixed gases and liquids from the Demethanizer 34. Wet gas G2 then enters heat exchanger 22 where it is further cooled by liquid nitrogen N1, reducing the temperature to that of near carbon dioxide freezing point. Wet gas G2 then enters vessel 23, where the vapor G3 is separated from liquids and solids. The liquid NGLs, frozen CO2 and H2O then enter separator 28, via pump 27, where the ice is separated from the NGLs using centrifugal force. The ice and some NGLs, RK, leave separator 28 and enter vessel 29, where the ice is melted using electric heating elements E, and separated into Liquid CO2 and Water K and NGLs R3. Pump 30 conveys Liquid CO2 and Water K to storage and Pump 31 conveys NGLs R3 to storage or pipeline. Trace vapors T1 and T2 are captured from separator 28 and vessel 29 and conveyed to post processing elsewhere. NGLs R4 from separator 28 are sent either back to vessel 26 or to demethanizer 34. The Vapor G3 from vessel 26 is split into two flows, one entering heat exchanger 32 where the temperature is dropped by liquid nitrogen N1 to a predetermined temperature then entering the demethanizer 34. The other stream enters heat exchanger 33, where the temperature is dropped by exiting methane M, to a predetermined temperature before entering the demethanizer 34. The demethanizer 34 strips ethane and heavier NGLs from the methane, producing NGL streams R5, R7 and R9. Streams R5 and R7 are used to cool entering wet gas G2 in Heat Exchanger 24, which acts as a reboiler for the bottom section of the demethanizer 34. Stream R9 leaves the demethanizer 34 and is conveyed to storage or pipeline. Nearly pure methane, M, exits the top of the demethanizer 34, part of it is conveyed to pipeline or compression via heat exchangers 33, 24 and 21. The other portion of nearly pure methane M enters heat exchanger 35, where it is condensed into liquid natural gas L by liquid nitrogen N1. The liquid natural gas L is stored in tank, 36, and loaded into transport truck by pump 37 for use as transportation or stationary diesel engine fuel as alternative to diesel fuel. All vaporized nitrogen N2 is collected and sent back to the ZEE processor for energy recovery and post processing uses.

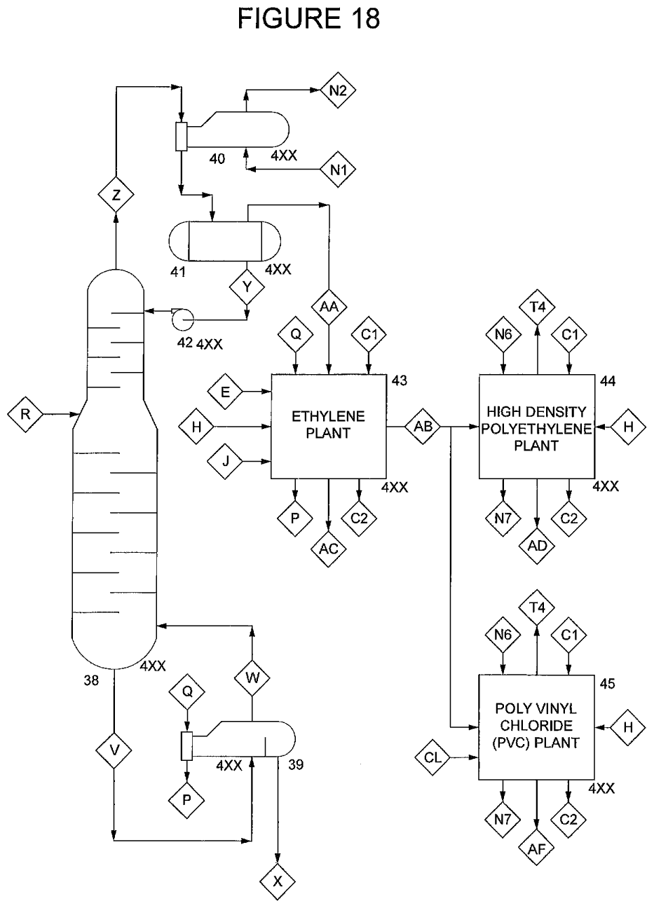

FIG. 18 is an addition to the gas processing plant 9 in FIGS. 10 & 11, with emphasis on the separation of ethane from the natural gas liquids R. This ethane will be further processed into ethylene, then polyethylene for use in the manufacturing coproducts such as High-Density Polyethylene, HDPE, pipe and sheathing for electrical conductors or combined with chlorine to form chloroethylene for use in the production of polyvinylchloride, PVC, products such as pipe and sheathing for electrical conductors. Natural gas liquids R enter the De-Ethanizer column 38 through a control valve, which reduces the temperature and pressure. Mostly heavier than ethane liquid V exits the bottom and mostly ethane and other Vapors X exit the unit from the top. The number of trays is optimized for ethane separation. Liquid V enters Reheat Boiler 39, which vaporizes the lighter liquids W and sends them back to column 38. The heavier than ethane liquid X leaves the bottom of the reheat boiler 39. The reheat boiler is powered by Steam Q, which condenses to pure water P. The vapors X enter heat exchanger 40 where it is cooled with nitrogen refrigerant from the ZEE processor to condense the heavier than ethane vapors. Propane refrigerant can also be used for this process. The cooled vapor X then enters separator 41, where ethane vapor AA leaves the top and heavier than ethane liquids Y exits the bottom and enters pump 42 before returning to column 38. Ethane AA enters an ethylene plant 43, where various processes convert it to ethylene AB. Other Feeds include Steam Q, Electricity E, Hydrogen H, Carbon Dioxide J and Cooling Water C1. Byproducts Include Pure Water P, Carbon Monoxide AC and Warmed Cooling Water C2. The ethylene AB is then directed to a polyethylene plant 44, there it is converted to polyethylene pellets to be used in the fabrication of high-density polyethylene pipe and sheathing for electrical conductors. Other feeds include Nitrogen N6, Hydrogen H and Cooling Water C1. Byproducts Include Pure Water P, Nitrogen N7, Trace Gases T4 and Warmed Cooling Water C2. Expanded detail of the ethylene plant 43 is given in FIG. 18. Another option is for the ethylene to be directed to a vinyl plant 45, where it is combined with chlorine to form chloroethene for use in the production of polyvinylchloride, PVC, products.

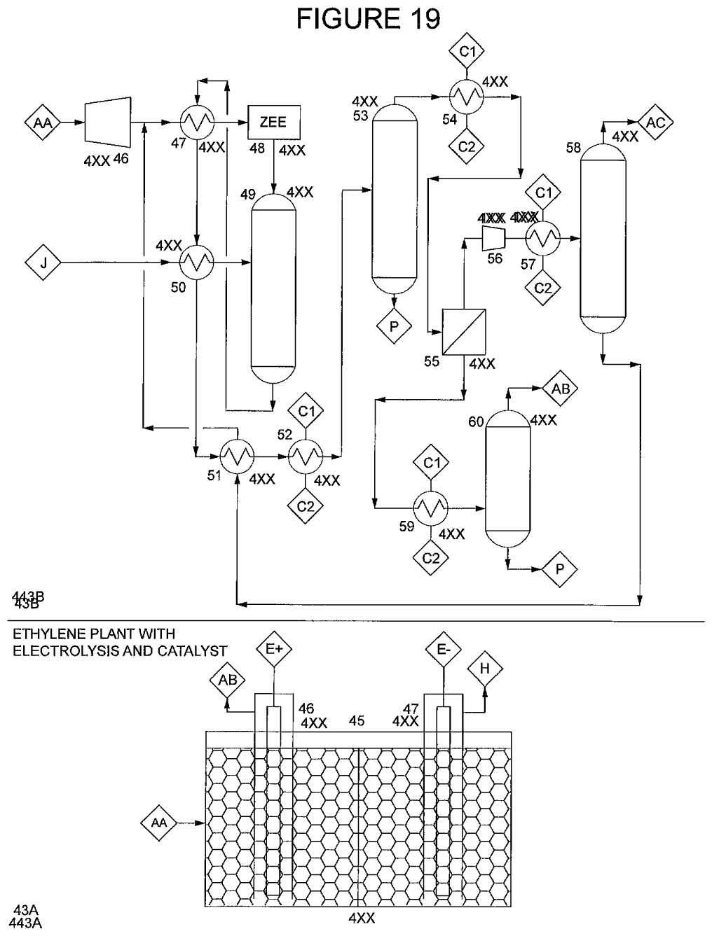

FIG. 19 shows two concepts for the ethylene plant 43 shown in FIG. 17. Ethylene plant 43A uses an electrolysis method of converting ethane AA into ethylene AB. Direct current is supplied to the Electrochemical Cell 45 through the Electro-Catalysts, producing Ethylene AB at the Anode 46 and Hydrogen H at the Cathode 47. Process is like that described in the article: Temperature Electrical Activation of Ethane for Co-production of Chemicals/Fuels and Hydrogen, by Brian Valentine, Technology Manager, U.S. Department of Energy Advanced Manufacturing. Ethylene plant 43B uses Carbon Dioxide J as a soft Catalyst similar to that described in the report Reforming and Oxidative Dehydrogenation of Ethane With CO2 As A Soft Oxidant Over Bimetallic Catalysts, authored By MyatNoeZin Myint, Binhang Yan, Jie Wan, Shen Zhao, and Jingguang G. Chen, Submitted to Journal of Catalysis on November 2016 by the Chemistry Department, Brookhaven National Laboratory, U.S. Department of Energy, USDOE Office of Science (SC), Basic Energy Sciences (BES) (SC-22).

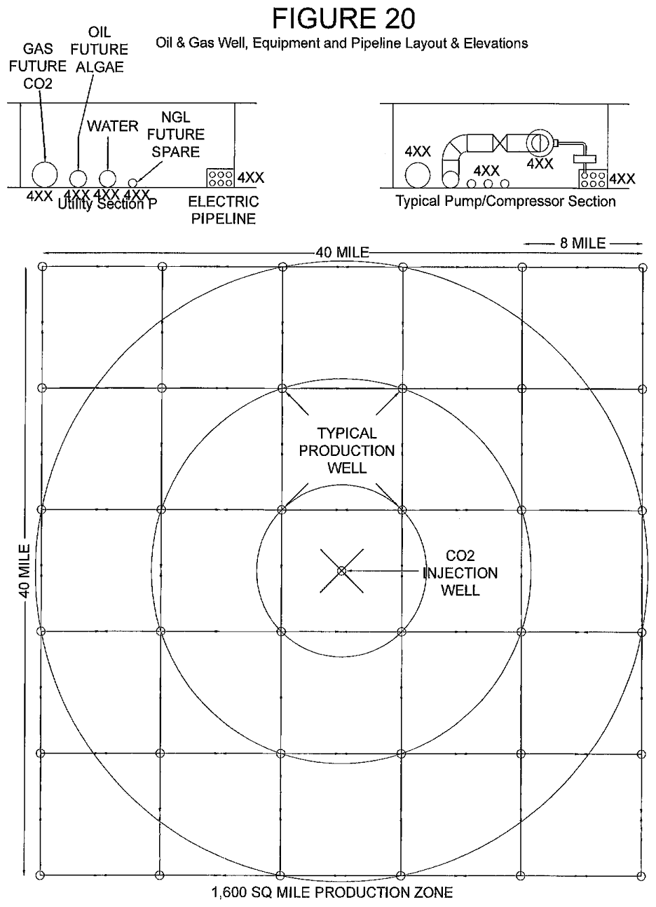

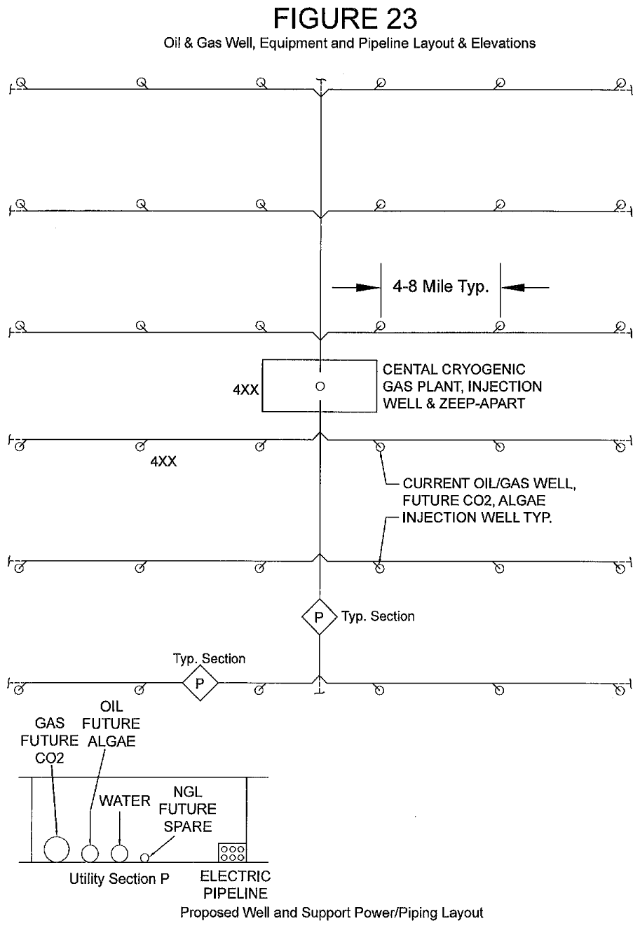

FIG. 20 is a schematic depiction of the embodiment depicted in FIG. 23 with detail of the corridors that move products to and from the well pads. It also describes the well layout of a 1,600 square mile production zone with CO2 Injection in the center pushing oil and natural gas outward moving the injection outward as wells are depleted. The layout can eventually be used to inject algae for CO2 sequestration and eventual renewable oil and gas production. A well-placed injection well would initially provide natural gas for the operation of the electric pipeline and also the gas processing plant. Once surrounding wells are producing natural gas this centrally located injection well will be used to inject CO2 into the formation as an enhanced oil and gas recovery method. Once electrical production is in place temporary underground electrical conductors good power drilling rigs pumps compressors and provide all electrical power for the operations of the oil and gas field. This electrical power will all be emissions free and the waste heat provided by the system will convert produced water to fresh water and recover their valuable Co products from their produced water.

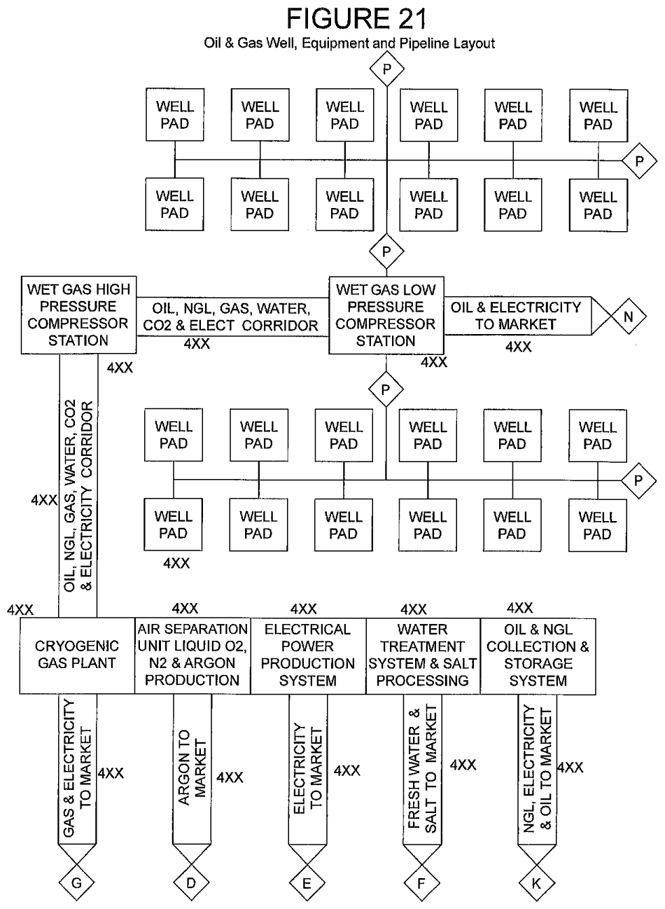

. 21 is a graphic depiction of an expansion of the system depicted in FIG. 21 with the focus on the movement of products, including oil, wet gas, dry gas, produced water, fresh water, proppant, NGLs, CO2, Algae, LNG and electrical power into and out of the oil and gas production zone. When in the oil and gas production area all power requirements can be provided by the electric pipeline. The 50% efficiency of the natural gas fired combined cycle gas turbines far exceed the efficiency of the 30% reciprocating engines currently in use. This savings alone could pay for the system and the countless gallons of diesel fuel used to drill wells and service them will be eliminated. To keep the electricity emissions free the CO2 will be sequestered underground in depleted oil and gas formations or saline formations. Eventually algae produced in surrounding areas would absorb the CO2 and then the analogy would be injected into these depleted oil and gas formations providing future renewable oil and gas.

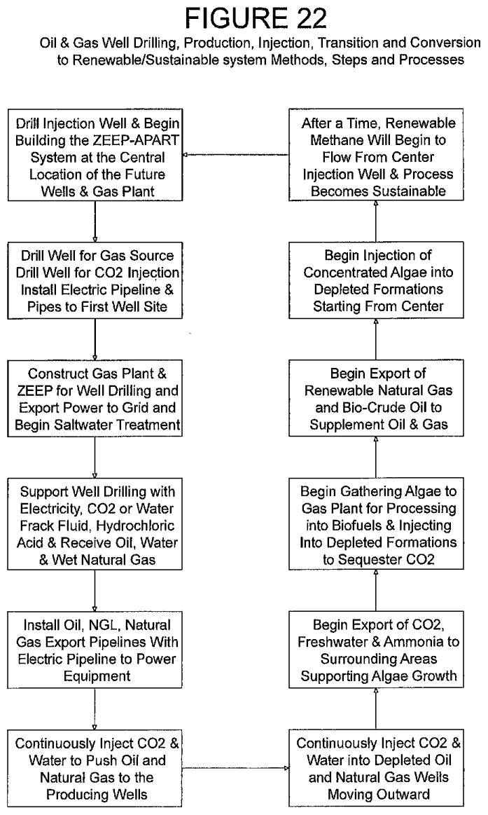

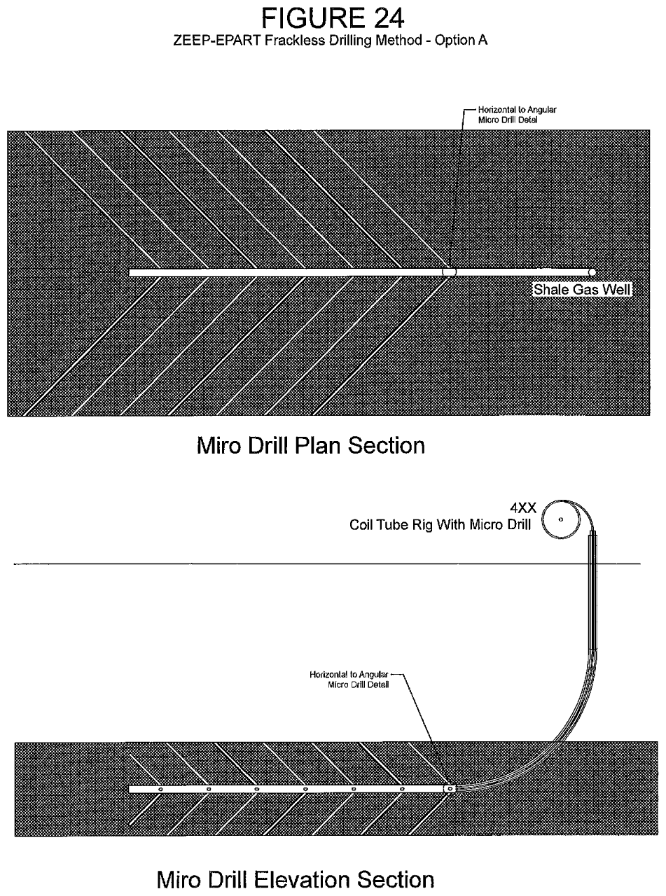

FIG. 22 is a schematic depiction of the embodiment depicted in FIG. 24 with the focus on the step-by-step process from drilling initial well to the transformation of the well to a renewable oil and gas producer. Step one drill injection well and begin building the ZEEP-EPART system at the central location of the future wells and gas plant. Step 2 trail well for gas source drill well for CO2 injection install electric pipeline and pipes to first well site. Step 3 construct gas plant in ZEEP for well drilling and export power to grid and begin saltwater treatment. Step 4 support well drilling with electricity CO2 or water for flat fluid hydrochloric acid and receive oil water and wet natural gas. Step 5 install oil, NGL & natural gas export pipelines with electric pipeline to power equipment. Step 6 continuously inject CO2 and water to push oil and natural gas to the producing wells. Step 7 continuously inject CO2 and water into depleted oil and natural gas wells moving outward. Step 8 begin export of CO2 freshwater and ammonia to surrounding areas supporting algae growth. Step 9 begin gathering algae to gas plant for processing into biofuels and injecting into depleted formations to sequester CO2. Step 10 begin export of renewable natural gas and bio crude oil to supplement oil and gas. Step 11 begin injection of concentrated algae into depleted formations starting from center. Step 12 after a time renewable methane will begin to flow from center injection wells and process becomes sustainable.

FIG. 23 is a schematic depiction of an oil and natural gas production zone that uses the position of the wells to provide enhance hydrocarbon production by injecting CO2 or mixture of CO2 and water to provide continuous washing of the hydrocarbons from the tight or loose formation toward the producing wells. This maximizes the hydrocarbon production and eventually fills the original pore space with CO2 for permanent sequestration. This method could be used to produce tight shale formations without fracking.

FIG. 24 shows a concept for a frackless Coil Tub Rig with Micro-Drilling operation for tight oil and gas formations using horizontal drilling. This concept opens up all kinds of opportunities in countries that do not allow fracking. The use of a micro drill to increase the openings in the formation can provide the same results as fracking. Chance many countries have natural gas in tight shell formations this method would allow them to exploit this resource without violating their commitments to not frack. Using this technology to capture the CO2 also makes the natural gas that they bring to the surface a non-greenhouse gas product as long as they capture the CO2 and put it back into the ground or use it to produce algae or some other method of sequestration. The use of a micro drill may take a little more time to produce but will eventually add more pore space for future algae or CO2 to be sequestered.

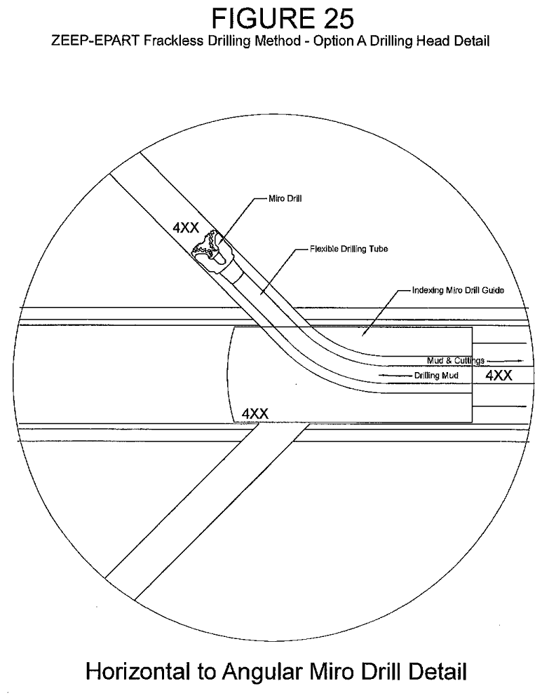

FIG. 25 shows a concept for a Micro Drill Head detail. The micro drill head indexes and allows holes to be drilled in multiple directions. Once the well has been cemented in this head can be moved to multiple locations and the micro drill deployed in multiple directions. There is a flexible pipe connected to the drill head that allows it to make relatively short turns but still functions as a drill pipe with the transfer of cutting fluid pushing the cuttings to the surface.

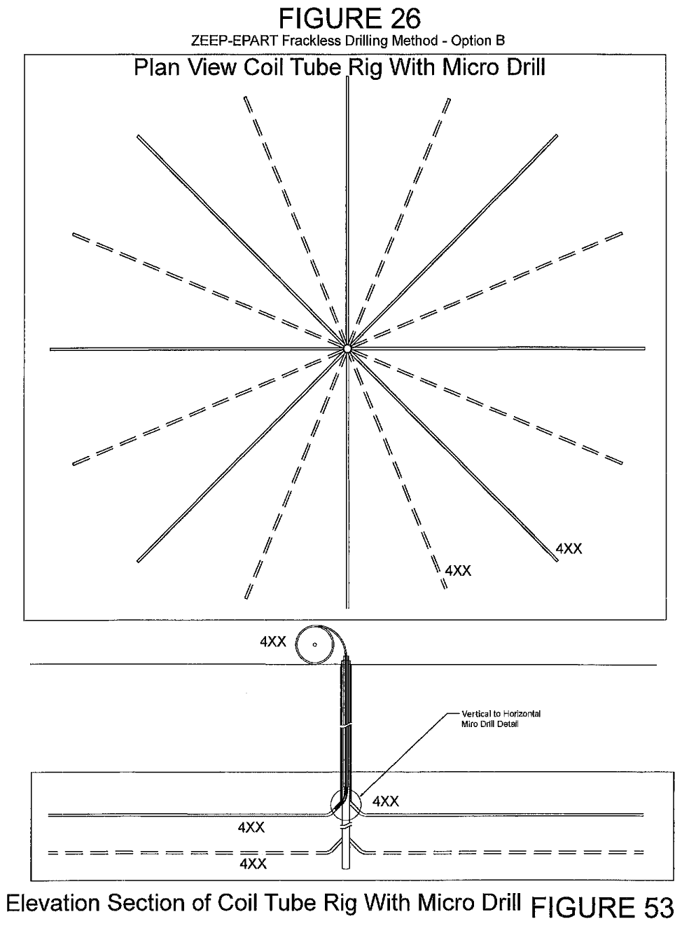

FIG. 26 shows a concept for a frackless Coil Tub Rig with Micro-Drilling operation for tight oil and gas formations using vertical drilling followed by horizontal micro drilling. The same is true for a vertical well once the whale is cemented in the micro drill can drill in very at various levels in the vertical to open up pore space causing oil and natural gas to flow and leaving larger pore space for future sequestration efforts.

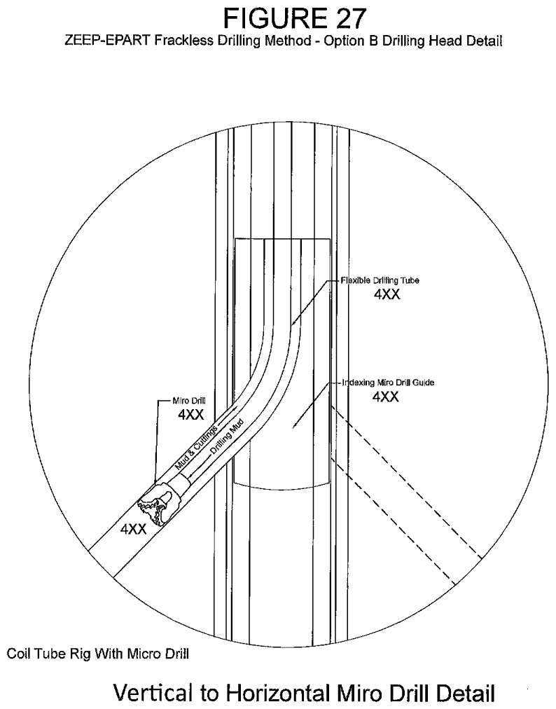

FIG. 27 shows a concept for a Micro Drill Head detail for a vertical well. The micro drill head indexes and allows holes to be drilled in multiple directions. Once the well has been cemented the head can be moved to multiple locations and the micro drill deployed in multiple directions. There is a flexible pipe connected to the drill head that allows it to make relatively short turns but still functions as a drill pipe with the transfer of cutting fluid pushing the cuttings to the surface.

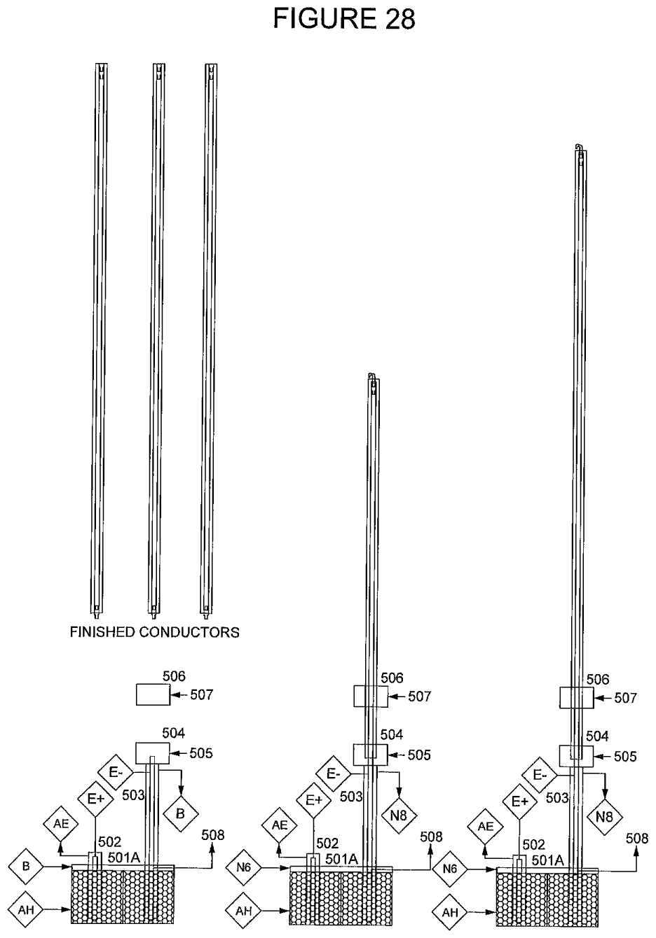

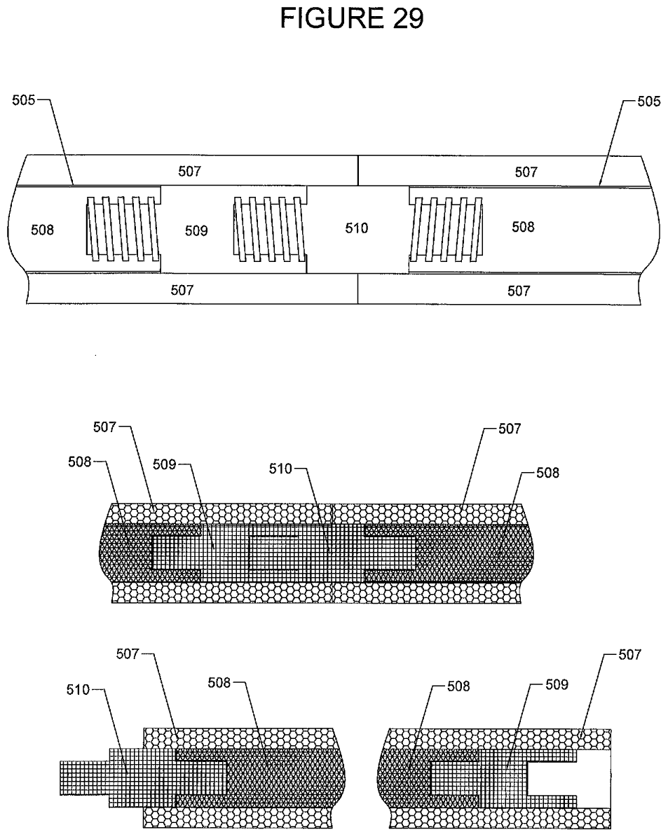

FIG. 28 a schematic depiction of the method to produce pure calcium from calcium chloride generated by the processing of produced water. Calcium is a good conductor of electricity with a lower density and cost per pound than copper or aluminum. Calcium is also a building block for other valuable commodities such as cement, (calcium oxide) and other products. It is believed that the abundance of calcium chloride in saline or produced waters, combined with this process will result in a cleaner more cost-effective process in the production of electrical conductors and cement. Calcium chloride and a mixture of other salts AH are heated to a molten state in an electrochemical cell 501A. Electrical DC voltage is applied across the cell's anode & cathode inducing electrical current to pass through the cell, producing chlorine AE at the anode 502 and calcium metal 508 at the cathode 503. The calcium is deposited on the cathode, producing a circular rod. This process is like that described in U.S. Pat. No. 3,226,311, with added provisions to produce long rods of calcium metal. The cathode 503 is continuously drawn up, out of the cell until the desired length is achieved, shown as 501B & 501C, through an aluminum surface treatment module 504, then on to a sheathing module 506. The calcium rod is passed through an argon B atmosphere as it is withdrawn, until an aluminum protective coating 505 is added on the exterior. The rod is then enclosed in polyethylene, polyvinylchloride or other protective sheathing 507 as an insulator and to eliminate potential decomposition of the calcium caused by oxygen, water or other reactants, creating low-cost electrical conductors. The resultant electrical conductors can be joined together and buried underground providing a means for the distribution of electrical power over long distances. These calcium conductors form the backbone of the proposed electric pipeline. FIG. 29 a schematic depiction of a method to seal the ends of the calcium conductors described in FIG. 28 from corrosion until they can be assembled in the field. It also describes a method of and joining and sealing the calcium conductors together as they are buried underground. Calcium conductor, 508, exit the electrochemical assembly and one end is tapped to receive a threaded assembly 509. This threaded assembly 509 is the female portion of the joining assemble which is made of aluminum or other conductor that will be resistant to corrosion. Once this assembly is attached to the conductor, the joint and conductor is surrounded by the aluminum surfacing 505 and sheathing 507 sealing the calcium from the environment. When the desired length is reached, the conductor is cut and this end is tapped for the male portion of the joining assembly 510, then that end is sealed by the sheathing material 507. The process is then repeated for the next conductor with the female assembly 509 being attached to the next segment.

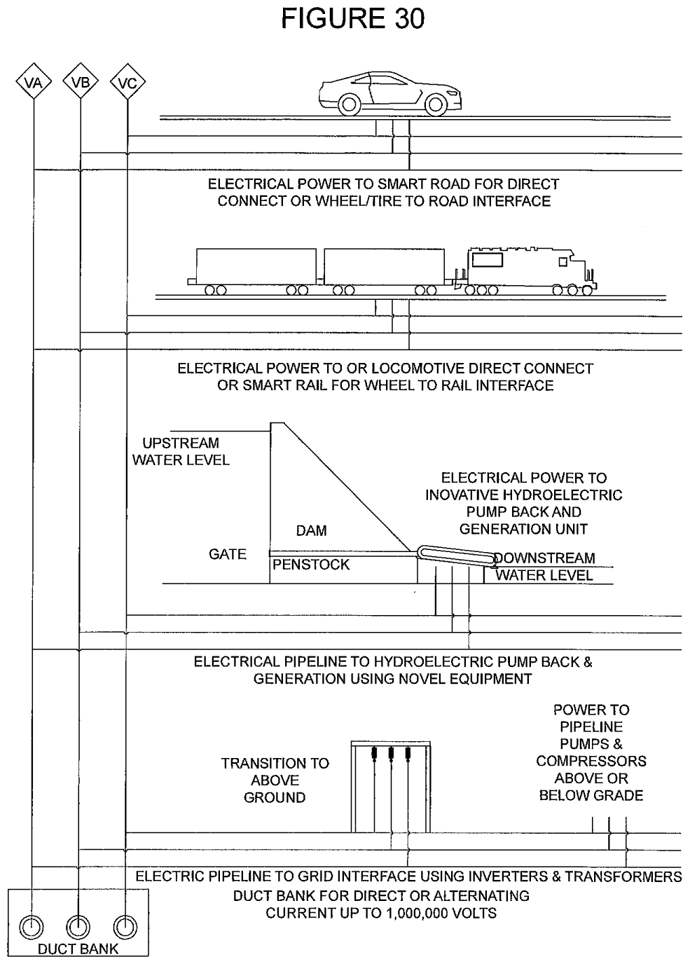

FIG. 30 shows the interaction of the various systems and components of the Electric Pipeline. It is a schematic depiction of an embodiment, the Electric Pipeline, EP, that details the distribution of electricity throughout the grid system and other markets including Electric Highways, Railroads and Waterways using the innovative calcium underground conductor, CUC, or conventional above and below ground conductors. It Introduces a proposed method of transmission of electrical power using two DC high voltage conductors with medium voltage differential between conductors plus a third conductor to act as a neutral. An electronic inverter will create medium AC voltage or pulse current anywhere along the corridor providing power for conventional motors or advanced linear motor driven equipment. This allows power to be transmitted long distances with low voltage drops. The DC Voltage can be as high as 1 million volts. This lowers the amperage which in turn reduces the voltage drop. The two high voltage DC conductors have a voltage difference between them of approximately 13,200 volts. This 13,200 Volt differential operates the DC to AC inverters creating three phase AC voltage at a variety of frequencies. In this case the inverters can operate as a variable frequency drive to any motor saving equipment costs for VFD's and Transformers. The waterway with hydroelectric pump back and generation allows us to store energy during the day by pumping water uphill And generating power at night when solar power is non operational. Following highways and railroads allows this electrical power to operate vehicles and trains directly from the grid. Novel technologies allows cars to charge batteries while on the road be powered by the road and even cars that aren't electric can be moved down the road with the linear motor concept presented here.

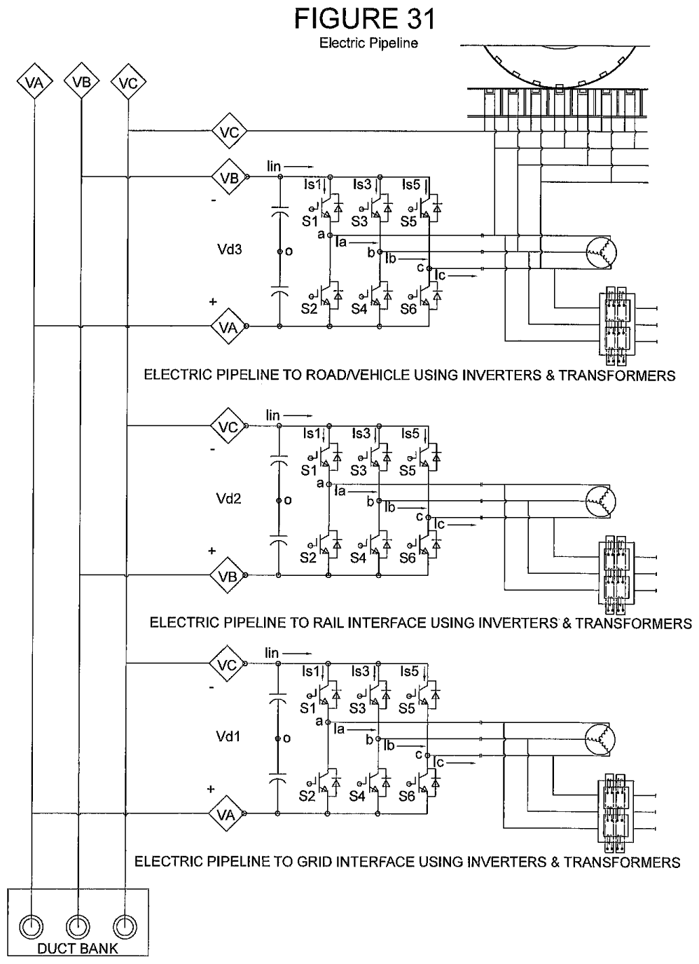

FIG. 31 shows DC to AC Inverters and Transformers that take the voltage differential between the two high voltage DC conductors, usually 15,200 VDC, and creates 3-phase AC power at the desired frequency. This can power pumps, compressors and other motor loads. The system can also create high voltage 3-phase AC power at 60 cycles frequency for the grid by powering a DC inverter with the DC voltage difference between the high voltage DC cables and the Neutral. The AC Current can then be adjusted by a transformer to the desired voltage. is a schematic depiction of a proposed method of transmission of electrical power depicted in FIG. 21 using two DC high voltage conductors with medium voltage differential between conductors plus a third conductor to act as a neutral. An electronic inverter will create medium AC voltage or pulse current anywhere along the corridor providing power for conventional motor or advanced linear motor driven equipment. 5G wireless networks will allow vehicles and locomotives to communicate with the electric pipeline drawing the exact power they need to move down the road or railroad.

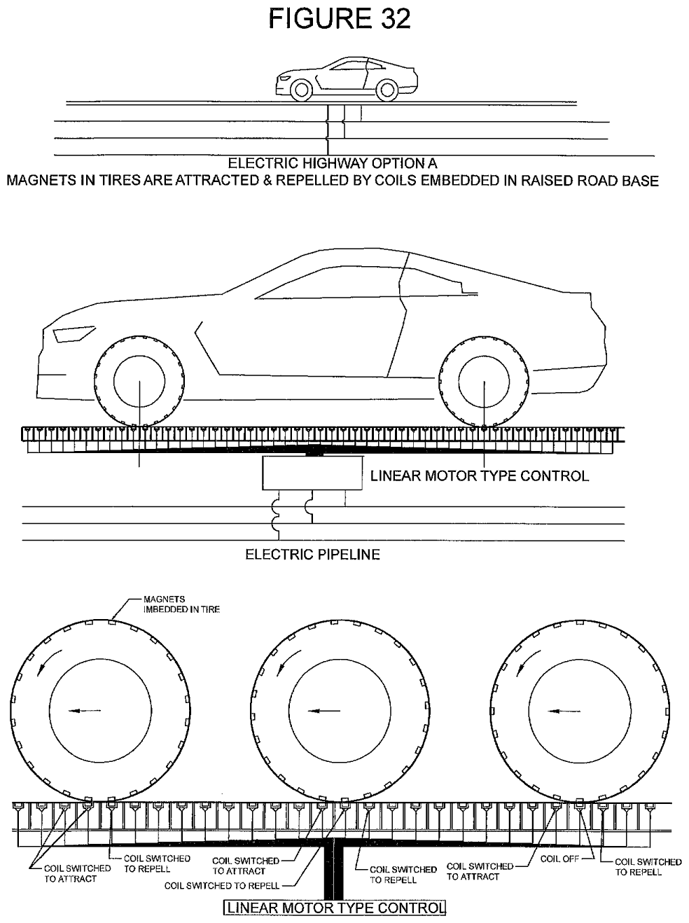

FIG. 32 is a schematic depiction of an expansion of the system depicted in FIG. 21 with the focus on a method for movement of electricity along highways while providing methods for moving vehicles along with the focus on the transfer of energy to non-electric powered vehicles through modified wheel to road interface. In this case magnets are installed just below the surface of the tires on a car or truck the spacing of these magnets Are a multiple of Pi. The coils embedded in the road base just under the surface are also separated by a distance of Pi so that the coil can be activated to attract the magnet at the exact time and then repel another magnet on the opposite side of the tire creating rotation in the tire leading to the movement of the vehicle down the road without internal power being applied the 5G wireless network will allow communication between the car and the electric road to time the injury and exit and the electric road and also as a method for billing for the electrical power used by the customer in the car. This would allow any car to be propelled down the highway saving billions of tons of CO2 from entering the atmosphere as a result of these cars burning gasoline or trucks burning diesel fuel it will also allow cars and trucks to move at high speeds because everything on the road is controlled by the 5G network. The cars and trucks can be separated safely and travel at much higher speeds and then when slowing can put power back into the grid. This would be a major contribution to the reduction of CO2 in the atmosphere.

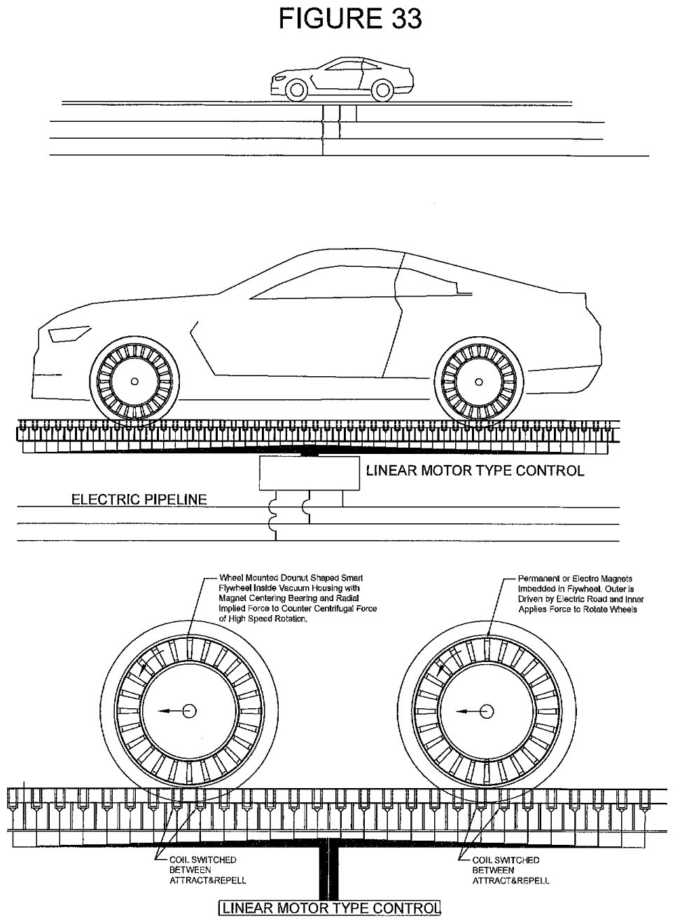

FIG. 33 is a schematic depiction of an expansion of the system depicted in FIG. 29 the transfer of electrical power to electric vehicles when stationary or moving through a flywheel arrangement located in the wheel area of the vehicle. This flywheel would be part of the cars hub or rim and would operate in a vacuum and very high speeds. The coils and the electric highway our positioned a little higher and the outside of the tire so that the flywheel can be accelerated by the coils. In this case the car does not have to be moving In order to store power. The flywheel is like a battery the faster it turns the more energy it store, so this method can be used at stoplights to keep the flywheels charged up while you're waiting in traffic this would keep the engine in the car from needing to operate most of the time. Once again millions of tons of CO2 would be kept out of the atmosphere because all the electricity power in these cars emits no CO2. This system could follow Interstate highways having a fast lane it operates like a toll road you pay a toll and you pay for your power but the savings and the time saved would make it extremely economical for both the vehicle operator and the power provider. The other component is how the flywheel imparts the power to drive the car since the flywheel is enclosed in a vacuum and is held center by magnetic bearings it can operate extremely high speeds it will also have magnets embedded in its outer surface and coils on the inside of this doughnut shape flywheel will activate and translate to flywheel energy to the wheel. Once again this would make any vehicle capable of being driven down the road by the electric highway simply by changing the rims on your vehicle. This would allow the millions of vehicles that aren't electric to use electricity when going down the road the highway the streets the busy streets in cities wherever the electric highway and pipeline go.

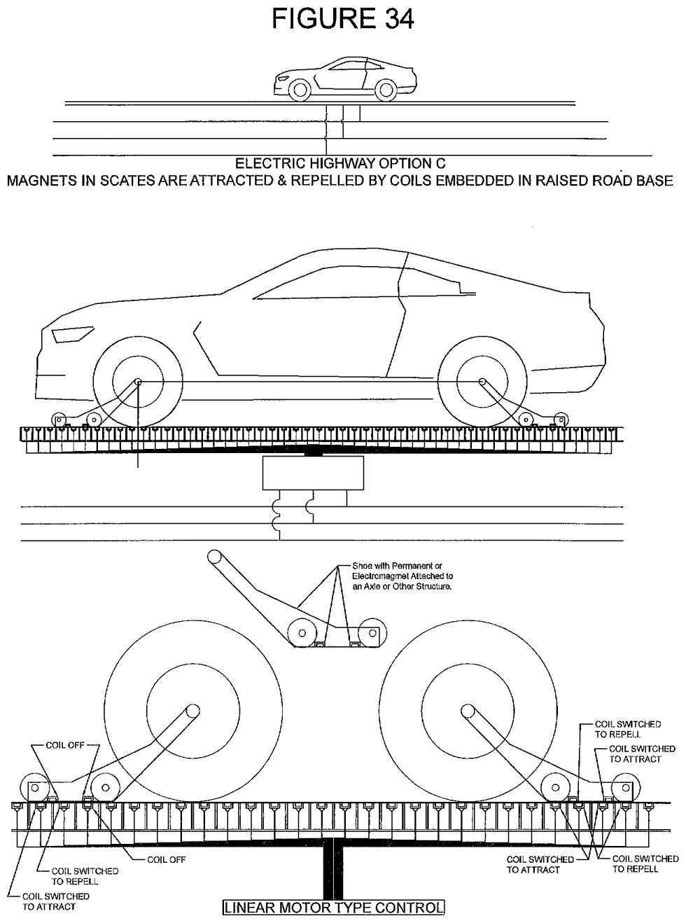

FIG. 34 is a schematic depiction of an expansion of the system depicted in FIG. 29 with the focus on a vehicle with an addition of skates that interface with the road through coil mechanisms. This is another method of taking ordinary vehicles and converting them to run on the electric highway. In this case skates would be attached to the bottom of the car typically around the front and rear axle on the left side. By the way only one track will have the electric highway coils embedded in it this allows quick exit from the electric highway, when your exit approaches it also makes it simpler and not having the match the wheelbase of all vehicles since only one side of the car or truck he's in the through or guide of the electric highway. The skates mentioned earlier deploy when the car is in position the magnets in this case Electro magnets in the skates will interface with the coils on the electric highway through the 5G wireless network these skates will push the vehicle down the road or slow it down. Once the destination exit is reached skates will retract and the car will continue on its own power to its destination. There is an opportunity to include a charging mechanism for electric cars into these gates whereas the car can be pushed down the road by this gate but also could generate power to charge batteries at the same time. This feature is made possible by the Electromagnets in both the electric highway and this gate allowing current to be produced along with mode of force.

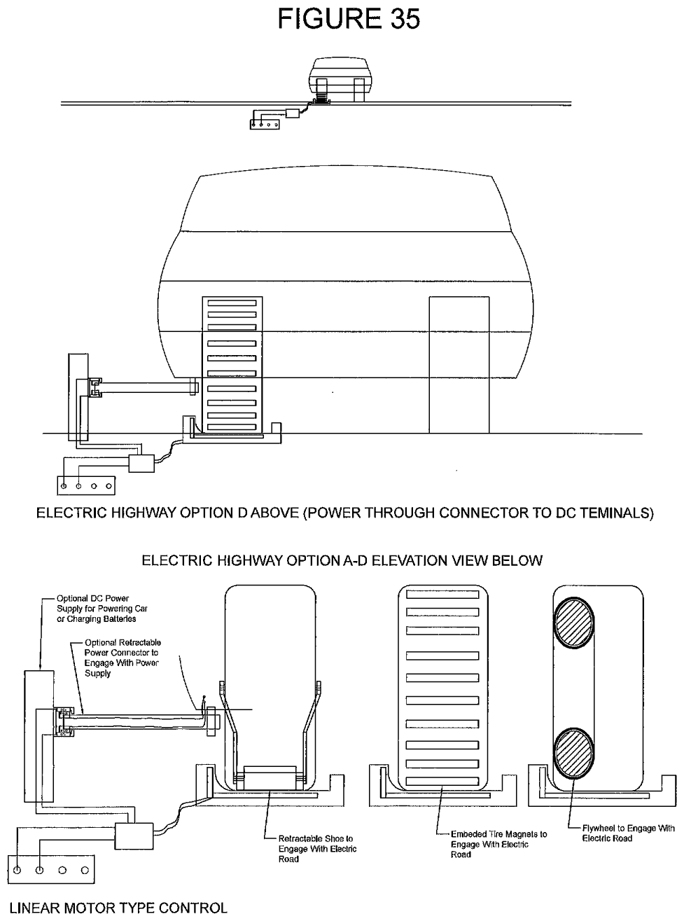

FIG. 35 shows additional details of the road to vehicle interface and a method of supplying DC power to an electric vehicle to charge or power the it as it moves down the road or is parked. Now we get to the electric cars that are already equipped to go down the road without burning fuel. The only problem is where do you find a place to charge your vehicle and what do you do and the hour or two that it takes to charge the vehicle you've lost time. In this figure we see the ability to plug in to a raised power source as a slot and rollers and contactors they can connect the electric car to DC voltage matching that required to charge your batteries. In this case they will drive the car with their own motors but instead of the battery running down it will either maintain or actually charge depending on the capacity of the circuit and the speed and terrain of the moving vehicle. There are many options on how to connect to the power source while stationary or moving, but the idea here is to get clean power into that electric vehicle battery so that they're not using coal-fired power plants to charge their batteries which actually can cause more greenhouse gas emission in the gasoline that other cars use. Rest areas along the highway can all have charging connections people that won't even have to get out of their car to use once again the 5G wireless network will build them for the power they consume And they continue down the road without worrying about where they're going to plug in next.

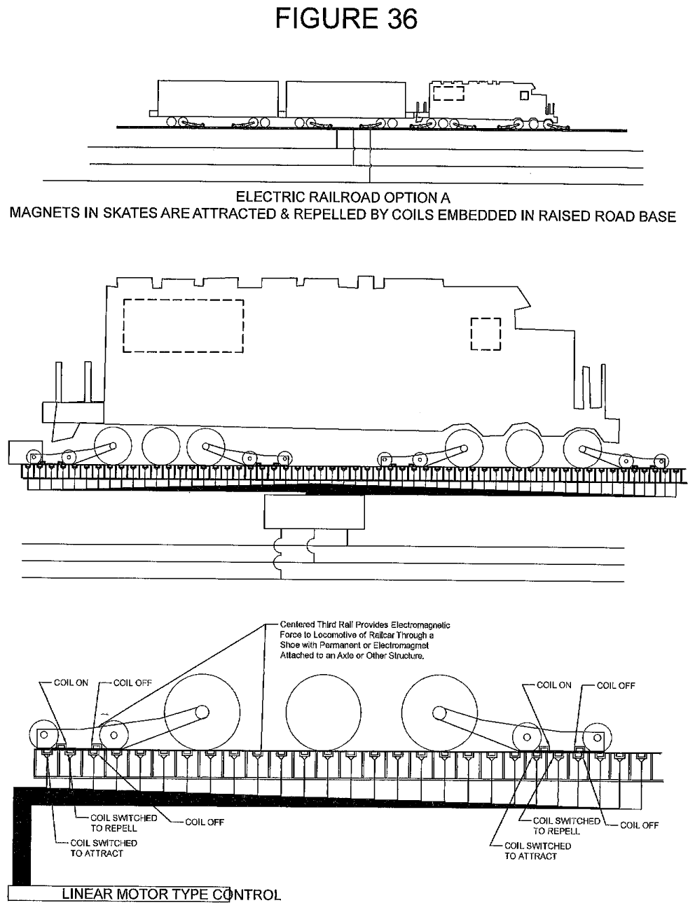

FIG. 36 is a schematic depiction of an expansion of the system depicted in FIG. 21 with the focus on a method for movement of electricity along railroads while providing methods for transfer of electrical power to electric powered railroad locomotives or railcars to provide power for the movement of the train. It also shows a schematic depiction of an expansion of the system depicted with the focus on the transfer of energy to railcars through modified shoe to third rail interface. The system also allows the ability to provide motive force direct to railcars providing power for the movement of each individual railcar. Electric trains are not new all across Europe and Asia electric trains are everywhere. There's a lot of equipment that has to be in that locomotive in order for the power they pick up to be used to drive the train. Transformers inverters variable frequency drives all have to be installed on every locomotive in order for them to take alternating current from the power lines usually running overhead. This technological approach attempts to drive the train from the third rail that can actually push it down the track. Even though there's a lot of weight on that train the fact that you could spread that out over many skate type drivers makes it possible to move the entire train even down to the individual colors using this abnormal approach of linear motor technology along with magnetic skates and third rail. Once again 5G network makes it possible for the rapid communication necessary to match the speed to the power to the grade to the wind resistance to the weight on the train all these things controlled by a central processor.

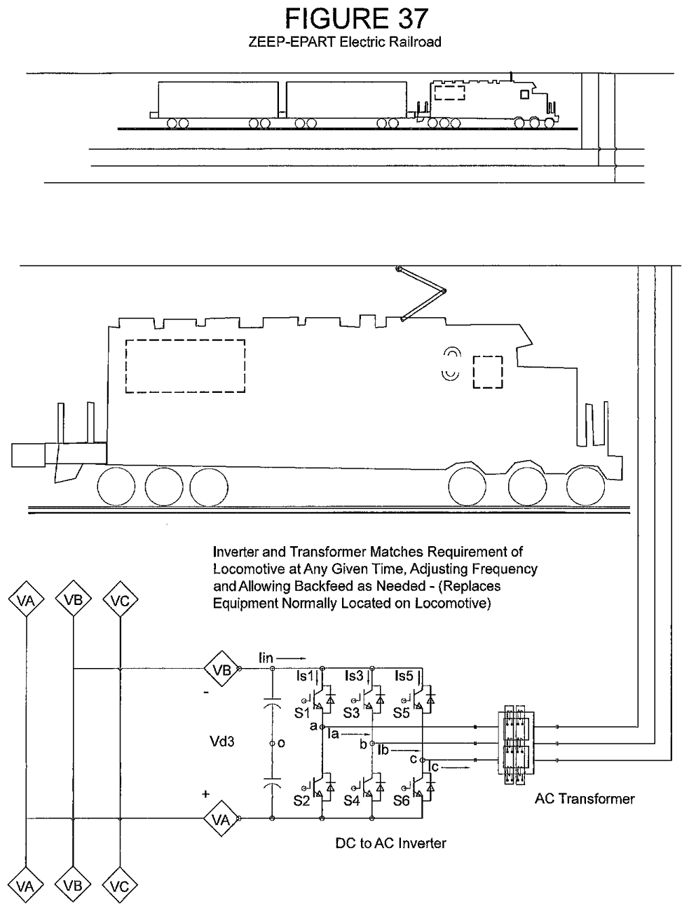

FIG. 37 is a schematic depiction of a system that provides electrical power to an electric locomotive that matches the precise voltage and frequency to match the speed, load, grade and resistance of the train through a 5G wireless connection. This eliminates the equipment on the locomotive like inverters, transformer and other control devices. Power can re-enter the grid when the train applies dynamic braking. Once again we have the opportunity to save money on equipment for each unit locomotive and sending the required voltage amperage and frequency to the locomotive motors on each wheel or axel. This along with back feeding the grid to slow down or when you go downhill makes the economics of the electric railroad superior to that of the highway in a lot of ways. The main barrier to electric trains are the power lines required to run it. But in the case of the electric pipeline we can run it along every railroad along every major highway and interconnected to the grid above the ground and make the entire grid system more stable and reliable and provide power to every moving vehicle traveling on major roads and railroads. This is all possible my using direct current high voltage but a novel method of creating medium voltage and low voltage linear motor control and also variable frequency alternating current at any voltage required.

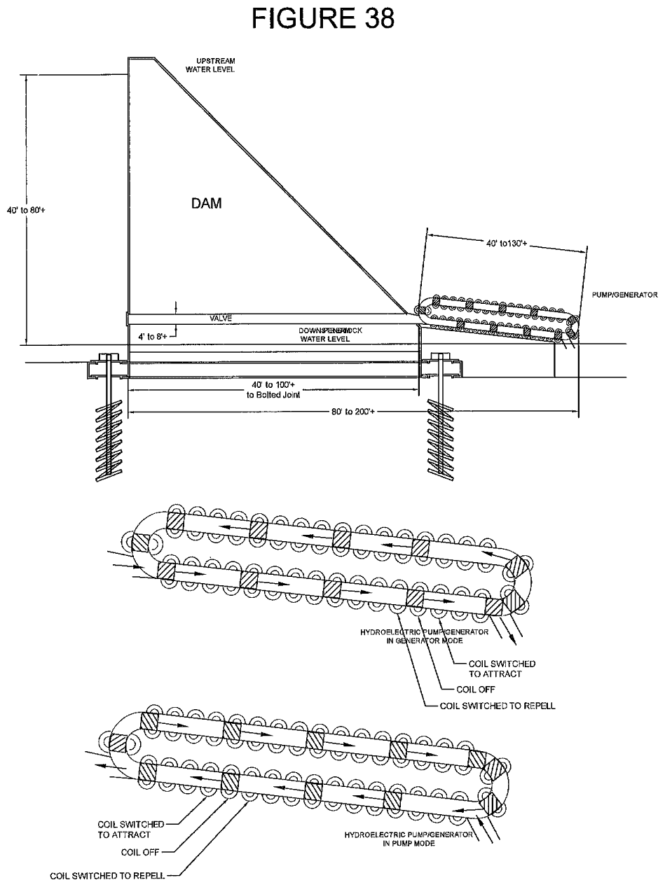

FIG. 38 is a schematic depiction of an expansion of the system depicted in FIG. 21 with the focus on a method for movement of electricity along waterways while providing methods for storage of electrical power for use during on-peak times using a hydroelectric pump back and generation system. The final type of transportation movement is that of the waterway. We could do the same thing that we're doing with vehicles and trains to the barges they travel the rivers all across The United States. This is more difficult because of the water it is curves Benz and bridges and other obstacles. While this can be done what this technology proposes to do is to transfer a lot of that freight to the high-speed electric trains. We do need a waterway traveling east to West to move water from where it's plentiful to where it's not plentiful. This gives us another opportunity to not only move water but move electricity from one end of the country to the other. It also gives us the opportunity to store power during the day by using solar energy to pump water uphill and then at night release the water downhill cheering power making solar a 24-hour power source. This can be done either by batteries or by chemicals but why not do it with water and get the water to the other side of the hill and in this case the other side of the continental divide where it can water the desert and offset irrigation water. This extra water can also grow the algae little captured the CO2 and sequester climate change by making the desert green with algae. A new type hydroelectric generator and pump is presented here that uses linear motor technology to move a piston through an endless pipe loop, capturing energy when water is released from a higher elevation to a lower elevation and we're storing energy by moving the piston and water from a lower elevation to a higher elevation. The only moving parts are the Pistons and the cost would be a fraction of the conventional hydroelectric generator pump back system. Another novel approach is the way the dam in channel are designed to minimize the height of the dam and the depth of the channel. This also limits environmental impacts because it follows rivers that reached the same elevations during flood stage. The dams can be fabricated at a central location and floated to the area needed Linfield with sand or cement to sink along with screw piles securing it in place. All these things keep the cost low enough to make the project economically feasible.

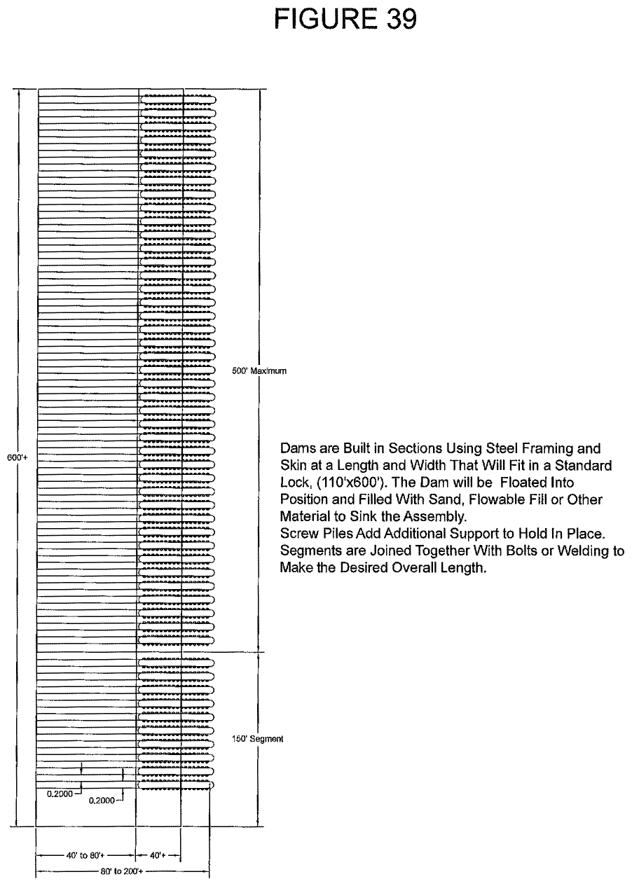

FIG. 39 is a schematic depiction of an expansion of the system depicted in FIG. 36 with the focus on the Dam. The dam can be fabricated out of steel and made to float the allowing it to be transported like a barge to the position it needs to be installed. The dam's maximum size to transport would be that which fits and the standard lock of the river system this is approximately 100 feet wide by 500 feet long. Dams that require more length would be made-up of sections up to 500 feet and bolted together to make the assembly is long as necessary. Bolted connections allow for ease of assembly on site prior to sinking using sand cement or other ballast.

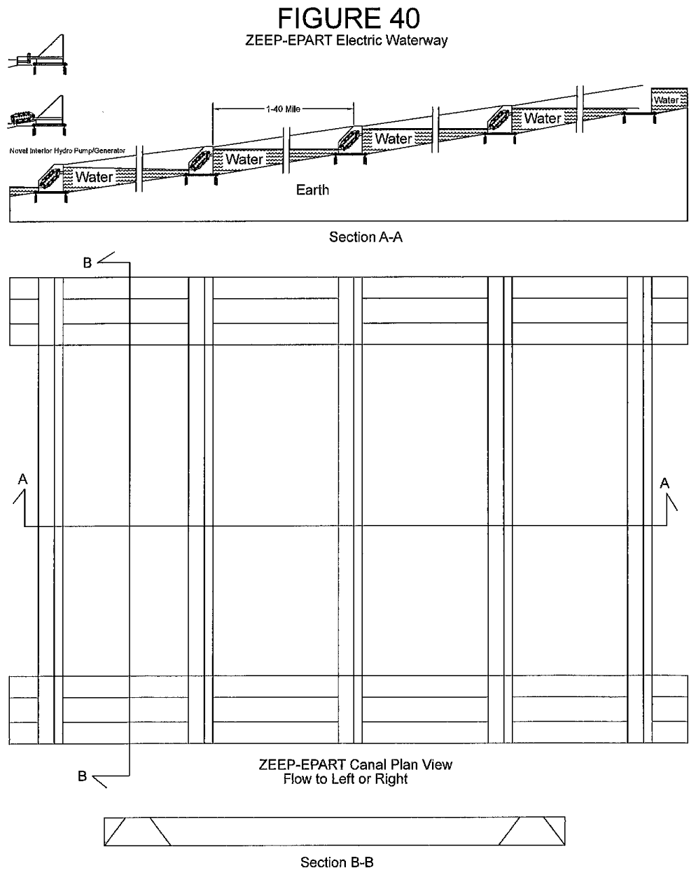

FIG. 40 is a schematic depiction of an expansion of the system depicted in FIG. 36 with the focus on the Channel. The channel is an earthen structure with a polymer lining to keep water from seeping out. The sides of the channel are sloped to maintain stability, ana begin below grade and end up to 80 feet above grade at the next dam. There are three possible pup/generator designs, Conventional and Interior and Exterior Linear Motor Piston type.

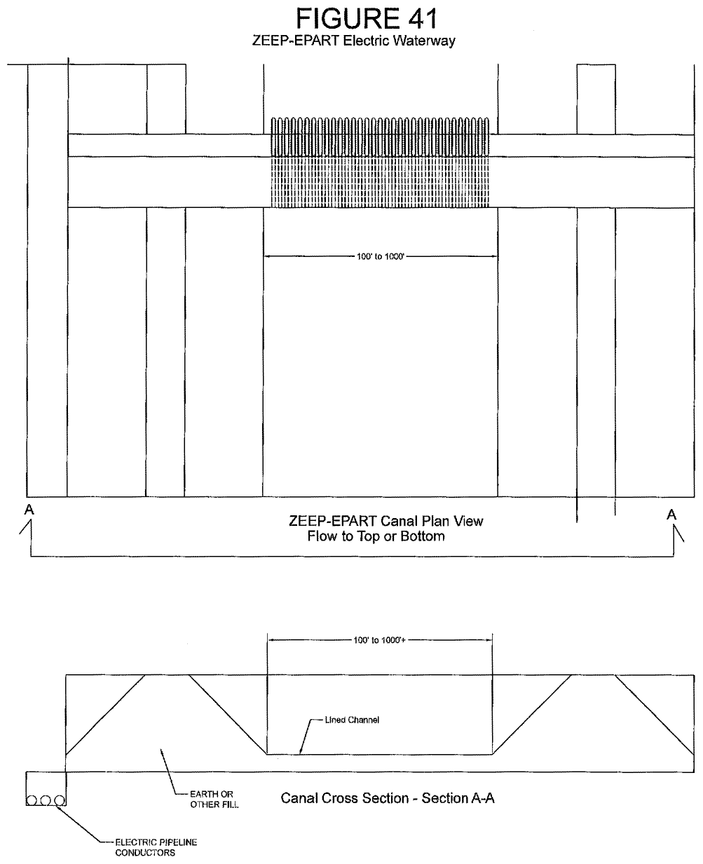

FIG. 41 is a schematic depiction of an expansion of the system depicted in FIG. 35 with the focus on the dam and channel design. This provides more detail on the channel and the dam layout and shows the cross section out of the channel including the liner.

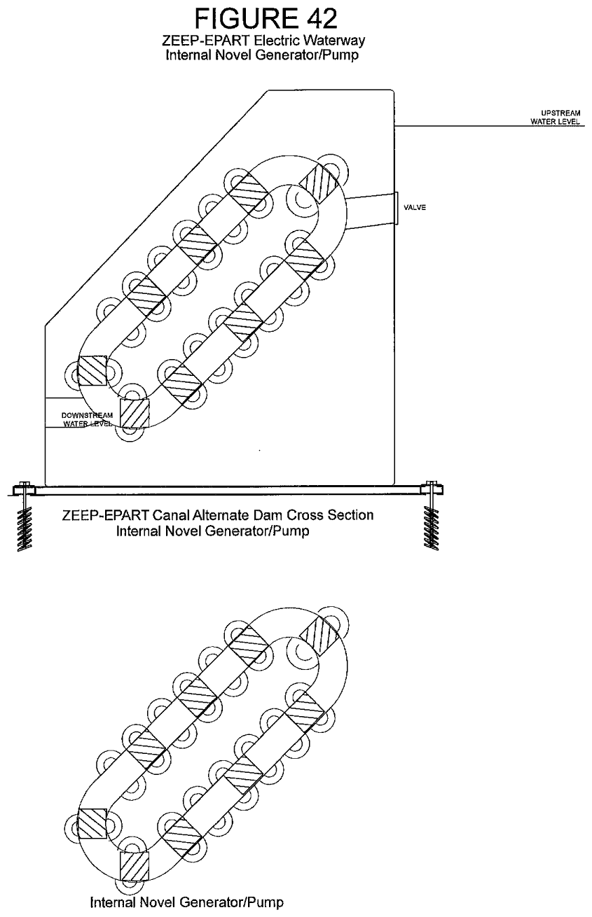

FIG. 42 is a schematic depiction of an expansion of the system depicted in FIG. 35 with the focus on an alternative dam interior. This design allows the pump generator to be installed inside the skin of the dam. Makes it easier to maintain and he's protected from the environment including UV rays and temperature variations. The operation of the piston and coils is the same as with the external model. High voltage DC pulses activate calls to either push or pull depends long the internal pipe. The bottom straight run of the pipe is where the power is produced or used to move the water the bands and top section are there just to get the piston back into position for our run or a power pump. The Pistons we'll stop to seal the pipe I'm getting water on the top side making it difficult to move the piston back into position. The Pistons are made of soft material but have a magnet embedded close to the surface so that the coils can have something to operate against. Provision will be made to access the Pistons for change out or rebuild as necessary. The coils themselves can provide strength to the pipe allowing the pipe to be as thin a wall as possible. The pressure on the pipe is based on the height of the dam and in most cases, we'll never exceed 80-PSI.

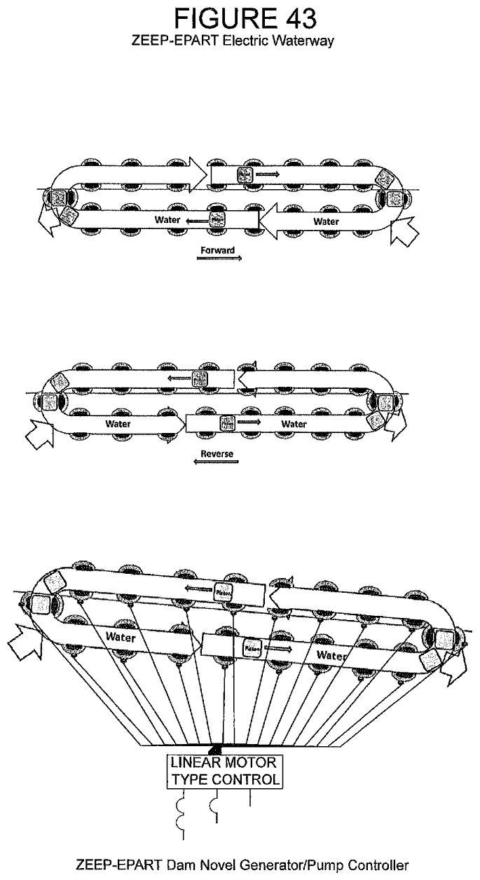

FIG. 43 is a schematic depiction of an expansion of the waterway system depicted in FIG. 35 with the focus on the transfer of electrical power to marine vessels providing power for the movement of the vessel along the waterway either through propeller, cable draw or linear water propulsion system. This figure also shows the possibility of using this design to power marine vessels, similar to a jet pump propelling a vessel through the water

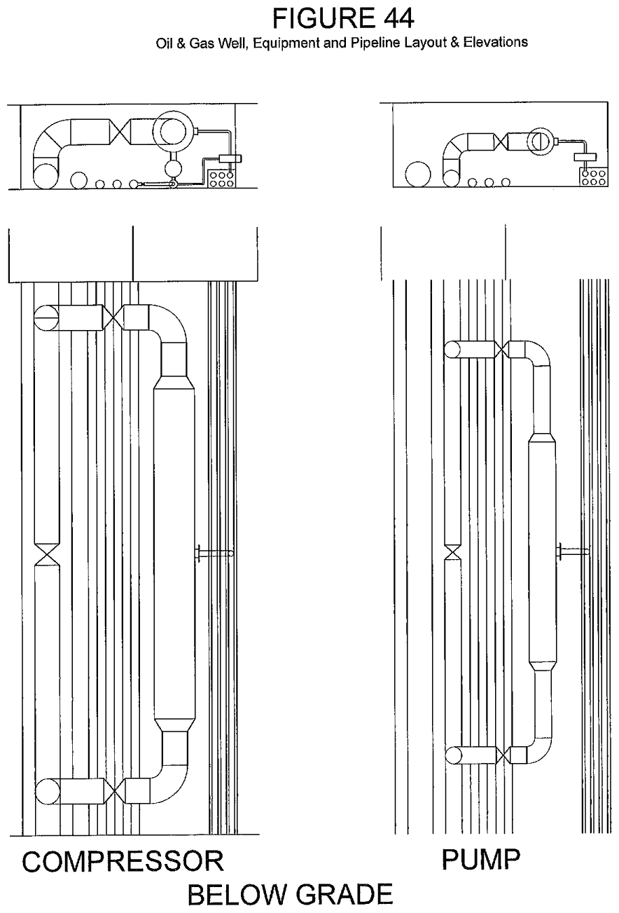

FIG. 44 is a schematic depiction of the embodiment depicted in FIG. 21 with the focus on pumping of oil, NGLs, water and CO2 and compression of natural gas. Electricity following the pipelines makes the position of pumps and compressors very versatile and can lower installation and operation costs. It also includes the interconnection with equipment such as pumps and compressors without penetrating the surface of the ground. Various pipelines delivering the products to market, combining the electrical transmission with the pipelines and the compressor and pump locations being optimized for terrain, pipe size and wall thickness, providing for a low environmental impact and lowest installation and operation cost. It also presents a variable speed function for motors driving pumps and compressors.

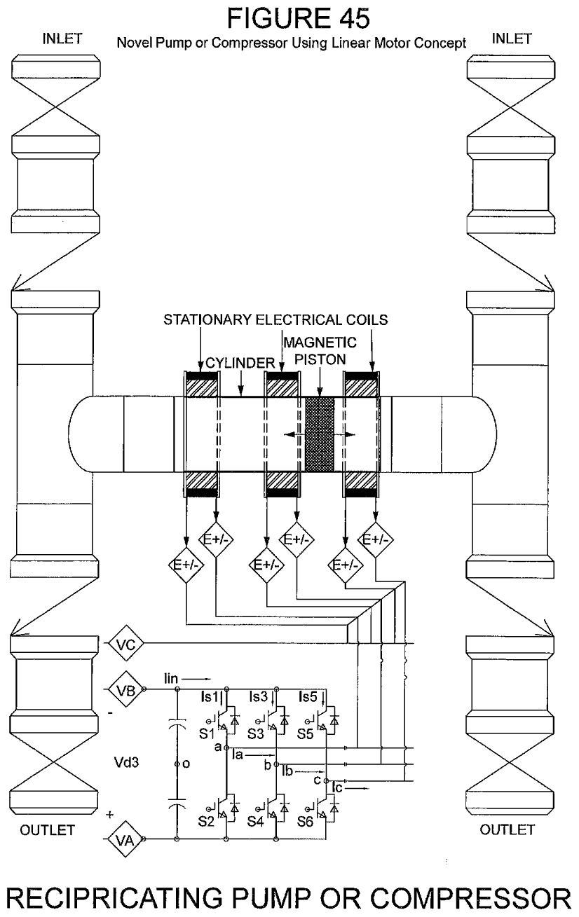

FIG. 45 is a schematic depiction of the embodiment depicted in FIG. 21 with a new concept pump and compressor concept allowing for components to stay underground and utilize electrical energy from the electric pipeline. The reciprocating pump or compressor uses linear motor technology and has only one moving part which is the piston. Since there are no seals to fail this novel pump or compressor will be free from emissions currently experienced in most pumps and compressors.

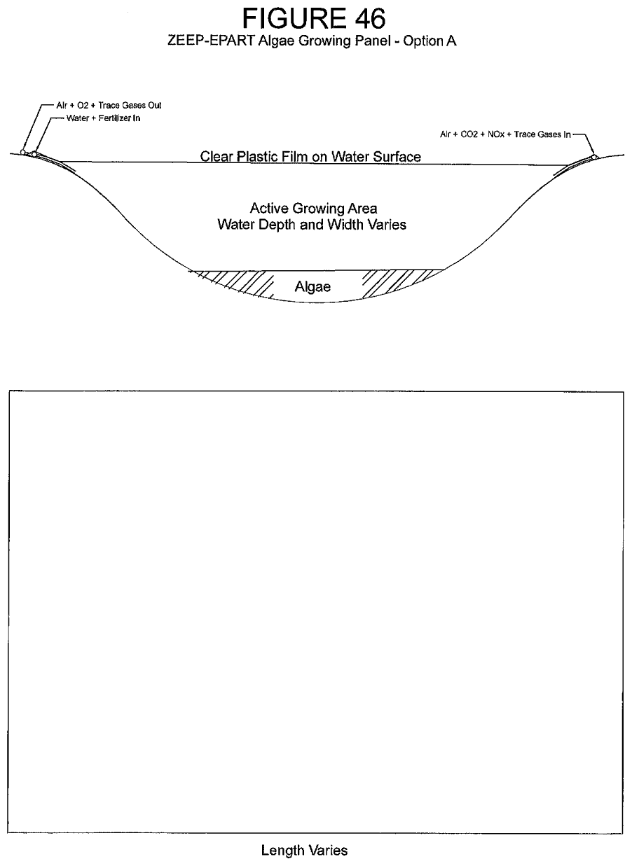

FIG. 46 depicts a system for growing algae in channels with thin film on water to reduce evaporation along with methods for supplying air, water, fertilizer, CO2 and algae remains in channel. The reason that the algae is staying in the channel is because part of the algae will be sequestered, keeping the CO2 that was captured by the algae out of the atmosphere. The algae will grow year after year until it reaches a depth that is the maximum for the channel width and depth. Then the algae will be completely enclosed with a Poly liner and covered with soil deep enough to keep it from decaying. Some methane will be produced just like it is in landfills, and this methane will be captured cleaned up and so or used for the process. The soil that covers the algae can we obtain from excavating a new channel adjacent to it.

FIG. 47 is a depiction of the algae of algae growing panels on slopped or flat roofs or on the ground. These algae panels are made-up of either flexible or rigid plastics. In one case we use an extruded plastic panel with round cross section approximately ½ inch thick. The round cross section is stronger than the square cross section but requires more plastic to build, therefore it will cost more. Another option is to use a standard double wall Mexican panel which has a square cross section but is readily available from plastic suppliers. Sense the algae will be very shallow with these panels, this would be a good place to use the semitransparent solar cell to shade the algae and gain electrical power from the solar cells. This type of panel could easily be affixed to a roof and the algae water mix can be delivered to it and returned from it using flexible tubing. The amount of weight added to the roof is insignificant, and it is a good use of space. The liquid circulating through the panel will shade the roof, reducing heat load inside the building. The next type algae panel is a flexible semicircular cross section similar to an inflatable Matt. This cross section is relatively strong and can lay on any flat surface including water and will allow an airspace for the delivery move CO2 and the removal of other gases such as oxygen. You've used on a roof caution must be taken not to have it full of water when there is the potential for additional rainfall to accumulate on the roof. In this case if rain occurred the panel can be drained and only a slight amount of weight would remain as part of the plastic. This type of flexible algae panel would work well on the ground provided that's some backing could be laid down to protect the bottom side of the panel. A body of water would also be a good place to use this type of panel considering that it will stay level and cool in the hot summers. The sizes very on this type panel from 4 inch to 8 inch depending on the growth rate potential along with intensity limitations of the algae.

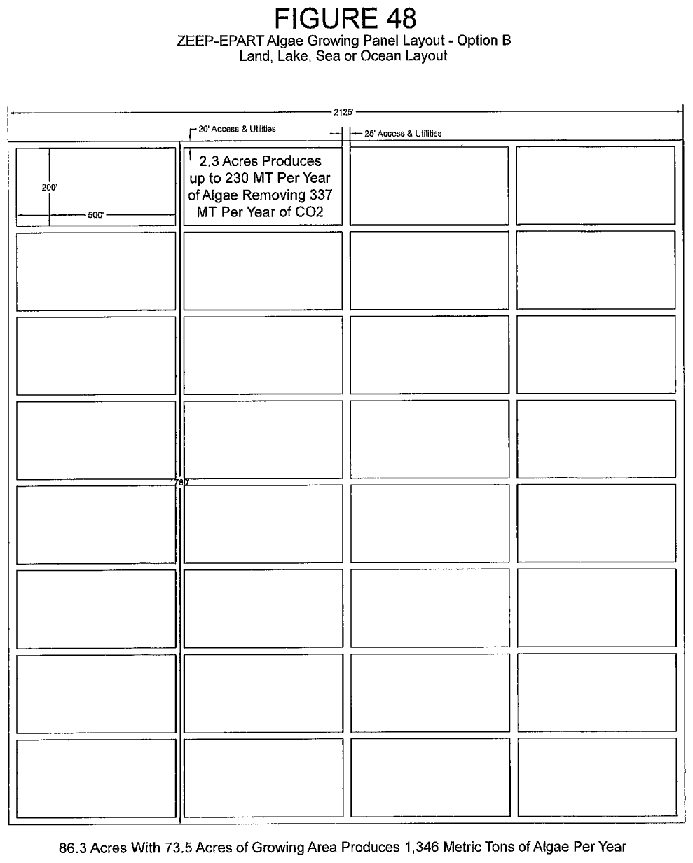

FIG. 48 is a depiction of the algae growing panels on land. This figure describes the layout for an 86-acre plot of land to maximize growing capability while still allowing access around the panel. It is projected that this size algae growing area would produce 1346 metric tons of algae per year. Each plot which is 200 feet by 500 feet would produce 230 metric tons of allergy per year removing 337 metric ton per year of CO2 from the atmosphere or from a generation source.

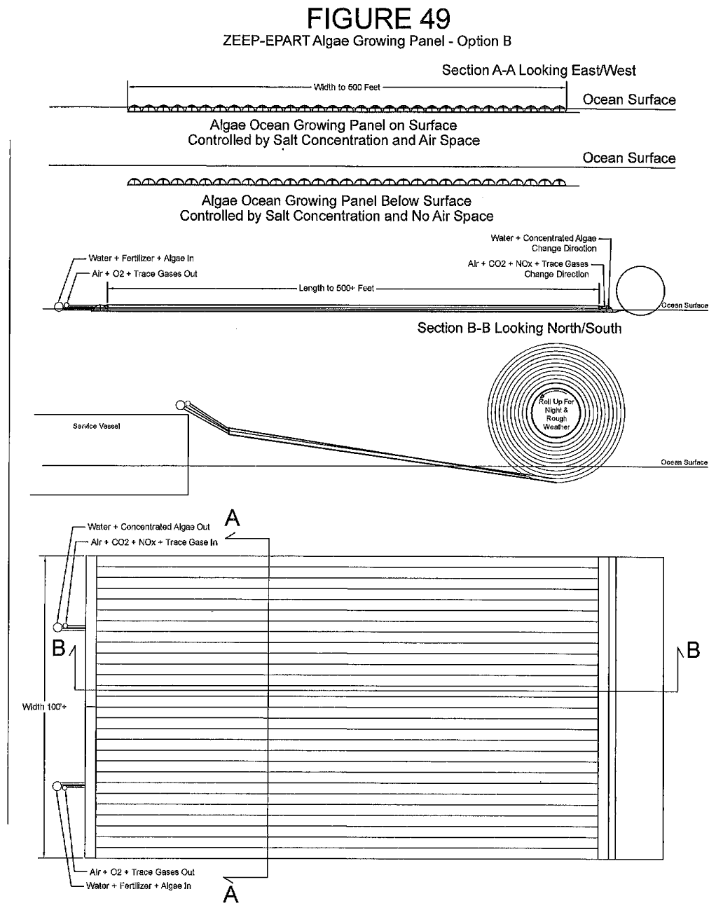

FIG. 49 is a depiction of the algae growing panels on lakes, seas or oceans. This installation has options 4 deploying the panels during the day and retracting them overnight or during bad weather. This type arrangement would work well in an ocean or sea or in a large lake. The retractable section uses a drum that rotates with the aid of paddles submerged in the water and it rolls the algae panel toward the service vessel. This will allow the panel to be drained of algae and stored overnight and can easily be deployed by inflating the algae panel and it will unroll itself.

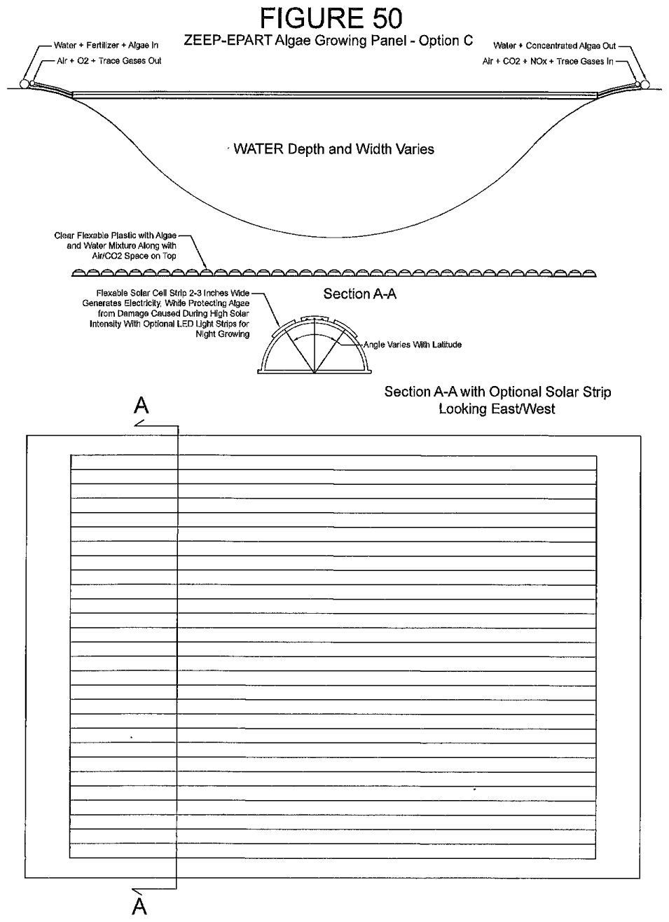

FIG. 50 is a depiction of the algae panel on channels with solar cells to generate electrical power while shading algae from intense sunlight. Options for LED lights for nighttime growing. Flexible solar cell strip 2-3 inches wide generates electricity, while protecting algae from damage caused during high solar intensity with optional led light strips for night growing. Clear flexible plastic with algae and water mixture along with air/CO2 space on top

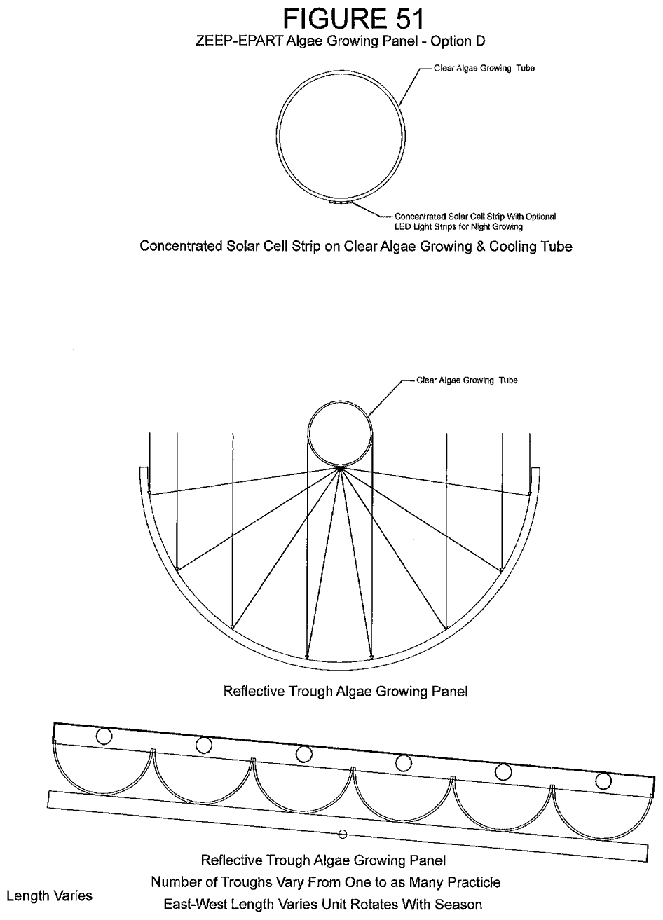

FIG. 51 is a depiction of an algae panel that uses a trough to focus sunlight along a thin concentrated solar strip on the bottom of a clear glass or poly tube. Clear tube allows algae to grow using direct and scattered sunlight while cooling the concentrated solar cells. This arrangement has an advantage of focusing the light have high efficiency solar cells and gives the best of both worlds allowing algae to have sufficient sunlight to grow in having solar cells that are smaller but produce as much energy as a non-concentrated solar cell. The LED lights on the backside of the solar cells in a low for 24 hours per day growing of the algae which would easily make up for the loss of the solar intensity during the day. This panel would be arranged so that the long runs of through would be in an east West direction and the panel could be tilted to maximum solar intensity during the day as the sun moves higher in the sky during midday and lower in the sky during morning and evening. This panel also has the ability to cool itself with the circulating water analogy preventing the solar cells from overheating. The overall cost of this panel would be lower than equivalent power production and algae production equivalence.

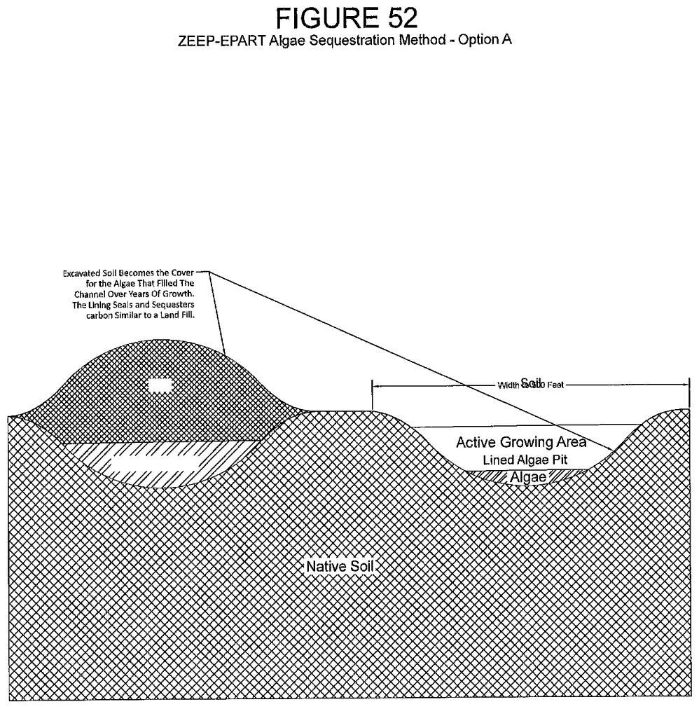

FIG. 52 is a depiction of an algae sequestration method for algae growing system given in FIG. 46. This sequestration method was discussed earlier and uses the continuous growing algae in channel method then covered and impounded underlining using soil from an adjacent excavation this allows the sequestration of the algae and low cost and it's close enough to the surface to allow methane to be collected for use by the process or cleaned up and sold as a commodity. Automated excavation methods can be used to lower costs on this type of algae production and sequestration.

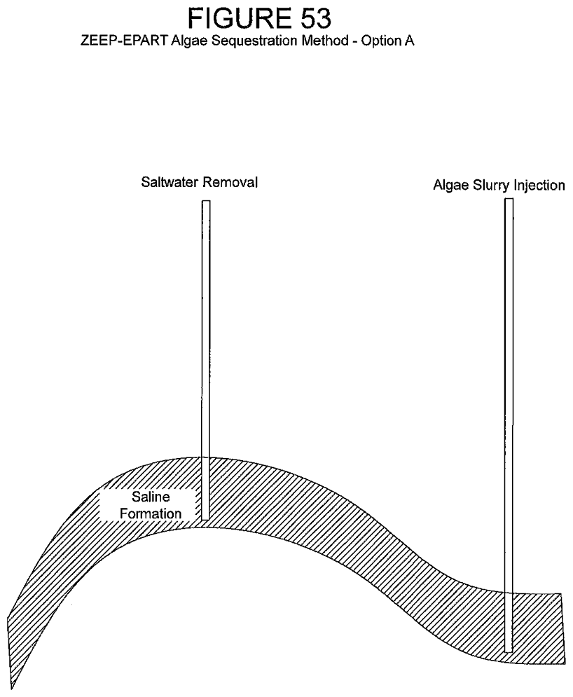

FIG. 53 is a depiction of the algae sequestration in deep saline formations or depleted oil-bearing formation with algae being slowly converted to methane and other hydrocarbons resulting in renewable oil and natural gas. This sequestration method stores algae deep underground where it can be locked away for an extended period of time even hundreds of years however there's still the opportunity to bring hydrocarbons back to the surface at a future time to produce electrical power and to continue this process indefinitely. The correct formation will be identified to maximize the algae that can fit into the formation for space this technology also includes methods for removal treatment and conversion into freshwater the water that comes from the formation which makes more room for algae to be sequestered underground. Along with the freshwater sodium chloride, calcium chloride and by products any lithium in the formation would pay for the entire process just in the recovery of the lithium or iodine.

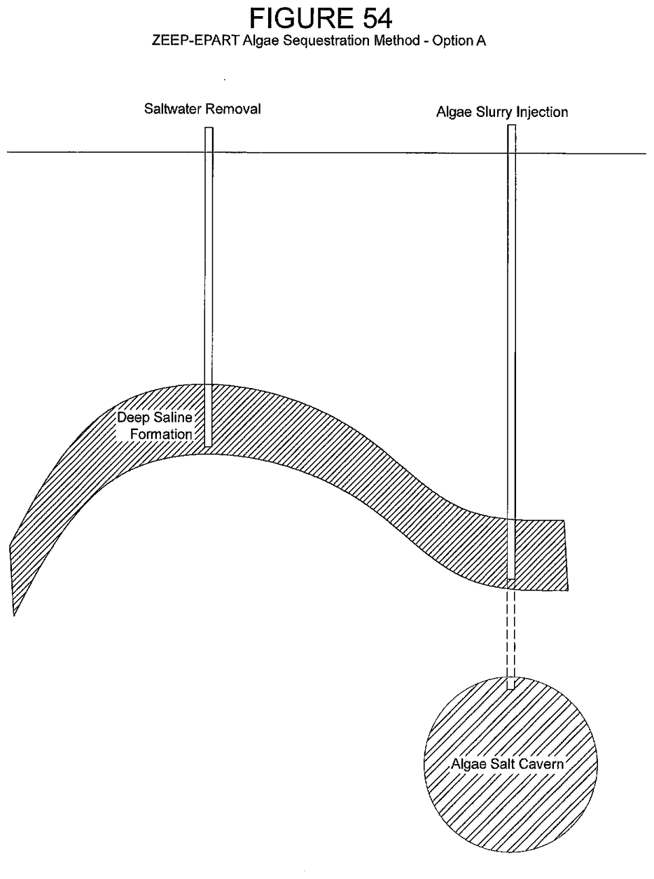

FIG. 54 is a depiction of the algae sequestration in depleted oil and gas formations, Saline Formation or Salt Cavern. Salt caverns are a unique and cost-effective manner for sequestering large quantities of algae. These deep formations are created as water is injected it is solved formations dissolving the salt creating voids. This method allows the algae to be held in place with no biological degradation due to the high concentration of salt in the formation. Depending on the size of the salt formation, millions of tons of algae could be stored there for future use as a renewable fuel. Methane will always be produced to some degree that can also be collected and run to the process with it saving costs and keeping the pressure low in the formation.

What is claimed is:

1. A system for producing electrical energy and coproducts, the system comprising:
a Zero Emissions Engine (ZEE) comprising (i) a turbine engine or other combustion engine and (ii) a first electrical generator, the turbine engine or other combustion engine combusting a gas fuel with air or other oxygen source;
a boiler or other heat exchanger, for heat recovery, which produces steam from heat recovered from an exhaust of the ZEE;
a steam turbine which is driven by the steam produced by the boiler or other heat exchanger;
a second electrical generator which is driven by the steam turbine;
a saltwater treatment system in which steam exhausted from the steam turbine and a waste heat exhaust stream from the boiler or other heat exchanger is used to flash dry a saltwater concentrate and then preheat saltwater by direct contact such that CO₂ from the waste heat exhaust stream is absorbed by the saltwater.

2. The system of claim 1 further comprising an amine system which recovers additional CO₂ from the waste heat exhaust stream which is routed to a pipeline for sequestration for enhanced oil recovery or deep saline formation.

3. The system of claim 1 being supplied fuel from alternate methods including:
gasification, anaerobic digestion, or torrefaction of biomass, consisting of algae, food waste, agriculture waste, tree waste, or other source.
conversion of natural gas to hydrogen by pyrolysis or partial oxidation/steam reformer with water shift and CO2 separation.

4. The system of claim 1 further comprising the turbine engine or other combustion engine being a piston engine with the first electrical generator being a built-in linear generator of the piston engine.

5. The system of claim 1 being co-located with a natural gas plant to allow for the use of an air separation plant that supplies liquid oxygen for combustion of methane resulting in exhaust made up of water and CO2 allowing ease of CO2 separation and sequestration by EOR, along with the liquid nitrogen being used as a refrigerant for the wet natural gas processing, and the production additional coproducts including ammonia by electrolysis or harbor-bosch process, polyethylene and PVC.

6. The system of claim 5 with methods for the improved of oil and gas production methods that include:
well layout with CO2 injection well in center of production that will initially produce oil and gas allowing for the location of the gas plant and zero emissions electrical plant that will supply power for drilling operations on surrounding wells.
a new method of drilling additional holes along vertical and horizontal wells using a tube rig supported micro drill that could replace fracking in areas that do not allow it.
allow for maximum production of oil and gas from a formation by using EOR from the beginning of operation, pushing a mixture of CO2, water and sand from the center of the formation to an ever-increasing radius as new wells are drilled.
resulting in zero emissions oil and gas production to support the point of use pyrolysis Production of hydrogen from natural gas, resulting in no carbon emissions along with the carbon feedstock support for a novel hydrogen storage device using activated carbon.

7. A method of producing a calcium conductor for underground use comprising steps of:
heating calcium chloride and one or more salts to a molten state in an electrochemical cell having an anode and a cathode;
applying a DC voltage across the anode and the cathode to induce an electrical current which passes through the electrochemical cell to produce chlorine at the anode and a calcium metal at the cathode, the calcium metal forming one or more rods; and
applying an aluminum surface treatment to each of the one or more rods while purging with an inert gas.
enclosing each of the rods in a protective sheath.

8. The method of claim 7 further comprising:
the one or more rods being a plurality of the rods and
the method further comprises
joining the rods together sequentially to form an electrical power pipeline and
burying the electrical power pipeline underground.

9. The method of claim 8 further comprising joining each adjacent pair of the rods in the sequence together by:
tapping an end of a first one of the adjacent pair of the rods wherein the tapped end receives a female portion of a threaded joining assembly formed of aluminum or other conductive material;
attaching a male portion of the threaded joining assembly to an opposing end of a second one of the adjacent pair of the rods; and
surrounding the threaded joining assembly with a sheathing.

10. The method of claim 9 further comprising of burying three of these conductors underground and using them to transmit electrical power long distances by:
by applying DC voltage to two of these conductors of up to 1-million volts, with a small difference in voltage between these two conductors of up to 50,000 volts.
the third conductor being a neutral.
then using an inverter across these two conductors and neutral to produce 3-phase AC current, adjusting the voltage through transformers then connecting the electrical power to the grid or novel electric road, electric railroad, and electric waterway.

11. The method of claim 10 further comprising an electric road where the conductors follow roads and highways long distances and interfaces with the vehicles through a novel method of linear motor related mechanisms including:
electric coils imbedded in a raised polymer track that are controlled by an inverter to match the required polarity of magnets imbedded in the tires of vehicles that propel the vehicle at high speeds using the computer controlled electric road interfacing with the vehicle with a 5G network for destination and billing allowing driver disengagement.
electric coils imbedded in a raised polymer track that are controlled by an inverter to match the required polarity of electromagnets imbedded in a flywheel mounted on the hub of the vehicles that speed up the flywheel that in turn supplies electrical power to an electric vehicle or applies torque to the vehicle wheel of a non-electric vehicle.
direct electrical connection to an electric vehicle using an induction interface, mechanical interface or skate interface with electric coils imbedded in a raised polymer track.

12. The method of claim 10 further comprising an electric railroad where the conductors follow railroads long distances and interfaces with the locomotives through a novel method of linear motor related mechanisms including:
electric coils imbedded along the track are controlled by an inverter to match the required polarity of electromagnets imbedded in skates that are installed at various locations on locomotives or even individual cars. this provides electromotive force to start and stop or sustain movement of the train. the computer controlled electric railroad interfaces with the locomotive and engineer with a 5G network for destination, sidetrack coordination and billing, allowing for safer operation.
direct electrical connection to a locomotive variable frequency AC power to match required speed and grade considerations.
direct electrical connection to a locomotive using conventional interface.

13. The method of claim 10 further comprising an electric waterway where existing rivers can be used to move water long distances, up considerable grades, allowing for the temporary storage of electricity in the form of potential energy, all while moving water from areas of plenty to areas of scarcity, using:
novel dam construction procedures consisting of portable metal structures bolted or welded together forming dam with elevations of 40 to 80 feet accommodating a conventional or novel and hydroelectric pump/generator, continuously pumping water uphill during daytime hours using internally produces zero emissions electricity or solar, wind, other renewable or conventional source. during nighttime, pumping stops and water returns downhill or down the other side of the hill.
novel hydroelectric pump/generator that consists of a large diameter tube constructed in an endless loop lined with electromagnetic coils that interact with several moving pistons with imbedded magnets controlled through a linear motor related mechanisms including a computer controlled inverter to match the required polarity of magnets imbedded in the pistons that pump the water or generate power by reversing the direction of the pistons.

14. The method of claim 10 further comprising an underground electric and coproduct corridor to and from the oil and gas production region allowing all upstream and downstream electric power and other coproduct requirements to be supplied with zero emissions power and coproducts. this supports the carbon reduction of the oil and gas industry and provides carbon free natural gas for the novel point of use hydrogen from natural gas concept by providing:
zero emissions power for drilling and completing activities along with the HDPE pipeline for water supply for fracking or other use, then converting it to a gas pipeline to eliminate flaring all together.
novel underground pump or compressor that uses seal less linear motor-controlled piston and cylinders to efficiently move products into and out of production area with little or no environmental impact.
power conventional pumps and compressors using the electric pipeline for built-in variable frequency-controlled power supply.

15. A method to transition the oil and gas industry into a renewable energy industry by incorporating algae production and sequestration into the operations of the production region by providing methods and all support products and feedstocks into the centralized gas processing and power production site and the system comprising:
growing gigatons of algae that captures CO2 from engine exhaust or directly from the atmosphere using the algae production panel, APP,
by processing and sequestering the algae for hundreds of years in depleted oil and gas formations, other underground saltwater formations, caverns or voids and surface impoundments created by excavation and covered by membrane and soil or water using the algae related technologies, ART;
providing a future source of renewable oil and gas (methane) utilizing the same infrastructure that currently distributes oil and gas.

16. Methods to produce algae from claim 15 using sunlight contained in clear polyethylene or similar flexible or rigid material formed in ways given in figures with the circular cross section filled half with liquid and half with air and CO2 with:
options for mounting on roof or inclined surface or on a flat surface or body of water.
water and lean algae mixture enter one end (a) and nutrients, combustion exhaust and/or air containing CO2 enter the other end (b) while oxygen rich air exits one end (a) and water, concentrated algae mixture and remaining nutrients exit the other end (b).
oxygen rich air can be directed to a combustion device or discharged to the atmosphere.

17. The concentrated algae mixture from claim 16 being dewatered and directed to underground formation for up to 100-year sequestration and subsequent breakdown into renewable oil and natural gas by way of pressure/thermal.
alternate for other sequestration of algae slurry in covered pits, underground caverns or at bottom of sea or lake.
methods for the diversion of some of the algae to produce food grade protein, animal and fish feeds, renewable transportation fuels, hydrogen, and methane.

18. Methods to produce algae given in claim 16 with:
the top surface of the circular cross section is covered with semitransparent flexible solar cells for the generation of electrical power with enough solar penetration to drive algae production.
bottom surface affixed with LED lights tuned to produce algae allowing algae growth during non-daylight hours.
methods for storing electricity using water pump back technology for use during nighttime hours.

19. Methods to produce algae given in claim 16 using non solar methods of producing algae including concentrated LED lights with:
closed system reactor optimized for algae production around the clock.
nutrient and CO2 addition optimized for maximum algae production for a given volume of vessel or body of water.
methods for maximizing contact between algae slurry, CO2, and nutrients through circulation devices.
concentrated algae mixture is dewatered and directed to underground formation for up to 100-year sequestration and subsequent breakdown into renewable oil and natural gas.
alternate for other sequestration of algae slurry in covered pits, underground caverns or at bottom of sea or lake.
methods for the diversion of some of the algae to produce food grade protein, animal and fish feeds, renewable transportation fuels, hydrogen, and methane.

20. Methods to produce algae given in claim 16 using solar energy concentrated with a trough lined with reflective material focused on the bottom portion of a clear or opaque pipe covered with high efficiency solar cells with:
cooling water circulating in the pipe keeps the solar cells cool to maintain efficiency.
with algae along with nutrients and CO2 included in clear pipes utilizing the direct and reflected solar energy to optimize and maximize algae production while cooling the solar cells.
including methods for tilting and rotating the trough to maximize solar intensity.
concentrated algae mixture is dewatered and directed to underground formation for up to 100-year sequestration and subsequent breakdown into renewable oil and natural gas.
alternate for other sequestration of algae slurry in covered pits, underground caverns or at bottom of sea or lake.
methods for the diversion of some of the algae to produce food grade protein, animal and fish feeds, renewable transportation fuels, hydrogen, and methane.
methods for the utilization of ammonia as a hydrogen carrier without the loss of the nitrogen benefit as a fertilizer.
by injecting ammonia into a water solution and passing it through a catalyst that releases hydrogen and combines the nitrogen with oxygen to form nitrates to support plant or algae growth.
allowing the hydrogen to be oxidized for electrical power production, heating or process needs.
